(19) United States
(12) Patent Application Publication
(10) Pub. No.: US 2025/0162973 A1
(43) Pub. Date: May 22, 2025

(54) METHODS OF SYNTHESIS AND USES OF AGRIMOL COMPOUNDS

(71) Applicants: Versitech Limited, Hong Kong (CN); Laboratory for Synthetic Chemistry and Chemical Biology Limited, Hong Kong (CN)

(72) Inventors: Chi Ming CHE, Hong Kong (CN); Zhiwen FU, Hong Kong (CN); Yiu Chung Gulice LEUNG, Hong Kong (CN)

(21) Appl. No.: 18/840,385

(22) PCT Filed: Feb. 21, 2023
(86) PCT No.: PCT/CN2023/077416

§ 371 Date: Aug. 21, 2024

Related U.S. Application Data
(60) Provisional application No. 63/312,755, filed on Feb. 22, 2022.

(51) Int. Cl. C07C 67/04; A61K 31/12; A61K 31/192; A61P 35/00; C07C 45/69

(52) U.S. Cl. CPC C07C 67/04; A61K 31/12; A61K 31/192; A61P 35/00; C07C 45/69

(57) ABSTRACT

Synthetic agrimol B and derivatives thereof (“compounds”) that have anticancer properties are disclosed. Methods for synthesizing the compounds with an overall yield ≥7% are disclosed. Pharmaceutical formulations in forms suitable for the delivery of the compounds to a subject in need thereof are disclosed. Methods of using the compounds for treating a cancer, reducing a cancer, or treating or ameliorating one or more symptoms associated with a cancer, such as non-small-cell lung cancer, in a subject are also disclosed. The methods include (i) administering to the subject the pharmaceutical formulation containing one or more compounds, for one or more times. The compounds can selectively kill cancer cells and/or cancer stem cells over noncancerous cells by inducing mitochondria dysfunction and/or inhibiting ER to Golgi trafficking in the cancer cells and/or cancer stem cells.

### FIELD OF THE INVENTION

This invention is generally in the field of agrimol B and its derivatives and methods of synthesis and uses thereof.

### BACKGROUND OF THE INVENTION

Cancer, also known as malignant tumor, is a common and frequently occurring disease that threatens human health. Lung cancer is the main cause of death of cancer patients. There are many treatments for lung cancer, among which surgery is the first choice. However, a vast majority of lung cancer patients are diagnosed as advanced stage at the first visit and have a high recurrence rate after surgery. Postoperative adjuvant chemotherapy is an effective strategy to prevent recurrence and metastasis. With the recent development of molecular biology, more and more targeted proteins have been excavated, which provide new developed drugs according to the identified targets for the treatment of advanced lung cancer.

Bioactive components isolated from plants are important sources for anticancer drug discovery and development in this regard. Over the last decades, research on natural products provides a large number of potential lead compounds with unique scaffolds and mechanisms of action. Agrimol B (AGB) is a trimetric acylphloroglucinol compound and was originally isolated from Agrimonia pilosa. Although AGB was obtained from the plants for several decades, little was previously studied about the activity and application of AGB. Additionally, isolation of AGB from plants is a laborious and time-consuming work and the amount of AGB in the plant is extremely low, which limit its further development and application. Previous reports of AGB synthesis suffer from low yield, complicated synthesis, and low atom efficiency (Li, et al., Acta Chimica Sinica, 36(1):43-48 (1978); Pei, et al., Acta Pharmaceutica Sinica, (1989) 431-437; and CN108264454 by Huang, et al.).

There remains a need to develop compounds that process anticancer properties and synthesis of these compounds having an improved overall yield.

Therefore, it is the object of the present invention to provide AGB derivatives that possess anticancer properties.

It is a further object of the present invention to provide methods of synthesizing AGB and AGB derivatives with an improved overall yield.

It is a further object of the present invention to provide methods of using AGB and AGB derivatives as anticancer agents.

### SUMMARY OF THE INVENTION

AGB and AGB derivatives (also referred to herein as “compounds”) that possess anticancer properties have been developed. The AGB or derivatives of AGB can have the structure of Formula I:

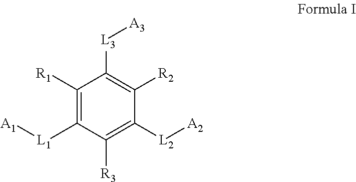

wherein: (a) L₁ and L₂ can be independently a bond (single, double, or triple), a substituted or unsubstituted alkyl, a substituted or unsubstituted alkenyl, a carbonyl, or an alkoxy; (b) L₃ can be a bond (single, double, or triple), a substituted or unsubstituted alkyl, or a carbonyl; (c) A₁ and A₂ can be independently a hydrogen, a substituted or unsubstituted alkyl, a substituted or unsubstituted alkenyl, a substituted or unsubstituted aryl, a carbonyl, or an alkoxy, and at least one of A₁ and A₂ can be a substituted or unsubstituted aryl; (d) A₃ can be a hydrogen, a substituted or unsubstituted alkyl, a substituted or unsubstituted aryl, a carbonyl, or an alkoxy; (e) R₁—R₃ can be independently a hydrogen, a hydroxyl, a substituted or unsubstituted alkyl, a carbonyl, an alkoxy, a thiol, or an amino; and (f) the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

In some forms, the compounds can have the structure of Formula III:

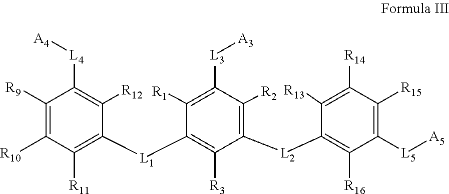

wherein: (a) L₁ and L₂ can be independently a bond (single, double, or triple), a substituted or unsubstituted alkyl, a substituted or unsubstituted alkenyl, a carbonyl, or an alkoxy; (b) L₃-L₅ can be independently a bond (single, double, or triple), a substituted or unsubstituted alkyl, or a carbonyl; (c) A₃-A₅ can be independently a hydrogen, a substituted or unsubstituted alkyl, a substituted or unsubstituted aryl, a carbonyl, or an alkoxy; (d) R₁—R₃ and R₉—R₁₆ can be independently a hydrogen, a hydroxyl, a substituted or unsubstituted alkyl, a carbonyl, an alkoxy, a thiol, or an amino; and (e) the substituents can be as defined above for Formula I.

In some forms, -L₁-A₁ is the same as -L₂-A₂, and the compounds can have the structure of Formula IV:

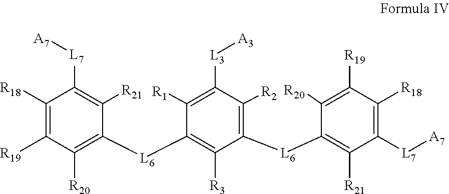

wherein: (a) L₃ and L₇ can be independently a bond (single, double, or triple), a substituted or unsubstituted alkyl, or a carbonyl; (b) L₆ can be a bond (single, double, or triple), a substituted or unsubstituted alkyl, a substituted or unsubstituted alkenyl, a carbonyl, or an alkoxy; (c) A₃ and A₇ can be independently a hydrogen, a substituted or unsubstituted alkyl, a substituted or unsubstituted aryl, a carbonyl, or an alkoxy; (d) R₁—R₃ and R₁₈—R₂₁ can be independently a hydrogen, a hydroxyl, a substituted or unsubstituted alkyl, a carbonyl, an alkoxy, a thiol, or an amino; and (e) the substituents can be as defined above for Formula I.

In some forms, R₁—R₃, R₈—R₁₆, and/or R₁₈—R₂₁ can be independently a hydroxyl, a substituted or unsubstituted alkyl, —OR₄, —SR₅, or —NR₆R₇, R₄—R₇ can be independently a hydrogen or a substituted or unsubstituted alkyl. In some forms, R₁—R₃ can be hydroxyl. In some forms, R₈—R₁₆ and/or R₁₈—R₂₁ can be independently a hydroxyl, a substituted or unsubstituted alkyl, or —OR₄, R₄ can be a substituted or unsubstituted alkyl.

In some forms, -L₃-A₃, -L₄-A₄, -L₅-A₅, and/or -L₇-A₇ can be independently a substituted or unsubstituted alkyl or

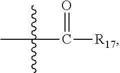

R₁₇ can be a substituted or unsubstituted alkyl, a substituted or unsubstituted aryl, or

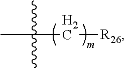

m can be an integer from 0 to 5 and R₂₆ is a carboxylic acid, an ester, an amino, or an amide. In some forms, R₁₇ can be a substituted or unsubstituted C₁-C₅ alkyl, a substituted or unsubstituted C₁-C₄ alkyl, a substituted or unsubstituted C₁-C₃ alkyl, a substituted or unsubstituted C₁-C₂ alkyl, or a substituted or unsubstituted methyl. In some forms, R₁₇ can be a C₆ cycloalkyl. In some forms, R₁₇ can be an unsubstituted phenyl. In some forms, R₂₆ can be an ester.

In some forms, -L₁-A₁ is different from -L₂-A₂, and wherein the compound has the structure of

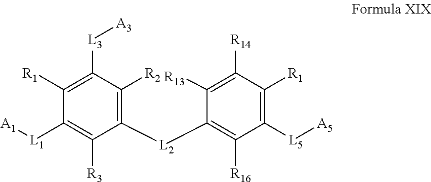

wherein: (a) L₁ and L₂ are independently a single bond, a substituted or unsubstituted alkylene, a substituted or unsubstituted alkenylene, —C(O)—, or an alkoxy; (b) A₁ is a substituted or unsubstituted alkyl, a substituted or unsubstituted alkenyl, a substituted or unsubstituted aryl, a carbonyl, or an alkoxy; (c) R₁—R₃, and/or R₁₃—R₁₆ are independently a hydrogen, a hydroxyl, a substituted or unsubstituted alkyl, —OR₄, —SR₅, or —NR₆R₇, R₄—R₇ are independently a hydrogen or a substituted or unsubstituted alkyl; (d) -L₃-A₃ is

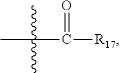

R₁₇ is a substituted or unsubstituted alkyl, a substituted or unsubstituted aryl; and (e) -L₅-A₅ is a hydrogen, a substituted or unsubstituted alkyl, or

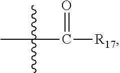

R₁₇ is a substituted or unsubstituted alkyl.

In some forms, -L₁-A₁ is different from -L₂-A₂, and wherein the compound has the structure of

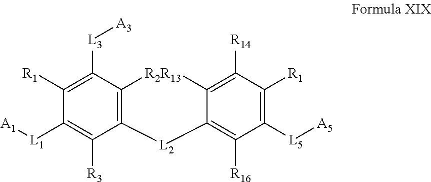

wherein: (a) L₁ and L₂ are independently a substituted or unsubstituted alkylene, or —C(O)—; (b) A₁ is a substituted or unsubstituted aryl, or an C₁-C₆ alkoxy; (c) R₁—R₃ are a hydroxyl; (d) R₁₃—R₁₆ are independently a hydroxyl, a hydrogen, a substituted or unsubstituted alkyl, or —OR₄, R₄ is an C₁-C₆ alkyl; (e) -L₃-A₃ is

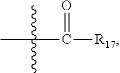

R₁₇ is a substituted or unsubstituted C₁-C₆ alkyl, a substituted or unsubstituted aryl; and (f) -L₅-A₅ is a hydrogen, a substituted or unsubstituted C₁-C₆ alkyl, or

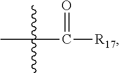

R₁₇ is a substituted or unsubstituted C₁-C₆ alkyl.

In some forms, A₁ is an alkoxy substituted phenyl. In some forms, A₁ is an unsubstituted phenyl. In some forms, -L₃-A₃ is

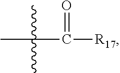

R₁₇ is an unsubstituted phenyl.

In some forms, -L₁-A₁ is different from -L₂-A₂, and wherein the compound has the structure of

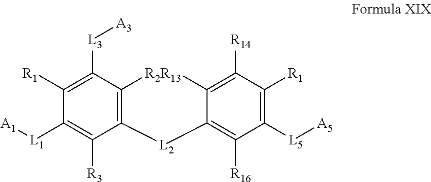

wherein: (a) L₁ and L₂ are independently a substituted or unsubstituted alkylene, or —C(O)—; (b) A₁ is an C₁-C₆ alkoxy; (c) R₁—R₃ are a hydroxyl; (d) R₁₃—R₁₆ are a hydroxyl, a substituted or unsubstituted C₁-C₆ alkyl, or —OR₄, R₄ is a substituted or unsubstituted C₁-C₆ alkyl; (e) -L₃-A₃ is

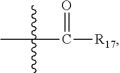

R₁₇ is a substituted or unsubstituted C₁-C₆ alkyl; and (f) -L₅-A₅ is

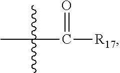

R₁₇ is a substituted or unsubstituted C₁-C₆ alkyl.

In some forms, the compound is not

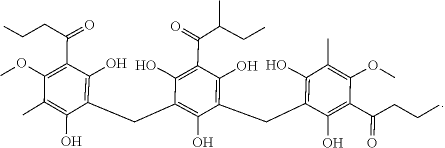 [SMILES: CCCC(=O)c1c(O)c(Cc2c(O)c(Cc3c(O)c(C(=O)CCC)c(OC)c(C)c3CO)c(O)c(C(=O)C(C)CC)c2CO)c(O)c(C)c1OC]

In some forms, the compound can be

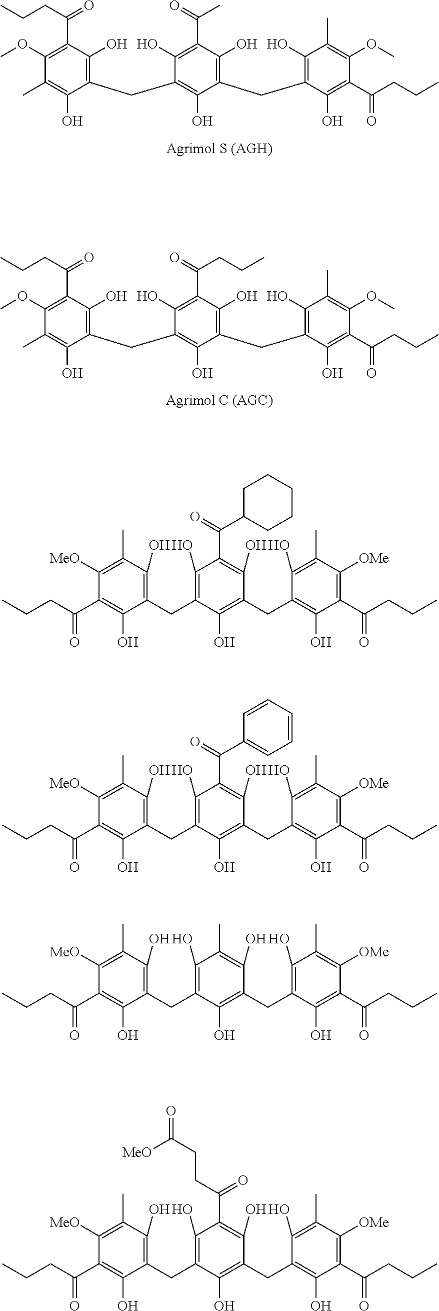

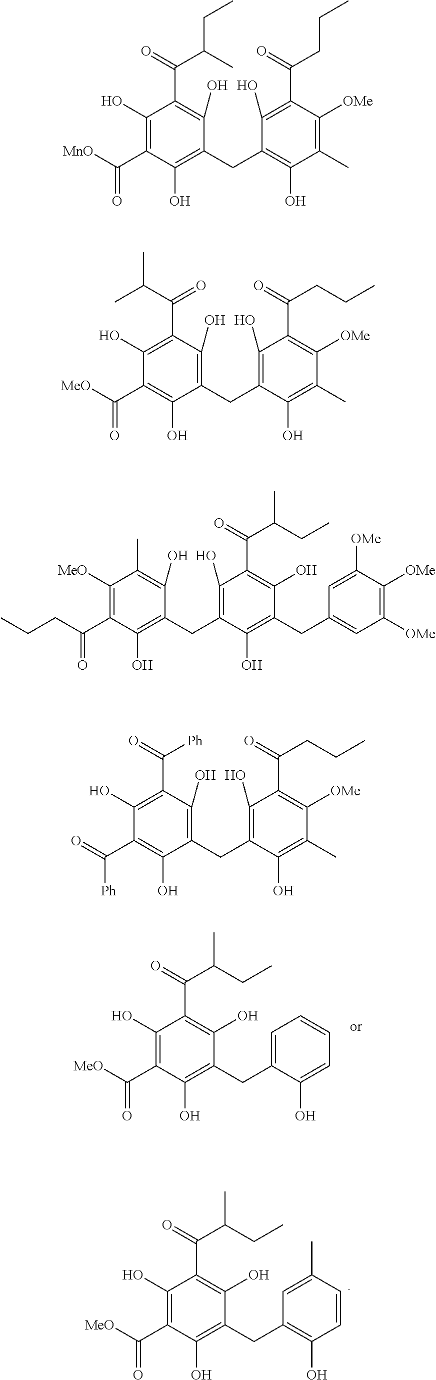

Pharmaceutical formulations containing one or more compounds disclosed herein; and a pharmaceutically acceptable carrier and/or excipient are disclosed. In some forms, the one or more compounds can be in an effective amount to treat a cancer, reduce a cancer, or treat or ameliorate one or more symptoms associated with a cancer in a subject.

In some forms, the effective amount of the one or more compounds can be effective to induce mitochondria dysfunction and/or to inhibit ER to Golgi trafficking in cancer cells in the subject. In some forms, the effective amount of the one or more compounds can be effective to induce apoptosis of cancer cells in the subject. In some forms, the effective amount of the one or more compounds can be effective to inhibit the growth of a tumor by at least 40%, at least 50%, or at least 55% in the subject compared to the same tumor in a control subject.

In some forms, the total concentration of the one or more compounds in the pharmaceutical formulation can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %. In some forms, the pharmaceutical formulation can be in a unit dosage form, wherein the dosage of the compounds can be in a range from about 0.002 mg to about 1 mg, in a range from about 0.006 mg to about 0.6 mg, in a range from about 0.01 mg to about 0.4 mg, in a range from about 0.02 mg to about 0.3 mg, or in a range from about 0.01 mg to about 0.2 mg. In some forms, the pharmaceutical formulation can contain a second active agent, optionally more than one second active agent, optionally the second active agent can be an anticancer agent.

Methods for synthesizing AGB and its derivatives with a high overall yield (i.e. an overall yield ≥7%) have been developed. The method can include (i) heating a reaction mixture at a suitable temperature for a period of time sufficient to form a product comprising the compound, wherein the reaction mixture comprises a solvent, a first reactant having the structure of Formula V,

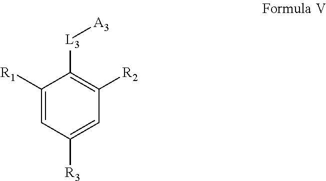

a second reactant having the structure of Formula VI, and

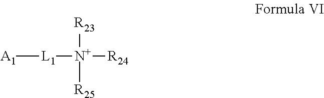

a third reactant having the structure of Formula VII,

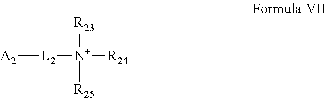

wherein (a) L₁ and L₂ can be independently a bond (single, double, or triple), a substituted or unsubstituted alkyl, a substituted or unsubstituted alkenyl, a carbonyl, or an alkoxy; (b) L₃ can be a bond (single, double, or triple), a substituted or unsubstituted alkyl, or a carbonyl; (c) A₁ and A₂ can be independently a hydrogen, a substituted or unsubstituted alkyl, a substituted or unsubstituted alkenyl, a substituted or unsubstituted aryl, a carbonyl, or an alkoxy, and at least one of A₁ and A₂ is a substituted or unsubstituted aryl; (d) A₃ can be a hydrogen, a substituted or unsubstituted alkyl, a substituted or unsubstituted aryl, a carbonyl, or an alkoxy; (e) R₁—R₃ can be independently a hydrogen, a hydroxyl, a substituted or unsubstituted alkyl, a carbonyl, an alkoxy, a thiol, or an amino; (f) R₂₃—R₂₅ can be independently a hydrogen or a substituted or unsubstituted alkyl; and (g) the substituents can be as defined above for Formula I. The second reactant can be the same as or different from the third reactant.

In some forms, -L₃-A₃ can be

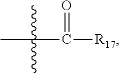

R₁₇ can be an an unsubstituted C₁-C₁₀ alkyl or

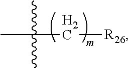

m can be an integer from 0 to 5 and R₂₆ can be a carboxylic acid, an ester, an amino, or an amide.

In some forms, A₁ can be

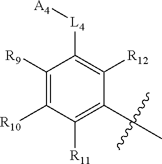

### and A₂ can be

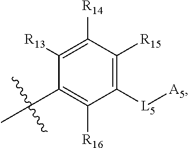

wherein: (a) L₄ and L₅ can be independently a bond (single, double, or triple), a substituted or unsubstituted alkyl, or a carbonyl; (b) A₄ and A₅ can be independently a hydrogen, a substituted or unsubstituted alkyl, a substituted or unsubstituted aryl, a carbonyl, or an alkoxy; (c) R₉—R₁₆ can be independently a hydrogen, a hydroxyl, a substituted or unsubstituted alkyl, a carbonyl, an alkoxy, a thiol, or an amino; and (d) the substituents can be as defined above for Formula I.

In some forms, the second reactant can be the same as the third reactant, having the structure of:

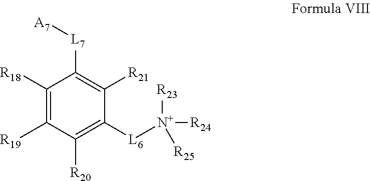

wherein: (a) L₆ can be a bond (single, double, or triple), a substituted or unsubstituted alkyl, a substituted or unsubstituted alkenyl, a carbonyl, or an alkoxy; (b) L₇ can be a bond (single, double, or triple), a substituted or unsubstituted alkyl, or a carbonyl; (c) A₇ can be a hydrogen, a substituted or unsubstituted alkyl, a substituted or unsubstituted aryl, a carbonyl, or an alkoxy; (d) R₁₈—R₂₁ can be independently a hydrogen, a hydroxyl, a substituted or unsubstituted alkyl, a carbonyl, an alkoxy, a thiol, or an amino; (e) R₂₃—R₂₅ can be independently a hydrogen or a substituted or unsubstituted alkyl; and (f) the substituents can be as defined above for Formula I.

In some forms, the method can further include (i-a) converting a pre-reactant having the structure of Formula VIII′ to the reactant having the structure of Formula VIII:

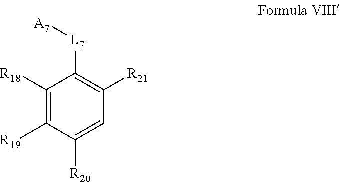

wherein: (a) L₇ can be a bond (single, double, or triple), a substituted or unsubstituted alkyl, or a carbonyl; (b) A₇ can be a hydrogen, a substituted or unsubstituted alkyl, a substituted or unsubstituted aryl, a carbonyl, or an alkoxy; (c) R₁₈—R₂₁ can be independently a hydrogen, a hydroxyl, a substituted or unsubstituted alkyl, a carbonyl, an alkoxy, a thiol, or an amino; and (d) the substituents can be as defined above for Formula I.

In some forms, R₁₉ can be a substituted or unsubstituted alkyl, such as an unsubstituted methyl. In some forms, R₁₈ can be —OR₂₂, each R₂₂ can be hydrogen or a substituted or unsubstituted alkyl. In some forms, R₂₀ and R₂₁ can be hydroxyl. In some forms, R₁₉ can be a substituted or unsubstituted alkyl, such as an unsubstituted methyl; R₁₈ can be —OR₂₂, each R₂₂ can be hydrogen or a substituted or unsubstituted alkyl; and R₂₀ and R₂₁ can be hydroxyl. In some forms, -L₇-A₇ can be

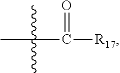

R₁₇ can be an unsubstituted C₁-C₁₀alkyl, an unsubstituted C₁-C₈ alkyl, an unsubstituted C₁-C₆ alkyl, an unsubstituted C₁-C₄ alkyl, an unsubstituted C₁-C₃ alkyl, or

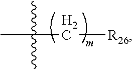

m can be an integer from 0 to 5 and R₂₆ can be a carboxylic acid, an ester, an amino, or an amide. In some forms, -L₇-A₇ can be

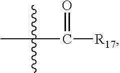

R₁₇ can be an unsubstituted C₁-C₁a alkyl, an unsubstituted C₁-C₈ alkyl, an unsubstituted C₁-C₆ alkyl, an unsubstituted C₁-C₄ alkyl, or an unsubstituted C₁-C₃ alkyl.

The method can further include (i-b) converting a suitable starting material to the pre-reactant having the structure of Formula VIII′.

In some forms of step (i-b), the method can include (i-b1) converting a first starting material having the structure of Formula X to a first intermediate having the structure of Formula IX and (i-b2) converting the first intermediate having the structure of Formula IX to the pre-reactant having the structure of Formula VIII′:

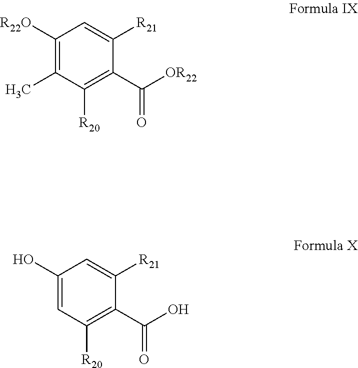

wherein: (a) R₂₀ and R₂₁ can be independently a hydrogen, a hydroxyl, a substituted or unsubstituted alkyl, a carbonyl, an alkoxy, a thiol, or an amino; (b) R₂₂ can be a hydrogen or a substituted or unsubstituted alkyl; and (c) the substituents can be as defined above for Formula I. In some forms, R₂₀ and R₂₁ can be hydroxyl.

In some forms, the conversion from the first starting material of Formula X to the first intermediate of Formula IX (i.e. step (i-b1)) can include a methylation step and/or an acylation step. In some forms, the conversion from the first starting material of Formula X to the first intermediate of Formula IX can include a methylation step and an acylation step. In some forms, the methylation step can be performed prior to the acylation step.

In some forms, in the methylation step of step (i-b1), the starting material can be converted a methylated compound having the structure of Formula XI′:

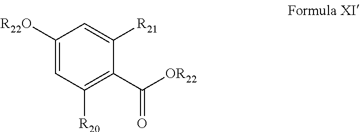

wherein (a) R₂₀ and R₂₁ can be independently a hydrogen, a hydroxyl, a substituted or unsubstituted alkyl, a carbonyl, an alkoxy, a thiol, or an amino; (b) R₂₂ can be a hydrogen or a substituted or unsubstituted alkyl; and (c) the substituents can be as defined above for Formula I.

In some forms, in the acylation step of step (i-b1), the methylated compound of Formula XI′ can be converted to an acylated compound having the structure of Formula XI:

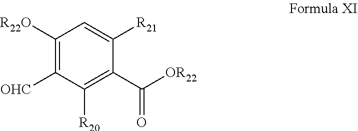

wherein (a) R₂₀ and R₂₁ can be independently a hydrogen, a hydroxyl, a substituted or unsubstituted alkyl, a carbonyl, an alkoxy, a thiol, or an amino; (b) R₂₂ can be a hydrogen or a substituted or unsubstituted alkyl; and (c) the substituents can be as defined above for Formula I.

In some forms, the conversion from the starting material of Formula X to the intermediate of Formula IX further includes a reduction step, in which the acylated compound having the structure of Formula XI can be converted to the intermediate having the structure of Formula IX. In some forms, the coversion of the acylated compound of Formula XI to the intermediate of Formula IX is via a Vilsmeier-Haack reaction.

In some forms, the conversion of the first intermediate of Formula IX to the pre-reactant of Formula VIII′ (i.e. step (i-b2)) can include an acylation step and a decarboxylation step, preformed sequentially. Exemplary steps in the conversion from the first intermediate of Formula IX to the pre-reactant of Formula VIII′ are shown below.

In alternative forms of step (i-b) (converting a suitable starting material to the pre-reactant having the structure of Formula VIII′), the method can include (i-b1′) converting phloroglucinol (as the starting material) to a second intermediate having the structure of Formula XVII and (i-b2′) converting the second intermediate having the structure of Formula XVII to the pre-reactant having the structure of Formula VIII′:

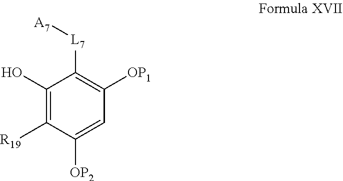

wherein: (a) L₇ can be a bond (single, double, or triple), a substituted or unsubstituted alkyl, or a carbonyl; (b) A₇ can be a hydrogen, a substituted or unsubstituted alkyl, a substituted or unsubstituted aryl, a carbonyl, or an alkoxy; (c) R₁₉ can be a hydrogen, a hydroxyl, a substituted or unsubstituted alkyl, a carbonyl, an alkoxy, a thiol, or an amino; (d) P₁ and P₂ can be independently a protection group, such as a methoxymethyl ether, tetrahydropyranyl ether, butyl ether, allyl ether, acetonide, benzylidene acetal, acetic acid esters, pivalic acid ester, tert-butyldimethylsilyl ethers, tert-butyldibenzylsilyl ethers, benzyl ether, benzoic acid ester, benzoate ester, benzoate; and (e) the substituents can be as defined above for Formula I. In some forms, R₁₉ can be a substituted or unsubstituted alkyl, such as an unsubstituted C₁-C₁₀ alkyl, an unsubstituted C₁-C₈ alkyl, an unsubstituted C₁-C₆ alkyl, an unsubstituted C₁-C₄ alkyl, an unsubstituted C₁-C₃ alkyl, or an unsubstituted C₁-C₂ alkyl, such as an unsubstituted methyl.

In some forms, the conversion from phloroglucinol to the second intermediate of Formula XVII (i.e. step (i-b1′)) can include an acylation step. In some forms, the conversion from phloroglucinol to the second intermediate of Formula XVII can include an oxidation step, a reduction step, and an acylation step, performed sequentially. In some forms, the conversion from phloroglucinol to the second intermediate of Formula XVII can include an oxidation step, a reduction step, an acylation step, and a protection step, performed sequentially. Exemplary steps in the conversion from phloroglucinol to the second intermediate of Formula XVII are shown below.

In some forms, the conversion from the second intermediate of Formula XVII to the pre-reactant of Formula VIII′ (i.e. step (i-b2′)) can include an acylation step and optionally a deprotection step. In some forms, in the acylation step of step (i-b2′), the second intermediate of Formula XVII can be converted to an acylated compound having the structure of Formula XVIII:

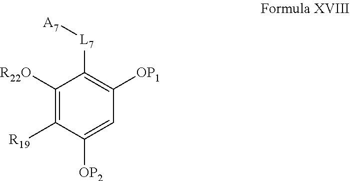

wherein: (a) L₇ can be a bond (single, double, or triple), a substituted or unsubstituted alkyl, or a carbonyl; (b) A₇ can be a hydrogen, a substituted or unsubstituted alkyl, a substituted or unsubstituted aryl, a carbonyl, or an alkoxy; (c) R₁₉ can be a hydrogen, a hydroxyl, a substituted or unsubstituted alkyl, a carbonyl, an alkoxy, a thiol, or an amino; (d) R₂₂ can be a hydrogen or a substituted or unsubstituted alkyl; (e) P₁ and P₂ can be independently a protection group as described above for Formula XVII; and (f) the substituents can be as defined above for Formula I.

In some forms, the first reactant can be

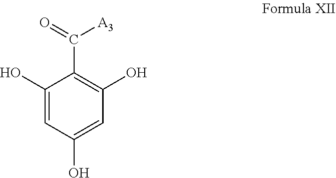

the second reactant can be the same as the third reactant, having the structure of Formula XIII:

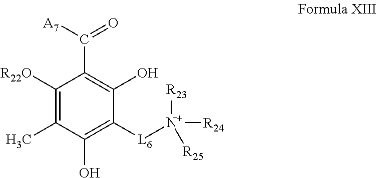

wherein: (a) A₃ and A₇ can be independently a hydrogen, a substituted or unsubstituted alkyl, a substituted or unsubstituted aryl, a carbonyl, or an alkoxy; (b) L₆ can be a bond (single, double, or triple), a substituted or unsubstituted alkyl, a substituted or unsubstituted alkenyl, a carbonyl, or an alkoxy; (c) R₂₂ can be a hydrogen or a substituted or unsubstituted alkyl; (d) R₂₃—R₂₅ can be independently a hydrogen or a substituted or unsubstituted alkyl; and (e) the substituents can be as defined above for Formula I.

In some forms, the solvent in the reaction mixture can be dichloromethane, dimethyl sulfate, dimethyl sulfoxide, dimethyl formamide, butyryl chloride, dichloroethane, nitrobenzene, dioxane, 2-methylbutyryl chloride, ethyl acetate, ethyl lactate, acetone, 1-butanol, 1-propanol, 2-propanol, ethanol, isopropyl acetate, methanol, methyl ethyl ketone, t-butanol, tetrahydrofuran, 2-methyl tetrahydrofuran, acetonitrile, or toluene, or a combination thereof, or a mixture with water thereof. In some forms, the reaction mixture can be heated at a temperature of up to 130° C., in a range from about 100° C. to about 130° C., from about 100° C. to about 125° C., from about 100° C. to about 120° C., at 1 atm, such as about 110° C., for a time period in a range from about 30 mins to about 4 hours, from about 30 mins to about 3.5 hours, from about 30 mins to about 3 hours, from about 30 mins to about 2.5 hours, from about 1 hour to about 4 hours, from about 1 hour to about 3 hours, or from about 1 hour to about 2 hours, optionally under reflux. In some forms, the method can further include mixing the first reactant, the second reactant, the third reactant, and the solvent to form the reaction mixture prior to step (i) and/or purifying the product containing the prodrug subsequent to step (i).

In some forms, the AGB or AGB derivative formed using the disclosed method can have an overall yield of at least 7%, at least 10%, at least 15%, at least 20%, at least 25%, or at least 30%.

Methods of using the synthetic AGB and AGB derivatives for treating a cancer, reducing a cancer, or treating or ameliorating one or more symptoms associated with a cancer in a subject are disclosed. Generally, the method includes (i) administering to the subject the pharmaceutical formulation containing one or more the disclosed compounds, wherein step (i) occurs one or more times. In some forms, the subject can be a mammal. In some forms, the pharmaceutical formulation can be administered by oral administration, parenteral administration, inhalation, mucosal administration, topical or a combination thereof. In some forms, the cancer being treated using the disclosed method is non-small-cell lung cancer. In some forms, the method can further include administering to the subject a second active agent, optionally more than one second active agent, prior to, during, and/or subsequent to step (i), and optionally the second active agent can be an anticancer agent.

In some forms, the method can include only a single administration of the pharmaceutical formulation, wherein the pharmaceutical formulation contains an effective amount of the compounds to inhibit the growth of a tumor by at least 40%, at least 50%, or at least 55% in the subject compared to the same tumor in a control subject. In some forms, the method can include more than one step of administering to the subject the pharmaceutical formulation, wherein following all of the administration steps an effective amount of the compounds to inhibit the growth of a tumor by at least 40%, at least 50%, or at least 55% in the subject compared to the same tumor in a control subject is administered to the subject. In some forms, the effective amount of the compounds administered to the subject can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 3 mg/kg.

Methods of using the synthetic AGB and AGB derivatives for treating cancer cells and/or cancer stem cells in a subject in need thereof are also disclosed. Generally, the method includes (i) administering to the subject the pharmaceutical formulation containing one or more the disclosed compounds, wherein step (i) occurs one or more times.

In some forms, the compounds can have an IC₅₀ value against test cancer cells lower than IC₅₀ value of the same compound against non-cancerous cells, tested under the same conditions. In some forms, the test cancer cells can be MDA-MB-231, MCF-7, HepG2, Hela, AGS, HTC116, SW480, SUNE-1, H460, HCC827, H1650, or A549, or a combination thereof. In some forms, the non-cancerous cells can be CCD-19Lu.

In some forms, the method can include only a single administration of the pharmaceutical formulation, wherein the pharmaceutical formulation contains an effective amount of the compounds to induce mitochondria dysfunction and/or to inhibit ER to Golgi trafficking in cancer cells in the subject, compared to the subject before administered with the pharmaceutical formulation. In some forms, the method can include more than one step of administering to the subject the pharmaceutical formulation, wherein following all of the administration steps an effective amount of the compounds to induce mitochondria dysfunction and/or to inhibit ER to Golgi trafficking in cancer cells in the subject, compared to the subject before administered with the pharmaceutical formulation, is administered in the subject.

In some forms, the method can include only a single administration of the pharmaceutical formulation, wherein the pharmaceutical formulation contains an effective amount of the compounds to induce apoptosis of cancer cells in the subject, compared to the subject before administered with the pharmaceutical formulation. In some forms, the method can include more than one step of administering to the subject the pharmaceutical formulation, wherein following all of the administration steps an effective amount of the compounds to induce apoptosis of cancer cells in the subject, compared to the subject before administered with the pharmaceutical formulation, is administered in the subject.

### BRIEF DESCRIPTION OF THE DRAWINGS

FIG. 1A is a bar graph showing the IC50 values of AGB against indicated cell lines. Cells were treated with AGB at various concentrations for 48 h and processed for MTT assay. The IC₅₀ values were half maximal inhibitory concentration that determined by the fitting curve using the equation from GraphPad Prism 8. FIG. 1B is a bar graph showing the inhibition of colony formation on NCI-H460 cells by AGB. Cells were treated with indicated concentrations of AGB for 8 h. Medium with AGB was then aspirated and cells were re-seeded into a new 12-well cell culture plate. After 14 days, cells were fixed by ethanol followed by staining using crystal violet. Colony numbers were counted and normalized to vehicle control group. Colonies with more than 50 cells were considered. **, p<0.01; ***, p<0.001. FIG. 1C shows cell migration using the wound healing assay. The cell monolayer of NCI-H460 cells were scratched by sterile 200 μl pipette tip, followed by treatment with DMSO (vehicle) or indicated concentrations of AGB for 16 h and 32 h. The pictures were captured at 100× magnification in the bright field. FIG. 1D is a bar graph showing the inhibition of self-renewal and growth of CSCs by AGB. The numbers of formed tumorsphere were calculated and normalized to the control group of primary passage. Three independent experiments were performed and data were shown as the mean±SD, *, p<0.05, ^{##}, p<0.01. Scare bar: 200 m. FIG. 1E is a graph showing the cell viability of CSCs determined by MTT assay. The absorbance of each well was normalized to control wells and data were shown as percentage of cell viabilities. The response curve of the cell viabilities was plotted by GraphPad Prism and the curve was fitted by the Boltzmann equation. FIGS. 1F-1G shows images of patient-derived organoids from metastatic lung cancer tumor after AGB treatment at different concentrations for 72 h (FIG. 1F) and the cell viability determined using CellTiter Glo™ at 72 hours post-treatment (FIG. 1G). FIGS. 1H-1J are graphs showing relative tumor volume curves of each treatment group: A549 xenograft models (FIG. 1H), NCI-H460 xenograft models (FIG. 1I), and NCI-H460 cancer stem like xenograft models using NOD/SCID mice (FIG. 1J) (n=5). The tumor volume was normalized to that of the first day. The data are presented as means±SEM; *, p<0.05. FIG. 1K shows the HE-staining images of important organs (kidney and liver in this experiment) and tumor samples with or without AGB treatment.

FIGS. 2A-2C are graphs showing results of ProTargetMiner analysis of differential expression proteins induced by AGB. FIG. 2A shows the orthogonal partial least squares discriminant analysis (OPLS-DA) model used to contrast AGB proteome signature against all other anticancer drugs in library. Three classes of proteins were highlighted as the most specific regulations induced by AGB: the mitochondrial ribosome proteins (MRPs), NADH dehydrogenases, and the vesicle transporter proteins. FIG. 2B shows the k-means clustering by STRING of the most specific regulation proteins highlighted by OPLS-DA analysis. Top-30 from those down-regulated and up-regulated proteins were submitted for analysis. Three main clusters of interactions were obtained and grouped into three different colors: proteins in cluster A belong to mitochondrial ribosomal proteins; Cluster B contains mostly vesicle transport proteins that regulate ER to Golgi trafficking; and Cluster C represents proteins for biological function of the mitochondrial respiratory chain complex I assembly. FIG. 2C shows the GO analysis of biological processes affected upon treatments with AGB.

FIG. 3A is a graph showing the change in fluoresence intensity following AGB treatment for different indicated times, determined by a flow cytometry. FIG. 3B shows images of the ultrastructure of mitochondria treated with AGB and the cristae of mitochondria (displayed a disruption).

FIG. 4 is a graph showing AGB induced apoptosis in NCI-H460 cells by the Annexin-V/PI double-staining assay. After treating with AGB for 24 h and 36 h, respectively, cells were stained with annexin V-FITC and propidium iodide and analyzed by flow cytometry.

FIG. 5A is a volcano plot (ΔTm, p-value) for TPP data obtained from NCI-H460 cells with 4 μM AGB treatment for 1 h. The criteria were set as ΔTₘ>2° C. and p<0.05. The proteins of CS, TALDO1, and HSPD1 were highlighted and indicated in the Figure. FIG. 5B is a graph showing results of the tryptophan fluorescence quenching assay for the direct interaction between AGB and three target proteins. Upper, tryptophan fluorescence titration of recombinant proteins with AGB. The tryptophan fluorescence of target proteins excited at 260 nm showed decreased intensity after titrating different concentrations of AGB. For CS, its fluorescence decrease was saturated when AGB concentrations increased to about 32 μM. Lower, the normalized tryptophan intensities at an emission of 320 nm were plotted as a function of total added concentration of AGB. The dissociation constants (K_{d}) between AGB and three proteins were then calculated by fitting this plot with Stern-Volmer Equation. FIG. 5C is a bar graph showing that AGB decreased the refolding activity of MDH in a dose dependent manner. *, p<0.05; **, p<0.01. FIG. 5D is a graph showing the fluoresence intensity of EGFP and Mito-RFP after the treatment with different concentrations of AGB. The fluorescence intensity was presented as percentage relative to control groups. FIG. 5E is a bar graph showing the relative CS activity after treatment with different concentrations of AGB. AGB decreased the CS catalytic activity in a dose dependent manner. *, p<0.05; **, p<0.01. FIG. 5F is a bar graph showing that AGB decreased the amount of phosphate pentose pathway intermediates in cells. FIG. 5G is a bar graph showing that AGB decreased the intracellular level of NADPH. **, p<0.01. (K) Docking results for AGB and HSP60, CS, and TALDO1.

FIGS. 6A-6M show the spectrum of some AGB derivatives.

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

The term “alkyl” as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF₃, —CH₂—CF₃, —CCl₃); —CN; —NCOCOCH₂CH₂; —NCOCOCHCH; and —NCS; and combinations thereof. The term “alkyl” also includes “heteroalkyl.”

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom. Suitable heteroatoms include, but are not limited to, 0, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C═C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

The term “aryl” as used herein is any C₅-C₂₆ carbon-based aromatic group, heteroaromatic, fused aromatic, or fused heteroaromatic. For example, “aryl,” as used herein can include 5-, 6-, 7-, 8-, 9-, 10-, 14-, 18-, and 24-membered single-ring aromatic groups, including, but not limited to, benzene, naphthalene, anthracene, phenanthrene, chrysene, pyrene, corannulene, coronene, etc. “Aryl” further encompasses polycyclic ring systems having two or more cyclic rings in which two or more carbons are common to two adjoining rings (i.e., “fused aromatic rings”), wherein at least one of the rings is aromatic, e.g., the other cyclic ring or rings can be cycloalkyls, cycloalkenyls, cycloalkynyls, aryls and/or heterocycles. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “substituted aryl” refers to an aryl group, wherein one or more hydrogen atoms on one or more aromatic rings are substituted with one or more substituents. Such substituents can be any substituents described above, e.g., halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₃₀-membered aromatic, fused aromatic, biaromatic ring systems, or combinations thereof, in which one or more carbon atoms on one or more aromatic ring structures have been substituted with a heteroatom. Suitable heteroatoms include, but are not limited to, oxygen, sulfur, and nitrogen. Broadly defined, “heteroaryl,” as used herein, includes 5-, 6-, 7-, 8-, 9-, 10-, 14-, 18-, and 24-membered single-ring aromatic groups that may include from one to four heteroatoms, for example, pyrrole, furan, thiophene, imidazole, oxazole, thiazole, triazole, tetrazole, pyrazole, pyridine, pyrazine, pyridazine and pyrimidine, and the like. The heteroaryl group may also be referred to as “aryl heterocycles” or “heteroaromatics.” “Heteroaryl” further encompasses polycyclic ring systems having two or more rings in which two or more carbons are common to two adjoining rings (i.e., “fused rings”) wherein at least one of the rings is heteroaromatic, e.g., the other cyclic ring or rings can be cycloalkyls, cycloalkenyls, cycloalkynyls, aryls, heterocycles, or combinations thereof. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents. Such substituents can be any substituents described above, e.g., halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a chemical moiety that includes two or more aryls, heteroaryls, and combinations thereof. The aryls, heteroaryls, and combinations thereof, are fused, or linked via a single bond, ether, ester, carbonyl, amide, sulfonyl, sulfonamide, alkyl, azo, and combinations thereof. For example, a “polyaryl” can be polycyclic ring systems having two or more cyclic rings in which two or more carbons are common to two adjoining rings (i.e., “fused aromatic rings”), wherein two or more of the rings are aromatic. When two or more heteroaryls are involved, the chemical moiety can be referred to as a “polyheteroaryl.”

The term “substituted polyaryl” refers to a polyaryl in which one or more of the aryls, heteroaryls are substituted, with one or more substituents. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. When two or more heteroaryls are involved, the chemical moiety can be referred to as a “substituted polyheteroaryl.”

The term “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O— arakyl, —O-aryl, —O-heteroaryl, —O-polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

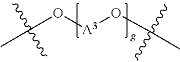

where A³, A², and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —OR^{v} wherein R^{v} is C₆H₅ (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

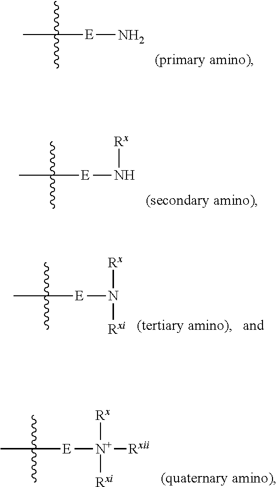

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R″′; R″′ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure.

The terms “amide” or “amido” are used interchangeably, refer to both “unsubstituted amido” and “substituted amido” and are represented by the general formula:

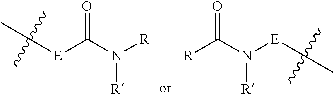

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R″′, or R and R′ taken together with the N atom to which they are attached complete a heterocycle having from 3 to 14 atoms in the ring structure; R″′ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed.

“Carbonyl,” as used herein, is art-recognized and includes such moieties as can be represented by the general formula:

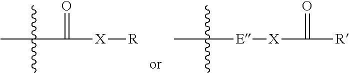

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “substituted carbonyl” refers to a carbonyl, as defined above, wherein one or more hydrogen atoms in R, R′ or a group to which the moiety

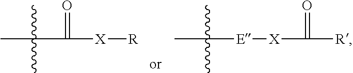

is attached, are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “carboxyl” is as defined above for carbonyl and is defined more specifically by the formula —R^{iv}COOH, wherein R^{iv} is a substituted or unsubstituted alkyl, a substituted or unsubstituted alkenyl, a substituted or unsubstituted alkynyl, a substituted or unsubstituted heterocyclyl, a substituted or unsubstituted alkylaryl, a substituted or unsubstituted arylalkyl, a substituted or unsubstituted aryl, a substituted or unsubstituted polyaryl, a substituted or unsubstituted polyheteroaryl, or a substituted or unsubstituted heteroaryl.

The term “substituted carboxyl” refers to a carboxyl, as defined above, wherein one or more hydrogen atoms in R^{iv} are substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “phosphanyl” is represented by the formula

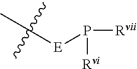

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R″′, or R^{vi} and R^{vii} taken together with the P atom to which they are attached complete a heterocycle having from 3 to 14 atoms in the ring structure; R″′ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “phosphonium” is represented by the formula

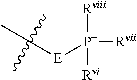

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vi}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R″′, or R^{vi}, R^{vii}, and R^{viii} taken together with the P⁺ atom to which they are attached complete a heterocycle having from 3 to 14 atoms in the ring structure; R″′ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “phosphonyl” is represented by the formula

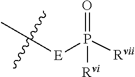

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R″′, or R^{vi} and R^{vii} taken together with the P atom to which they are attached complete a heterocycle having from 3 to 14 atoms in the ring structure; R″′ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “substituted phosphonyl” represents a phosphonyl in which E, R^{vi} and R^{vii} are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “sulfinyl” is represented by the formula

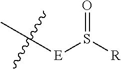

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R″′, or E and R taken together with the S atom to which they are attached complete a heterocycle having from 3 to 14 atoms in the ring structure; R″′ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “sulfonyl” is represented by the formula

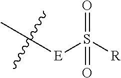

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″′, or E and R taken together with the S atom to which they are attached complete a heterocycle having from 3 to 14 atoms in the ring structure; R″′ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “substituted sulfonyl” represents a sulfonyl in which E, R, or both, are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl.

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art.

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R″′, R″′ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art.

The term “sulfamoyl” refers to a sulfonamide or sulfonamide represented by the formula

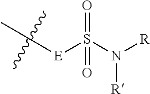

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″′, or R and R′ taken together with the N atom to which they are attached complete a heterocycle having from 3 to 14 atoms in the ring structure; R″′ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “silyl group” as used herein is represented by the formula —SiRR′R,″ where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above.

The term “alkylthio” refers to an alkyl group, as defined above, having a sulfur radical attached thereto. The “alkylthio” moiety is represented by —S-alkyl. Representative alkylthio groups include methylthio, ethylthio, and the like. The term “alkylthio” also encompasses cycloalkyl groups having a sulfur radical attached thereto.

The term “substituted alkylthio” refers to an alkylthio group having one or more substituents replacing one or more hydrogen atoms on one or more carbon atoms of the alkylthio backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “phenylthio” is art recognized, and refers to —S—C₆H₅, i.e., a phenyl group attached to a sulfur atom.

The term “substituted phenylthio” refers to a phenylthio group, as defined above, having one or more substituents replacing a hydrogen on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

“Arylthio” refers to —S-aryl or —S-heteroaryl groups, wherein aryl and heteroaryl as defined herein.

The term “substituted arylthio” represents —S-aryl or —S-heteroaryl, having one or more substituents replacing a hydrogen atom on one or more ring atoms of the aryl and heteroaryl rings as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

### II. Compositions

Synthetic agrimol B (AGB) and derivatives thereof have been developed. The synthetic compunds disclosed herein have anticancer properties and should be suitable for use in the treatment or amelioration of the symptoms of multiple types of cancers. For example, these compunds are suitable for treating or ameliorating symptom(s) of non-small-cell lung cancer. Without being bound to any theory, a dimeric acylphloroglucinol and acyl groups on the ring structure contributes to the antiancer activity of these compounds, rendering them useful in binding with certain biological targets, thereby inducing apoptosis of cancer cells. For example, it is believed that the mechanism of AGB's and its derivatives' anticancer activities includes mitochondrial dysfunction and/or blocking of ER-Golgi trafficking, inducing apoptosis of cancer cells. As exemplified in the Examples, the biological targets of AGB were identified as citrate synthase (CS), transaldolase 1 (TALDO1), and heat shock protein 60 (HSP60) by an unbiased thermal proteome profiling method.

Pharmaceutical formulations containing the compounds are also disclosed.

### A. Agrimol B and Derivatives Thereof

The AGB and derivatives thereof (also referred to herein as “compounds”) can have the structures of Formula I:

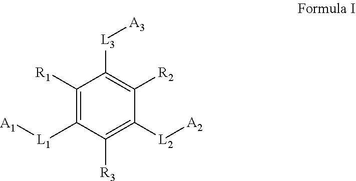

wherein: (a) L₁ and L₂ can be independently a bond (single, double, or triple), a substituted or unsubstituted alkyl, a substituted or unsubstituted alkenyl, a carbonyl, or an alkoxy; (b) L₃ can be a bond (single, double, or triple), a substituted or unsubstituted alkyl, or a carbonyl; (c) A₁ and A₂ can be independently a hydrogen, a substituted or unsubstituted alkyl, a substituted or unsubstituted alkenyl, a substituted or unsubstituted aryl, a carbonyl, or an alkoxy, and at least one of A₁ and A₂ is a substituted or unsubstituted aryl; (d) A₃ can be a hydrogen, a substituted or unsubstituted alkyl, a substituted or unsubstituted aryl, a carbonyl, or an alkoxy; (e) R₁—R₃ can be independently a hydrogen, a hydroxyl, a substituted or unsubstituted alkyl, a carbonyl, an alkoxy, a thiol, or an amino; and (f) the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

In some forms, the AGB and derivatives thereof described herein, such as one having the structure of Formula I, contain at least two aryl groups, i.e., containing two, three or more aryl groups, such as containing three or four aryl groups, e.g. containing three aryl groups.

In some forms, the compound can have the structure of Formula III:

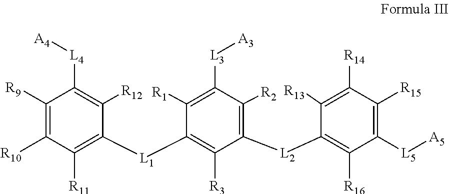

wherein: (a) L₁ and L₂ can be independently a bond (single, double, or triple), a substituted or unsubstituted alkyl, a substituted or unsubstituted alkenyl, a carbonyl, or an alkoxy; (b) L₃-L₅ can be independently a bond (single, double, or triple), a substituted or unsubstituted alkyl, or a carbonyl; (c) A₃-A₅ can be independently a hydrogen, a substituted or unsubstituted alkyl, a substituted or unsubstituted aryl, a carbonyl, or an alkoxy; (d) R₁—R₃ and R₉—R₁₆ can be independently a hydrogen, a hydroxyl, a substituted or unsubstituted alkyl, a substituted or unsubstituted aryl, a carbonyl, an alkoxy, a thiol, or an amino; and (e) the substituents can be as defined above for Formula I. In some forms, A₃-A₅ and/or R₁—R₃ and R₉—R₁₆ are not substituted or unsubstituted aryl groups. In some forms, A₃-A₅, R₁—R₃, and R₉—R₁₆ are not substituted or unsubstituted aryl groups.

For any of Formulae I, Ia, and III, -L₁-A₁ can be the same as or different from -L₂-A₂. For any of Formulae I, Ia, and III, -L₁-A₁ can be the same as -L₂-A₂. In some forms, the compound can have the structure of Formula IV:

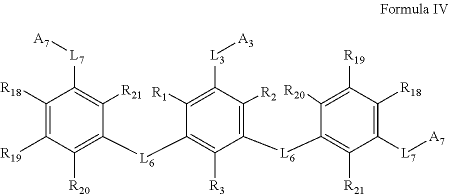

wherein: (a) L₃ and L₇ can be independently a bond (single, double, or triple), a substituted or unsubstituted alkyl, or a carbonyl; (b) L₆ can be a bond (single, double, or triple), a substituted or unsubstituted alkyl, a substituted or unsubstituted alkenyl, a carbonyl, or an alkoxy; (c) A₃ and A₇ can be independently a hydrogen, a substituted or unsubstituted alkyl, a substituted or unsubstituted aryl, a carbonyl, or an alkoxy; (d) R₁—R₃ and R₁₈—R₂₁ can be independently a hydrogen, a hydroxyl, a substituted or unsubstituted alkyl, a substituted or unsubstituted aryl, a carbonyl, an alkoxy (e.g. methoxy, ethoxy, aryloxy, benzoether, etc.), a thiol, or an amino; and (e) the substituents can be as defined above for Formula I. In some forms, A₃ and A₇ and/or R₁—R₃ and R₁₈—R₂₁ are not substituted or unsubstituted aryl groups. In some forms, A₃, A₇, R₁—R₃ and R₁₈—R₂₁ are not substituted or unsubstituted aryl groups.

In some forms of Formula IV, R₁₈—R₂₁ can be independently a hydrogen, a hydroxyl, a substituted or unsubstituted alkyl, a carbonyl, or an alkoxy (e.g. methoxy, ethoxy, etc.).

In some forms, -L₁-A₁ can be different from -L₂-A₂. In some forms, the compound can have the structure of Formula XIX:

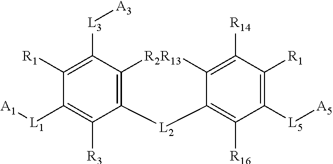

wherein: (a) L₁ and L₂ are independently a single bond, a substituted or unsubstituted alkylene, a substituted or unsubstituted alkenylene, —C(O)—, or an alkoxy; (b) A₁ is a substituted or unsubstituted alkyl, a substituted or unsubstituted alkenyl, a substituted or unsubstituted aryl, a carbonyl, or an alkoxy; (c) R₁—R₃, and/or R₁₃—R₁₆ are independently a hydrogen, a hydroxyl, a substituted or unsubstituted alkyl, —OR₄, —SR₅, or —NR₆R₇, R₄—R₇ are independently a hydrogen or a substituted or unsubstituted alkyl; (d) -L₃-A₃ is

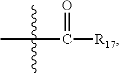

R₁₇ is a substituted or unsubstituted alkyl, a substituted or unsubstituted aryl; and (e) -L₅-A₅ is a hydrogen, a substituted or unsubstituted alkyl, or

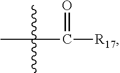

R₁₇ is a substituted or unsubstituted alkyl.

For any of Formulae I, Ia, II, III, IV and XIX, R₁—R₃, R₈—R₁₆, and/or R₁₈—R₂₁ can be independently a hydrogen, a hydroxyl, a substituted or unsubstituted alkyl, —OR₄, —SR₅, or —NR₆R₇, R₄—R₇ can be independently a hydrogen or a substituted or unsubstituted alkyl.

For any of Formulae I, Ia, II, III, IV and XIX, R₁—R₃ can be hydroxyl.

For any of Formulae Ta, II, III, IV, and XIX, R₈—R₁₆ and/or R₁₈—R₂₁ can be independently a hydrogen, a hydroxyl, a substituted or unsubstituted alkyl, or —OR₄, R₄ can be a substituted or unsubstituted alkyl. In some forms, R₄ can be a substituted or unsubstituted C₁-C₆ alkyl, a substituted or unsubstituted C₁-C₅ alkyl, a substituted or unsubstituted C₁-C₄ alkyl, a substituted or unsubstituted C₁-C₃ alkyl, a substituted or unsubstituted C₁-C₂ alkyl, or a substituted or unsubstituted methyl. In some forms, R₄ can be an unsubstituted C₁-C₅ alkyl, an unsubstituted C₁-C₄ alkyl, an unsubstituted C₁-C₃ alkyl, an unsubstituted C₁-C₂ alkyl, or an unsubstituted methyl. In some forms, R₄ can be an unsubstituted linear or branched C₁-C₅ alkyl, an unsubstituted linear or branched C₁-C₄ alkyl, an unsubstituted linear or branched C₁-C₃ alkyl, an unsubstituted C₁-C₂ alkyl, or an unsubstituted methyl.

For any of Formulae I, Ia, II, III, IV, and XIX, -L₃-A₃, can be independently a substituted or unsubstituted alkyl. In some forms, -L₃-A₃ can be independently a substituted or unsubstituted C₁-C₆ alkyl, a substituted or unsubstituted C₁-C₅ alkyl, a substituted or unsubstituted C₁-C₄ alkyl, a substituted or unsubstituted C₁-C₃ alkyl, a substituted or unsubstituted C₁-C₂ alkyl, or a substituted or unsubstituted methyl. For any of Formulae I, Ia, II, III, IV, and XIX, -L₃-A₃, -L₄-A₄, -L₅-A₅, and/or -L₇-A₇ can be independently

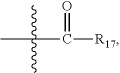

R₁₇ can be a substituted or unsubstituted alkyl, a substituted or unsubstituted aryl or

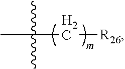

m can be an integer from 0 to 5 and R₂₆ can be a carboxylic acid, an ester, an amino, or an amide. In some forms, R₁₇ can be a substituted or unsubstituted C₁-C₈ alkyl, a substituted or unsubstituted C₁-C₇ alkyl, a substituted or unsubstituted C₁-C₆ alkyl, a substituted or unsubstituted C₁-C₅ alkyl, a substituted or unsubstituted C₁-C₄ alkyl, a substituted or unsubstituted C₁-C₃ alkyl, a substituted or unsubstituted C₁-C₂ alkyl, or a substituted or unsubstituted methyl. In some forms, R₁₇ can be an unsubstituted C₁-C₆ alkyl. In some forms, R₁₇ can be an unsubstituted C₆ cycloalkyl. In some forms, R₁₇ can be an unsubstituted C₁-C₅ alkyl, an unsubstituted C₁-C₄ alkyl, an unsubstituted C₁-C₃ alkyl, an unsubstituted C₁-C₂ alkyl, or an unsubstituted methyl. In some forms, R₁₇ can be an unsubstituted linear or branched C₁-C₅ alkyl, an unsubstituted linear or branched C₁-C₄ alkyl, an unsubstituted linear or branched C₁-C₃ alkyl, an unsubstituted C₁-C₂ alkyl, or an unsubstituted methyl. In some forms, R₁₇ can be a substituted or unsubstituted phenyl. In some forms, R₁₇ can be an unsubstituted phenyl. In some forms, -L₃-A₃ can be

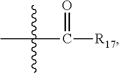

R₁₇ can be an unsubstituted C₁-C₁₀ alkyl or

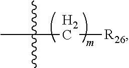

m can be an integer from 0 to 5 and R₂₆ can be a carboxylic acid, an ester, an amino, or an amide. In some embodiments, -L₃-A₃ can be

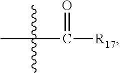

R₁₇ can be an unsubstituted C₁-C₁₀ alkyl, an unsubstituted C₁-C₈ alkyl, an unsubstituted C₁-C₆ alkyl, or an unsubstituted C₁-C₅ alkyl. In some forms, R₂₆ can be an ester.

In some forms, -L₁-A₁ is different from -L₂-A₂, and wherein the compound has the structure of

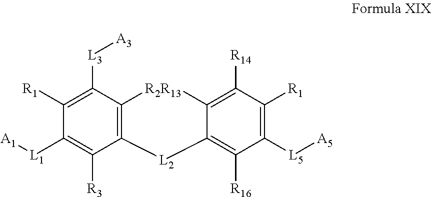

wherein: (a) L₁ and L₂ are independently a substituted or unsubstituted alkylene, or —C(O)—; (b) A₁ is a substituted or unsubstituted aryl, or an C₁-C₆ alkoxy; (c) R₁—R₃ are a hydroxyl; (d) R₁₃—R₁₆ are independently a hydroxyl, a hydrogen, a substituted or unsubstituted alkyl, or —OR₄, R₄ is a substituted or unsubstituted C₁-C₆ alkyl; (e) -L₃-A₃ is

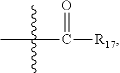

R₁₇ is a substituted or unsubstituted C₁-C₆ alkyl, a substituted or unsubstituted aryl; and (f) -L₅-A₅ is a hydrogen, a substituted or unsubstituted C₁-C₆ alkyl, or

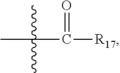

R₁₇ is a substituted or unsubstituted C₁-C₆ alkyl.

In some forms, A₁ is an alkoxy substituted phenyl. In some forms, A₁ is an unsubstituted phenyl. In some forms, -L₃-A₃ is

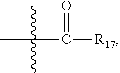

R₁₇ is an unsubstituted phenyl.

In some forms, -L₁-A₁ is different from -L₂-A₂, and wherein the compound has the structure of

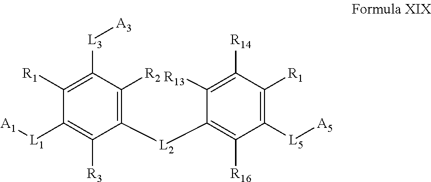

wherein: (a) L₁ and L₂ are independently a substituted or unsubstituted alkylene, or —C(O)—; (b) A₁ is an C₁-C₆ alkoxy; (c) R₁—R₃ are a hydroxyl; (d) R₁₃—R₁₆ are a hydroxyl, a substituted or unsubstituted C₁-C₆ alkyl, or —OR₄, R₄ is a substituted or unsubstituted C₁-C₆ alkyl; (e) -L₃-A₃ is

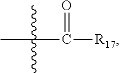

R₁₇ is a substituted or unsubstituted C₁-C₆ alkyl; and (f) -L₅-A₅ is

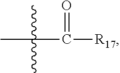

R₁₇ is a substituted or unsubstituted C₁-C₆ alkyl.

For any of Formulae I, Ia, II, III, IV, and XIX, the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl (e.g. benzyl), a carbonyl (e.g. carboxyl), an alkoxy (e.g. methoxy, ethoxy, aryloxy, benzoether, etc.), a halide, a hydroxyl, or a haloalkyl, or a combination thereof.

For any of Formulae I, Ia, II, III, IV, and XIX, the substituents can be independently an unsubstituted alkyl, an unsubstituted alkenyl, an unsubstituted alkynyl, an unsubstituted heterocyclyl, an unsubstituted aryl, an unsubstituted heteroaryl, an unsubstituted polyaryl, an unsubstituted polyheteroaryl, an unsubstituted aralkyl (e.g. benzyl), an alkoxy (e.g. methoxy, ethoxy, aryloxy, benzoether, etc.), a carbonyl (e.g. carboxyl), a halide, a hydroxyl, or a haloalkyl, or a combination thereof.

For any of Formulae I, Ia, II, III, IV, and XIX, the substituents can be an unsubstituted alkyl (e.g. methyl, ethyl, propyl, butyl, pentyl, hexyl, etc.), an alkoxy (e.g. methoxy, ethoxy, etc.), a hydroxyl, or a combination thereof.

For any of Formulae I, Ia, II, III, IV, and XIX, the alkyl can be a linear alkyl, a branched alkyl, or a cyclic alkyl (either monocyclic or polycyclic). The terms “cyclic alkyl” and “cycloalkyl” are used interchangably herein. Exemplary alkyl include a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, or C₁-C₂ alkyl group, a branched C₃-C₉, C₃-C₉, C₃-C₈, C₃-C₇, C₃-C₆, C₃-C₅, or C₃-C₄ alkyl group, or a cyclic C₃-C₉, C₃-C₉, C₃-C₈, C₃-C₇, C₃-C₆, C₃-C₅, or C₃-C₄ alkyl group. The cyclic alkyl can be a monocyclic or polycyclic alkyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄- C₈, C₄-C₇, C₄-C₆, or C₄-C₅ monocyclic or polycyclic alkyl group.

For any of Formulae I, Ia, II, III, IV, and XIX, the alkenyl can be a linear alkenyl, a branched alkenyl, or a cyclic alkenyl (either monocyclic or polycyclic). The terms “cyclic alkenyl” and “cycloalkenyl” are used interchangably herein. Exemplary alkenyl include a linear C₁-C₃₀ alkenyl, a branched C₄-C₃₀ alkenyl, a cyclic C₃-C₃₀ alkenyl, a linear C₁-C₂₀ alkenyl, a branched C₄-C₂₀ alkenyl, a cyclic C₃-C₂₀ alkenyl, a linear C₁-C₁₀ alkenyl, a branched C₄-C₁₀ alkenyl, a cyclic C₃-C₁₀ alkenyl, a linear C₁-C₆ alkenyl, a branched C₄-C₆ alkenyl, a cyclic C₃-C₆ alkenyl, a linear C₁-C₄ alkenyl, cyclic C₃-C₄ alkenyl, such as a linear C₁-C₁₀, C₁-C₉, C₁-C₈, C₁-C₇, C₁-C₆, C₁-C₅, C₁-C₄, C₁-C₃, C₁-C₂ alkenyl group, a branched C₃-C₉, C₃-C₉, C₃-C₈, C₃-C₇, C₃-C₆, C₃-C₅, C₃-C₄ alkenyl group, or a cyclic C₃-C₉, C₃-C₉, C₃-C₈, C₃-C₇, C₃-C₆, C₃-C₅, C₃-C₄ alkenyl group. The cyclic alkenyl can be a monocyclic or polycyclic alkenyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocylcic or polycyclic alkenyl group.

For any of Formulae I, Ia, II, III, IV, and XIX, the alkynyl can be a linear alkynyl, a branched alkynyl, or a cyclic alkynyl (either monocyclic or polycyclic). The terms “cyclic alkynyl” and “cycloalkynyl” are used interchangably herein. Exemplary alkynyl include a linear C₁-C₃₀ alkynyl, a branched C₄-C₃₀ alkynyl, a cyclic C₃-C₃₀ alkynyl, a linear C₁-C₂₀ alkynyl, a branched C₄-C₂₀ alkynyl, a cyclic C₃-C₂₀ alkynyl, a linear C₁-C₁₀ alkynyl, a branched C₄-C₁₀ alkynyl, a cyclic C₃-C₁₀ alkynyl, a linear C₁-C₆ alkynyl, a branched C₄-C₆ alkynyl, a cyclic C₃-C₆ alkynyl, a linear C₁-C₄ alkynyl, cyclic C₃-C₄ alkynyl, such as a linear C₁-C₁₀, C₁-C₉, C₁-C₈, C₁-C₇, C₁-C₆, C₁-C₅, C₁-C₄, C₁-C₃, C₁-C₂ alkynyl group, a branched C₃-C₉, C₃-C₉, C₃-C₈, C₃-C₇, C₃-C₆, C₃-C₅, C₃-C₄ alkynyl group, or a cyclic C₃-C₉, C₃-C₉, C₃-C₈, C₃-C₇, C₃-C₆, C₃-C₅, C₃-C₄ alkynyl group. The cyclic alkynyl can be a monocyclic or polycyclic alkynyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄- C₆, or C₄-C₅ monocyclic or polycyclic alkynyl group.

It is understood that any of the exemplary alkyl, alkenyl, and alkynyl groups can be heteroalkyl, heteroalkenyl, and heteroalkynyl, respectively. For example, the alkyl can be a linear C₂-C₃₀ heteroalkyl, a branched C₄-C₃₀ heteroalkyl, a cyclic C₃-C₃₀ heteroalkyl (i.e. a monocycloheteroalkyl or polycycloheteroalkyl), a linear C₁-C₂₀ heteroalkyl, a branched C₄-C₂₀ heteroalkyl, a cyclic C₃-C₂₀ heteroalkyl, a linear C₁-C₁₀ heteroalkyl, a branched C₄-C₁₀ heteroalkyl, a cyclic C₃-C₁₀ heteroalkyl, a linear C₁-C₆ heteroalkyl, a branched C₄-C₆ heteroalkyl, a cyclic C₃-C₆ heteroalkyl, a linear C₁-C₄ heteroalkyl, cyclic C₃-C₄ heteroalkyl, such as a linear C₁-C₁₀, C₁-C₉, C₁-C₈, C₁-C₇, C₁-C₆, C₁-C₅, C₁-C₄, C₁-C₃, C₁-C₂ heteroalkyl group, a branched C₃-C₉, C₃-C₉, C₃-C₈, C₃-C₇, C₃-C₆, C₃-C₅, C₃-C₄ heteroalkyl group, or a cyclic C₃-C₉, C₃-C₉, C₃-C₈, C₃-C₇, C₃-C₆, C₃-C₅, C₃-C₄ heteroalkyl group. The cyclic heteroalkyl can be monocyclic or polycyclic, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄- C₇, C₄-C₆, or C₄-C₅ monocyclic or polycyclic heteroalkyl group.

For any of Formulae I, Ia, II, III, IV, and XIX, the alkenyl can be a linear C₂-C₃₀ heteroalkenyl, a branched C₄-C₃₀ heteroalkenyl, a cyclic C₃-C₃₀ heteroalkenyl (i.e. a monocycloheteroalkenyl or polycycloheteroalkenyl), a linear C₁-C₂₀ heteroalkenyl, a branched C₄-C₂₀ heteroalkenyl, a cyclic C₃-C₂₀ heteroalkenyl, a linear C₁-C₁₀ heteroalkenyl, a branched C₄-C₁₀ heteroalkenyl, a cyclic C₃-C₁₀ heteroalkenyl, a linear C₁-C₆ heteroalkenyl, a branched C₄-C₆ heteroalkenyl, a cyclic C₃-C₆ heteroalkenyl, a linear C₁-C₄ heteroalkenyl, cyclic C₃-C₄ heteroalkenyl, such as a linear C₁-C₁₀, C₁-C₉, C₁-C₈, C₁-C₇, C₁-C₆, C₁-C₅, C₁-C₄, C₁-C₃, C₁-C₂ heteroalkenyl group, a branched C₃-C₉, C₃-C₉, C₃-C₈, C₃-C₇, C₃-C₆, C₃-C₅, C₃-C₄ heteroalkenyl group, or a cyclic C₃-C₉, C₃-C₉, C₃-C₈, C₃-C₇, C₃-C₆, C₃-C₅, C₃-C₄ heteroalkenyl group. The cyclic heteroalkenyl can be monocyclic or polycyclic, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic heteroalkenyl group.

For any of Formulae I, Ia, II, III, IV, and XIX, the alkynyl can be a linear C₂-C₃₀ heteroalkynyl, a branched C₄-C₃₀ heteroalkynyl, a cyclic C₃-C₃₀ heteroalkynyl (i.e. a monocycloheteroalkynyl or polycycloheteroalkynyl), a linear C₁-C₂₀ heteroalkynyl, a branched C₄-C₂₀ heteroalkynyl, a cyclic C₃-C₂₀ heteroalkynyl, a linear C₁-C₁₀ heteroalkynyl, a branched C₄-C₁₀ heteroalkynyl, a cyclic C₃-C₁₀ heteroalkynyl, a linear C₁-C₆ heteroalkynyl, a branched C₄-C₆ heteroalkynyl, a cyclic C₃-C₆ heteroalkynyl, a linear C₁-C₄ heteroalkynyl, cyclic C₃-C₄ heteroalkynyl, such as a linear C₁-C₁₀, C₁-C₉, C₁-C₈, C₁-C₇, C₁-C₆, C₁-C₅, C₁-C₄, C₁-C₃, C₁-C₂ heteroalkynyl group, a branched C₃-C₉, C₃-C₉, C₃-C₈, C₃-C₇, C₃-C₆, C₃-C₅, C₃-C₄ heteroalkynyl group, or a cyclic C₃-C₉, C₃-C₉, C₃-C₈, C₃-C₇, C₃-C₆, C₃-C₅, C₃-C₄ heteroalkynyl group. The cyclic heteroalkynyl can be monocyclic or polycyclic, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic heteroalkynyl group.

For any of Formulae I, Ia, II, III, IV, and XIX, the aryl group can be a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁₁ aryl, or a C₆-C₉ aryl. It is understood that the aryl can be a heteroaryl, such as a C₅-C₃₀ heteroaryl, a C₅-C₂₀ heteroaryl, a C₅-C₁₂ heteroaryl, a C₅-C₁₁ heteroaryl, a C₅-C₉ heteroaryl, a C₆-C₃₀ heteroaryl, a C₆-C₂₀ heteroaryl, a C₆-C₁₂ heteroaryl, a C₆-C₁₁ heteroaryl, or a C₆-C₉ heteroaryl. For any of Formulae I, Ia, II, III, and IV, the polyaryl group can be a C₁₀-C₃₀ polyaryl, a C₁₀-C₂₀ polyaryl, a C₁₀-C₁₂ polyaryl, a C₁₀-C₁₁ polyaryl, or a C₁₂-C₂₀ polyaryl. It is understood that the aryl can be a polyheteroaryl, such as a C₁₀-C₃₀ polyheteroaryl, a C₁₀-C₂₀ polyheteroaryl, a C₁₀-C₁₂ polyheteroaryl, a C₁₀-C₁₁ polyheteroaryl, or a C₁₂-C₂₀ polyheteroaryl.

In some forms, the compound is not

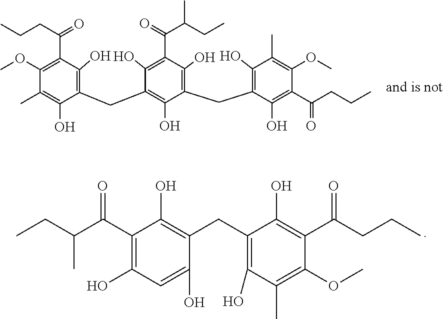

In some forms, the compound is not

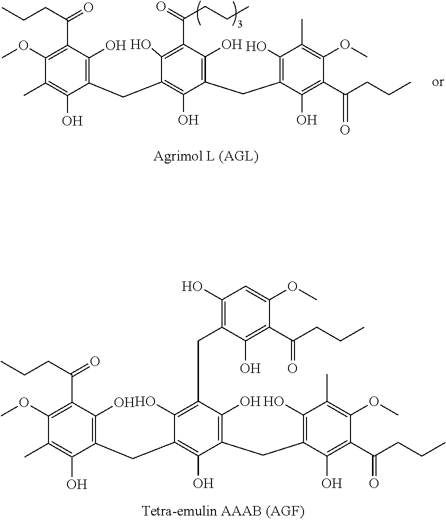

In some forms, the compound is:

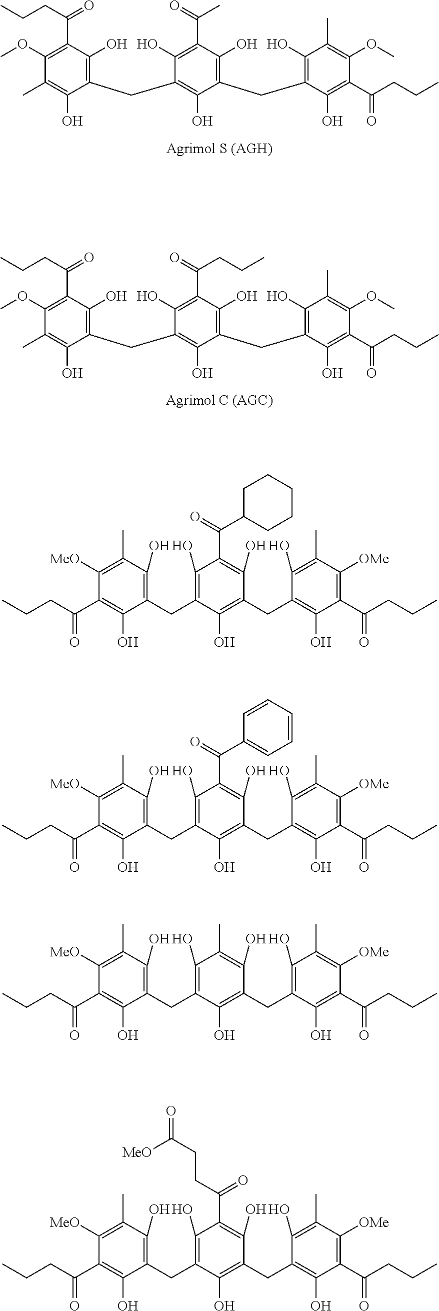

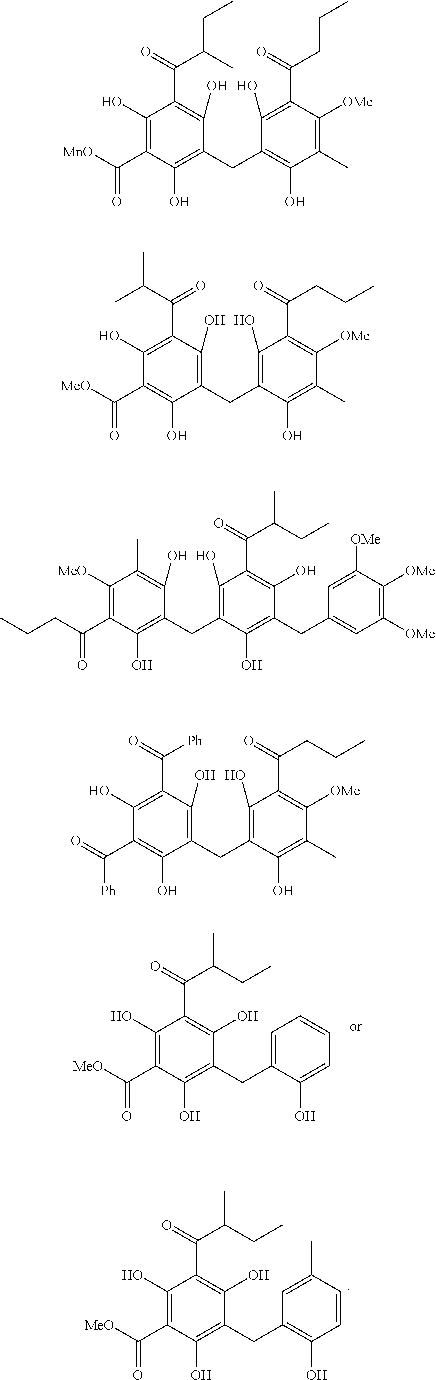

The compounds may be neutral or may be one or more pharmaceutically acceptable salts, crystalline forms, non-crystalline forms, hydrates, or solvates, or a combination thereof. References to the compounds may refer to the neutral molecule, and/or those additional forms thereof collectively and individually from the context. Pharmaceutically acceptable salts of the compounds include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

### B. Pharmaceutical Formulations

Pharmaceutical formulations that contain one or more the compounds disclosed herein, in a form suitable for administration to a mammal, are disclosed. Typically, the compound(s) in the pharmaceutical formulation is present in an amount effective to treat a cancer, reduce a cancer, or treat or ameliorate one or more symptoms associated with a cancer in a subject. In some forms, the compound(s) in the pharmaceutical formulation is present in an amount effective to induce mitochondria dysfunction and/or to inhibit ER to Golgi trafficking in cancer cells in the subject. In some forms, the compound(s) in the pharmaceutical formulation is present in an amount effective to induce apoptosis of cancer cells in the subject. In some forms, the compound(s) in the pharmaceutical formulation is present in an amount effective to inhibit the growth of a tumor by at least 40%, at least 50%, or at least 55% in the subject compared to the same tumor in a control subject.

The pharmaceutical formulation containing the compound(s) may also include one more more pharmaceutically acceptable carriers and/or one or more pharmaceutically acceptable excipients. For example, the pharmaceutical formulation may be in the form of a liquid, such as a solution or a suspension, and contain one or more the disclosed compounds in an aqueous medium and, optionally, one or more suitable excipients for the liquid formulation. Optionally, the pharmaceutical formulation is in a solid form, and contains one or more the disclosed compounds and one or more suitable excipients for a solid formulation.

### 1. Carriers and Excipients

The pharmaceutical formulation can contain one or more pharmaceutically acceptable carriers and/or one or more pharmaceutically acceptable excipients. Suitable pharmaceutically acceptable carriers and excipients are generally recognized as safe (GRAS), and may be administered to an individual without causing undesirable biological side effects or unwanted interactions.

Representative carriers and excipients that can be used in the pharmaceutical formulations include solvents (including buffers), diluents, pH modifying agents, preservatives, antioxidants, suspending agents, wetting agents, viscosity modifiers, tonicity agents, and stabilizing agents, and a combination thereof.

In some forms, the compounds can be dissolved or suspended in a suitable carrier to form a liquid pharmaceutical formulation, such as sterile saline, phosphate buffered saline (PBS), balanced salt solution (BSS), viscous gel, or other pharmaceutically acceptable carriers for administration. The pharmaceutical formulation may also be a sterile solution, suspension, or emulsion in a nontoxic, parenterally acceptable diluent or solvent.

Excipients can be added to a liquid or solid pharmaceutical formulation to assist in sterility, stability (e.g. shelf-life), integration, and to adjust and/or maintain pH or isotonicity of the compounds in the pharmaceutical formulation, such as diluents, pH modifying agents, preservatives, antioxidants, suspending agents, wetting agents, viscosity modifiers, tonicity agents, and stabilizing agents, and a combination thereof.

### 2. Form

The pharmaceutical formulation containing one or more the disclosed compounds can be in a liquid form or a solid form, as a liquid formulation or a solid formulation for oral administration or parenteral administration (e.g. intramuscular administration, intravenous administration, intraperitoneal administration, and subcutaneous administration) to a subject.

a. Oral Formulations

In some forms, the pharmaceutical formulation containing one or more the disclosed compounds can be in a form suitable for oral administration to a subject, such as a mammal (i.e. an oral formulation). Oral administration may involve swallowing, so that the compound(s) enter the gastrointestinal tract, or buccal or sublingual administration may be employed by which the compound(s) enter(s) the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays, and liquid formulations.

Liquid formulations for oral administration include suspensions, solutions, syrups, and elixirs. Such oral formulations may be employed as fillers in soft or hard capsules and can contain one or more suitable carriers and/or excipients, for example, water, ethanol, polyethylene glycol, propylene glycol, chitosan polymers and chitosan derivatives (e.g. N-trimethylene chloride chitosan, chitosan esters, chitosan modified with hydrophilic groups, such as amino groups, carboxyl groups, sulfate groups, etc.), methylcellulose, a suitable oil, one or more emulsifying agents, and/or suspending agents. Liquid formulations for oral administration may also be prepared by the reconstitution of a solid, for example, from a sachet.

Optionally, the compound(s) is/are included in a fast-dissolving and/or fast-disintegrating dosage form.

For tablet or capsule dosage forms, in addition to the compound(s) described herein, tablets generally contain disintegrants, binders, diluents, surface active agents, lubricants, glidants, antioxidants, colourants, flavouring agents, preservatives, or taste masking agents, or a combination thereof.

Examples of suitable disintegrants for forming a table or capsule dosage form containing the compound(s) include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant can have a concentration in a range from about 1 wt % to about 25 wt %, from about 5 wt % to about 20 wt % of the tablet or capsule dosage form containing the compound(s).

Binders are generally used to impart cohesive qualities to a tablet formulation. Suitable binders for forming a tablet or capsule formulation containing the compound(s) include, but are not limited to, microcrystalline cellulose, gelatin, sugars, polyethylene glycol, natural and synthetic gums, polyvinylpyrrolidone, pregelatinised starch, chitosan polymers and chitosan derivatives (e.g. N-trimethylene chloride chitosan, chitosan esters, chitosan modified with hydrophilic groups, such as amino groups, carboxyl groups, sulfate groups, etc.), hydroxypropyl cellulose, and hydroxypropyl methylcellulose.

Suitable diluents for forming a table or capsule formulation containing the compound(s) include, but are not limited to, lactose (as, for example, the monohydrate, spray-dried monohydrate or anhydrous form), chitosan polymers and chitosan derivatives (e.g. N-trimethylene chloride chitosan, chitosan esters, chitosan modified with hydrophilic groups, such as amino groups, carboxyl groups, sulfate groups, etc.), N-sulfonated derivatives of chitosan, quaternarized derivatives of chitosan, carbosyalkylated chitosan, microcrystalline chitosan, mannitol, xylitol, dextrose, sucrose, sorbitol, microcrystalline cellulose, starch and dibasic calcium phosphate dihydrate.

Tablet or capsule formulations containing the compound(s) may also contain surface active agents, such as sodium lauryl sulfate and polysorbate 80, and glidants such as silicon dioxide and talc. When present, surface active agents can have a concentration in a range from about 0.2 wt % to 5 wt % of the tablet or capsule formulation.

Tablet or capsule formulations containing the compound(s) also can contain lubricants, such as magnesium stearate, calcium stearate, zinc stearate, sodium stearyl fumarate, and mixtures of magnesium stearate with sodium lauryl sulphate. Lubricants can have a concentration in a range from about 0.25 wt % to 10 wt %, from about 0.5 wt % to about 3 wt % of the tablet or capsule formulation.

Other possible excipients included in a tablet or capsule formulation containing the compound(s) include glidants (e.g. Talc or colloidal anhydrous silica at about 0.1 wt % to about 3 wt % of the table or capsule formulation), antioxidants, colourants, flavouring agents, preservatives and taste-masking agents. When present, glidants can have a concentration in a range from about 0.2 wt % to 1 wt % of the tablet or capsule formulation.

An exemplary tablet formulation contains up to about 80 wt % of the compound(s) described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet or capsule blends, including the compound(s) and one or more suitable excipients, may be compressed directly or by roller to form tablets. Tablet or capsule blends or portions of the blends may alternatively be wet-, dry-, or melt-granulated, melt congealed, or extruded before tableting. The final table or capsule formulation may contain one or more layers and may be coated or uncoated; it may even be encapsulated in a particle, such as a polymeric particle or a liposomal particle.

Solid formulations containing the compound(s) for oral administration may be formulated to be immediate and/or modified release. Modified release formulations include delayed, sustained, pulsed, controlled, targeted and programmed release formulations.

b. Parenteral Formulations

In some forms, the pharmaceutical formulation containing one or more the disclosed compounds can be in a form suitable for administration directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

For example, the pharmaceutical formulation containing one or more the compounds is in a form suitable for intramuscular administration, intravenous administration, intraperitoneal administration, or subcutaneous administration, or a combination thereof.

Parenteral formulations containing the compound(s) described herein are typically aqueous solutions which can contain excipients such as salts, carbohydrates and buffering agents (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4), but, for some applications, they may be more suitably formulated as a sterile aqueous solution or as a dried form to be used in conjunction with a suitable vehicle such as sterile, pyrogen-free water.

The liquid formulations containing the compound(s) for parenteral administration may be a solution, a suspension, or an emulsion.

The liquid pharmaceutically acceptable carrier forming the parenteral formulation containing the compound(s) can include one or more physiologically compatible buffers, such as a phosphate buffers. One skilled in the art can readily determine a suitable saline content and pH for an aqueous carrier for administration (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4).

Liquid formulations containing the compound(s) for parenteral administration may include one or more suspending agents, such as cellulose derivatives, sodium alginate, polyvinylpyrrolidone, gum tragacanth, or lecithin. The liquid formulations may also include one or more preservatives, such as ethyl or n-propylp-hydroxybenzoate.

In some forms, the liquid formulation containing the compound(s) contains one or more solvents that are low toxicity organic (i.e., nonaqueous) class 3 residual solvents, such as ethanol, acetone, ethyl acetate, tetrahydofuran, ethyl ether, and propanol, and a combination thereof. Any such solvents included in the liquid formulation should not detrimentally react with the compound(s) and any additional active agents when present in the liquid formulation. Solvents such as freon, alcohol, glycol, polyglycol, or fatty acid, can also be included in the liquid formulation containing the compound(s) as desired to increase the volatility of the solution or suspension.

Liquid formulations containing the compound(s) for parenteral administration may also contain minor amounts of polymers, surfactants, or other pharmaceutically acceptable excipients known to those in the art. In this context, “minor amounts” means an amount that is sufficiently small to avoid adversely affecting uptake of the compounds by the targeted cells, such as pituitary gonadotrophs.

The preparation of parenteral formulations containing the compound(s) is typically under sterile conditions, for example, by lyophilisation, which can be accomplished using standard pharmaceutical techniques known to those skilled in the art.

Formulations for parenteral administration containing the compound(s) may be formulated to provide immediate and/or modified release of the active agent. Modified release formulations include delayed, sustained, pulsed, controlled, targeted and programmed release formulations.

c. Pulmonary and Mucosal Formulations

In some forms, the pharmaceutical formulation containing one or more the disclosed compounds can be in a form suitable for pulmonary or mucosal administration. The administration can include delivery of the composition to the lungs, nasal, oral (sublingual, buccal), vaginal, or rectal mucosa.

For example, the compounds can be administered intranasally or by oral inhalation, such as in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (such as an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may comprise a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebuliser contains a solution or suspension of one or more of the compounds including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, a drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compounds described herein, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation containing the compound(s) for use in an atomizer using electrohydrodynamics to produce a fine mist may contain from 1 μg to 20 mg of one or more of the compounds per actuation and the actuation volume may vary from 1 μl to 100 μl. A typical formulation may contain one or more of the compounds described herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the compounds are typically arranged to administer a metered dose or “puff.” The overall daily dose will be administered in a single dose or, more usually, as divided doses throughout the day.

In some forms, the compounds can be formulated for pulmonary delivery, such as intranasal administration or oral inhalation. Carriers for pulmonary formulations can be divided into those for dry powder formulations and for administration as solutions. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e. nonaqueous) class 3 residual solvents, such as ethanol, acetone, ethyl acetate, tetrahydrofuran, ethyl ether, and propanol may be used for the formulations containing the compound(s). The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In some forms, the pharmaceutical formulations containing the compound(s) may contain minor amounts of polymers, surfactants, or other excipients well known to those of the art. In this context, “minor amounts” means no excipients are present that might affect or mediate uptake of the compounds by cells and that the excipients that are present in amount that do not adversely affect uptake of compounds by cells.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Non-aqueous suspensions of lipids can also be prepared in absolute ethanol using a reusable PARI LC Jet+ nebulizer (PARI Respiratory Equipment, Monterey, CA).

d. Topical Formulations

The compounds can be administered directly to the external surface of the skin or the mucous membranes (including the surface membranes of the nose, lungs and mouth), such that the compounds can cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Formulations containing the compound(s) for topical administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the compounds. Examples of such formulations include drug-coated stents and poly(dl-lactic-coglycolic)acid (PGLA) microspheres.

### 3. Additional Active Agent(s)

In some forms, the pharmaceutical formulation can include one or more additional active agents, such as one or more additional anticancer agents. Anticancer agents that can be included in the pharmaceutical compositions or formulations are known, for example, see the National Cancer Institute database, “A to Z List of Cancer Drugs,” website cancer.gov/about-cancer/treatment/drugs.

Exemplary anticancer drugs that can be included in the pharmaceutical formulation containing the compound(s) include, but are not limited to, olaparib, abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

### 4. Dosages/wt % Concentration

In some forms, the pharmaceutical formulation contains an effective amount of the comopund(s) for treating a cancer, reducing a cancer, or treating or ameliorating one or more symptoms associated with a cancer in a subject.

In some forms, the comopund(s) is present in the pharmaceutical formulation in an effective amount to induce mitochondria dysfunction and/or to inhibit ER to Golgi trafficking in cancer cells in the subject.

In some forms, the pharmaceutical formulation contains the compound(s) in an amount that is effective to induce mitochondria dysfunction as shown by mitochondria fission, decreased ATP production, and/or decreased membrane potential depolarization, compared to cancer cells in the subject before treatment with the compound(s). Mitochondira fission can be determined by a morphology change of mitochondira to punctuated structure measured by imaging; a decreased expression of fission markers, such as Muff and/or DRP1, by at least 5%; a translocation of DRP1 from cytoplasma into mitochondria; and/or OPA1 fragmentation, compared to mitochondria of the cancer cells before treatment with the compound(s).

In some forms, the pharmaceutical formulation contains the compound(s) in an amount that is effective to inhibit ER to Golgi trafficking in cancer cells in the subject as shown by a redistribution and/or disruption of Golgi measured by imaging and/or ER stress, compared to cancer cells in the subject before treatment with the compound(s). ER stress can be determined by a disrupted morphology of the ER as measured by imaging; an increased expression of ER stress markers, such as glucose-regulated proten 78 (GRP78) and C/-EBP homologous protein (CHOP); and/or the presence of phosphorylated eIF2α.

In some forms, the pharmaceutical formulation contains the compound(s) in an amount that is effective to induce mitochondria dysfunction and to inhibit ER to Golgi trafficking in cancer cells in the subject as shown by the parameters described above, compared to cancer cells in the subject before treatment with the compound(s).

In some forms, the compound(s) is present in the pharmaceutical formulation in an effective amount to induce apoptosis of cancer cells in the subject. Whether apoptosis of cancer cells is induced may be be identified by a change of at least 5% in relevant biomarker or gene expression profile in a biological sample of the subject, compared to the biomarker or gene expression profile in the biological sample of the subject before treatment with the compound(s). Exemplary biomarkers for showing apoptosis of cancer cells includes, but are not limited to, HER2 for breast cancer; PSA for prostate cancer; carcinoembryonic antigen (CEA), squamous cell carcinoma antigen (SCC), neuron-specific enolase (NSE), cytokeratin 19 fragment (CYFRA), and pro-gastrin-releasing peptide (proGRP) for lung cancer, or others. Alternatively or additionally, whether apoptosis of cancer cells is induced may be be identified by chromatin condensation and fragmentation; and/or cleavages of PARP, caspase-9, and/or caspase-3. In some forms, the pharmaceutical formulation contains the compound(s) in an amount that is effective to induce apoptosis of cancer cells as shown by chromatin condensation and fragmentation; cleavages of PARP, caspase-9, and/or caspase-3; and/or changes in the levels of proteins involved in apoptotic signaling using Western blotting.

In some forms, the compound(s) is present in the pharmaceutical formulation in an effective amount to inhibit the growth of a tumor by at least 40%, at least 50%, or at least 55% in the subject compared to the same tumor in a control subject. Inhibition of tumor can be determined by known methods, such as by measuring the size of an isolated solid tumor or measuring the tumor size in vivo using imaging. In some forms, the pharmaceutical formulation contains the compound(s) in an amount that is effective to inhibit the growth of a tumor by at least 40%, at least 50%, or at least 55% in the subject, compared to the same tumor in a control subject by the end of a monitoring time period, such as by the end of 21 days, 28 days, 31 days, 2 months, 3 months, 6 months, or a year, optionally without any toxicity as shown by a stable body weight of the subject having the tumor. A stable body weight refers to a body weight change of less than about 10% during and by the end of the monitoring time period.

The total concentration of the compound(s) in the pharmaceutical formulation can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %. The term “total concentration of the compound(s) in the pharmaceutical formulation” refers to the sum of the weight of all compound(s) relative to the weight of the formulation.

In some forms, the total concentration of the compound(s) in the pharmaceutical formulation that is effective to treat a cancer, reduce a cancer, or treat or ameliorate one or more symptoms associated with a cancer in a subject, can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %. In some forms, the total concentration of the compound(s) in the pharmaceutical formulation that is effective to induce mitochondria dysfunction and/or to inhibit ER to Golgi trafficking in cancer cells in the subject can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the total concentration of the compound(s) in the pharmaceutical formulation that is effective to induce apoptosis of cancer cells in the subject can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the total concentration of the compound(s) in the pharmaceutical formulation that is effective to inhibit the growth of a tumor by at least 40%, at least 50%, or at least 55% in the subject compared to the same tumor in a control subject, by the end of a monitoring period, can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the pharmaceutical formulation containing the compound(s) can be provided in a unit dosage form. The dosage of the compounds in the pharmaceutical formulation in the unit dosage form can be in a range from about 0.002 mg to about 1 mg, in a range from about 0.006 mg to about 0.6 mg, in a range from about 0.01 mg to about 0.4 mg, in a range from about 0.02 mg to about 0.3 mg, or in a range from about 0.01 mg to about 0.2 mg.

### III. Methods for Synthesis of AGB and Derivatives Thereof

Methods for synthesizing AGB and derivatives thereof with a high overall yield (i.e. an overall yield ≥7%) have been developed. The methods described herein for total synthesis of AGB and derivatives thereof use an acylation step and an methylation step to obtain an intermediate and employ an Eschenmoser methenylation reaction to directly form AGB or a derivative thereof. The overall yield of the AGB or derivative thereof can be at least 7%, at least 10%, at least 15%, at least 20%, at least 25%, or at least 30% (which is calculated from the starting material using the cumulative yield from each step). The methods described herein can overcome the limitation of amount scarcity associated with previous methods (L₁, et al., Acta Chimica Sinica, 36(1):43-48 (1978); Pei, et al., Acta Pharmaceutica Sinica 24 (1989) 431-437; and CN108264454 by Huang, et al.) and provide a feasible route for the total synthesis of AGB and its derivatives. For example, compared to the syntheses described in L₁, et al., Acta Chimica Sinica, 36(1):43-48 (1978) and Pei, et al., Acta Pharmaceutica Sinica 24 (1989) 431-437, the synthesis of AGB and derivatives thereof described herein has a higher yield, involves less steps, provides more controllable synthetic conditions, and has a higher atomic efficiency. For example, compared to CN108264454 by Huang, et al., the synthesis of AGB and derivatives thereof described herein uses simple and readily available starting materials, does not require any catalysts in the last step and thereby is more environment-friendly, and provides a high overall yield.

The method generally includes: (i) heating a reaction mixture at a suitable temperature for a period of time sufficient to form a product. The reaction mixture can contain a first reactant, a second reactant, a third reactant, and a solvent. The structures of the reactants are described in detail below. The product contains AGB or a derivative of AGB.

In some forms, the method includes (i-a) converting a pre-reactant to the second and third reactants and/or (i-b) converting a starting material to the pre-reatant. In some forms, the starting material can be first converted to an intermediate and then the intermediate can be converted to the pre-reactant. The structures of the starting materials, intermediates, pre-reactants, and reactants are described in detail below.

In some forms, the method also includes mixing the first reactant, the second reactant, the third reactant, and the solvent to form the reaction mixture prior to step (i); stirring the reaction mixture prior to and/or during step (i); and/or purifying the product, optionally by flash column chromatography, subsequent to step (i).

More specific methods for synthesizing AGB and exemplary AGB derivatives are described in the Examples below.

### A. Heating a Reaction Mixture at a Temperature for a Period of Time Sufficient to Form a Product Containing AGB or Derivatives Thereof

Generally, a reaction mixture containing the reactants in a suitable solvent is heated at a suitable temperature for a period of time sufficient to form a product containing AGB or a derivative thereof. Typically, the reaction conditions for performing the reaction are mild. For example, the temperature for performing the reaction can be up to 130° C., up to 120° C., in a range from about 100° C. to about 130° C., from about 100° C. to about 125° C., from about 100′C to about 120° C., at 1 atm, such as about 110° C., at 1 atm; the time period for performing the reaction can be up to 5 hours, up to 4 hours, up to 2 hours, or up to 1 hour, in a range from about 30 minutes to about 5 hours, from about 30 minutes to about 4 hours, from about 30 mins to about 3.5 hours, from about 30 mins to about 3 hours, from about 30 mins to about 2.5 hours, from about 1 hour to about 5 hours, from about 1 hour to about 4 hours, from about 1 hour to 3 hours, or from about 1 hour to about 2 hours; and optionally the reaction can be performed under reflux.

The reaction mixture can be formed by dissolving the reactants in the solvent prior to reaction. Suitable solvents for forming the reaction mixture can dissolve each of the reactants. For example, each reactant has a solubility of at least 0.01 M in the solvent. Examples of solvents suitable for forming the reaction mixture include, but are not limited to dichloromethane, dimethyl sulfate, dimethyl sulfoxide, dimethyl formamide, butyryl chloride, dichloroethane, nitrobenzene, dioxane, 2-methylbutyryl chloride, ethyl acetate, ethyl lactate, acetone, 1-butanol, 1-propanol, 2-propanol, ethanol, isopropyl acetate, methanol, methyl ethyl ketone, t-butanol, tetrahydrofuran, 2-methyl tetrahydrofuran, acetonitrile, or toluene, or a combination thereof, or a mixture of water and an organic solvent as described above.

The reaction mixture contains a first reactant, a second reactant, and a third reactant. The first reactant can have the structure of Formula V:

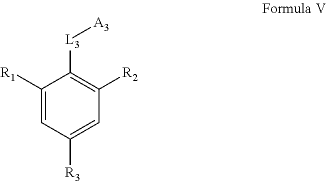

the second reactant can have the structure of Formula VI:

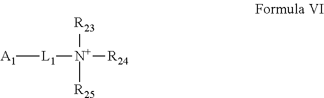

the third reactant can have the structure of Formula VII:

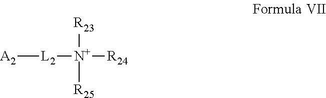

wherein: (a) L₁ and L₂ can be independently a bond (single, double, or triple), a substituted or unsubstituted alkyl, a substituted or unsubstituted alkenyl, a carbonyl, or an alkoxy; (b) L₃ can be a bond (single, double, or triple), a substituted or unsubstituted alkyl, or a carbonyl; (c) A₁ and A₂ can be independently a hydrogen, a substituted or unsubstituted alkyl, a substituted or unsubstituted alkenyl, a substituted or unsubstituted aryl, a carbonyl, or an alkoxy, and at least one of A₁ and A₂ is a substituted or unsubstituted aryl; (d) A₃ can be a hydrogen, a substituted or unsubstituted alkyl, a substituted or unsubstituted aryl, a carbonyl, or an alkoxy; (e) R₁—R₃ can be independently a hydrogen, a hydroxyl, a substituted or unsubstituted alkyl, a carbonyl, an alkoxy, a thiol, or an amino; (f) R₂₃—R₂₅ can be independently a hydrogen or a substituted or unsubstituted alkyl; and (g) the substituents can be as defined above for Formula I.

The second reactant can be the same as or different from the third reactant. In some forms, the second reactant can be the same as the third reactant. In some forms, the second reactant can be different from the third reactant.

In some forms, -L₃-A₃ can be

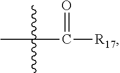

R₁₇ can be an an unsubstituted C₁-C₁₀ alkyl or

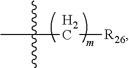

m can be an integer from 0 to 5 and R₂₆ can be a carboxylic acid, an ester, an amino, or an amide.

In some forms, A₁ can be

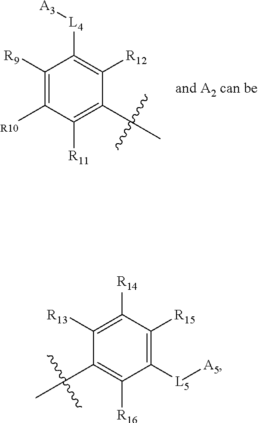

wherein: (a) L₄ and L₅ can be independently a bond (single, double, or triple), a substituted or unsubstituted alkyl, or a carbonyl; (b) A₄ and A₅ can be independently a hydrogen, a substituted or unsubstituted alkyl, a substituted or unsubstituted aryl, a carbonyl, or an alkoxy; (c) R₉—R₁₆ can be independently a hydrogen, a hydroxyl, a substituted or unsubstituted alkyl, a carbonyl, an alkoxy, a thiol, or an amino; and (d) the substituents can be as defined above for Formula I.

In some forms, the second reactant can be the same as the third reactant. When the second reactant is the same as the third reactant, the reaction mixture contains two reactants: a first reactant and a second reactant. In these forms, the second reactant can have the structure of Formula VIII:

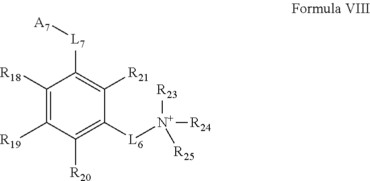

wherein: (a) L₆ can be a bond (single, double, or triple), a substituted or unsubstituted alkyl, a substituted or unsubstituted alkenyl, a carbonyl, or an alkoxy; (b) L₇ can be a bond (single, double, or triple), a substituted or unsubstituted alkyl, or a carbonyl; (c) A₇ can be a hydrogen, a substituted or unsubstituted alkyl, a substituted or unsubstituted aryl, a carbonyl, or an alkoxy; (d) R₁₈—R₂₁ can be independently a hydrogen, a hydroxyl, a substituted or unsubstituted alkyl, a carbonyl, an alkoxy, a thiol, or an amino; (e) R₂₃—R₂₅ can be independently a hydrogen or a substituted or unsubstituted alkyl; and (f) the substituents can be as defined above for Formula I.

In some forms, the method can further include (i-a) converting a pre-reactant having the structure of Formula VIII′ to the reactant having the structure of Formula VIII:

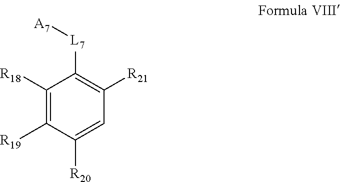

wherein: (a) L₇ can be a bond (single, double, or triple), a substituted or unsubstituted alkyl, or a carbonyl; (b) A₇ can be a hydrogen, a substituted or unsubstituted alkyl, a substituted or unsubstituted aryl, a carbonyl, or an alkoxy; (c) R₁₈—R₂₁ can be independently a hydrogen, a hydroxyl, a substituted or unsubstituted alkyl, a carbonyl, an alkoxy, a thiol, or an amino; and (d) the substituents can be as defined above for Formula I.

In some forms, R₁₉ can be a substituted or unsubstituted alkyl, such as an unsubstituted methyl. In some forms, R₁₈ can be —OR₂₂, each R₂₂ can be hydrogen or a substituted or unsubstituted alkyl. In some forms, R₂₀ and R₂₁ can be hydroxyl. In some forms, R₁₉ can be a substituted or unsubstituted alkyl, such as an unsubstituted methyl; R₁₈ can be —OR₂₂, each R₂₂ can be hydrogen or a substituted or unsubstituted alkyl; and R₂₀ and R₂₁ can be hydroxyl. In some forms, -L₇-A₇ can be

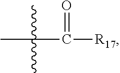

R₁₇ can be an unsubstituted C₁-C₁a alkyl, an unsubstituted C₁-C₈ alkyl, an unsubstituted C₁-C₆ alkyl, an unsubstituted C₁-C₄ alkyl, an unsubstituted C₁-C₃ alkyl, or

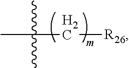

m can be an integer from 0 to 5 and R₂₆ can be a carboxylic acid, an ester, an amino, or an amide. In some forms, -L₇-A₇ can be

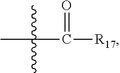

R₁₇ can be an unsubstituted C₁-C₁a alkyl, an unsubstituted C₁-C₈ alkyl, an unsubstituted C₁-C₆ alkyl, an unsubstituted C₁-C₄ alkyl, or an unsubstituted C₁-C₃ alkyl.

For any of Formulae V-VIII and VIII′, the alkyl, alkenyl, alkynyl, aryl, and substituents can be any of those described above for Formula I, Ia, and II-IV.

In some forms, the reaction mixture contains a first reactant having the structure of Formula XII:

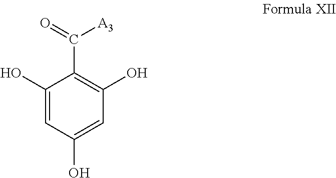

and a second reactant having the structure of Formula XIII:

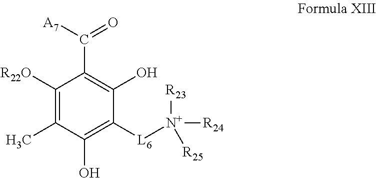

wherein: (a) A₃ and A₇ can be independently a hydrogen, a substituted or unsubstituted alkyl, a substituted or unsubstituted aryl, a carbonyl, or an alkoxy; (b) L₆ can be a bond (single, double, or triple), a substituted or unsubstituted alkyl, a substituted or unsubstituted alkenyl, a carbonyl, or an alkoxy; (c) R₂₂ can be a hydrogen or a substituted or unsubstituted alkyl; (d) R₂₃—R₂₅ can be independently a hydrogen or a substituted or unsubstituted alkyl; and (e) the substituents can be as defined above for Formula I. In some forms, A₃ and A₇ can be independently a substituted or unsubstituted alkyl, such as an unsubstituted alkyl, e.g. an unsubstituted C₁-C₁₀ alkyl, an unsubstituted C₁-C₅ alkyl, an unsubstituted C₁-C₇ alkyl, an unsubstituted C₁-C₆ alkyl, an unsubstituted C₁-C₅ alkyl, an unsubstituted C₁-C₄ alkyl, or an unsubstituted C₁-C₃ alkyl.

The first reactant having the structure of Formula XII can be synthesized by coupling phloroglucinol with a corresponding acyl halide having the structure of

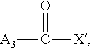

where A₃ can be as defined above for Formula XII and X′ is a halogen atom, such as Cl, Br, I, or F, e.g. Cl.

More specific conditions and reagents for the reaction in step (i) are described in the Examples below.

### B. Converting a Starting Material to a Pre-Reactant

In some forms, the method can include (i-b) converting a suitable starting material to the pre-reactant having the structure of Formula VIII′ above. The conversion from the starting material to the pre-reactant of Formula VIII′ can be achieved via different synthetic routes; each synthetic rout can include a single step, two steps, three steps, four steps, etc. In some forms, the conversion from the starting material to a pre-reactant having the structure of Formula VIII can be achieved in a single step. In some forms, the conversion from the staging material to a pre-reactant of Formula VIII can be achieved in two steps. In some forms, the conversion from the starting material to a pre-reactant of Formula VIII can be achieved in three steps. In some forms, the conversion from the starting material to a pre-reactant of Formula VIII can be achieved in four steps. In some forms, the conversion from the starting material to a pre-reactant of Formula VIII can be achieved in five steps. In some forms, the conversion from the starting material to a pre-reactant of Formula VIII can be achieved in six steps.

In some forms, the conversion from the starting material to the pre-reactant of Formula VIII′ can include (i-b1) converting a first starting material to a first intermediate and (i-b2) converting the first intermediate to the pre-reactant having the structure of Formula VIII′ (a first synthetic route).

In some alternative forms, the conversion from the starting material to the pre-reactant of Formula VIII′ can include (i-b1′) converting phloroglucinol (as the starting material) to a second intermediate and (i-b2′) converting the second intermediate to the pre-reactant having the structure of Formula VIII′ (a second synthetic route).

### 1. First Synthetic Route

In some forms, the method described herein can include (i-b1) converting a first starting material having the structure of Formula X to a first intermediate having the structure of Formula IX and (i-b2) converting the first intermediate having the structure of Formula IX to the pre-reactant having the structure of Formula VIII′:

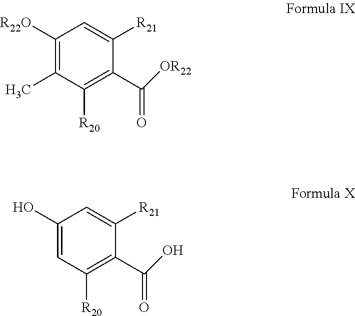

wherein: (a) R₂₀ and R₂₁ can be independently a hydrogen, a hydroxyl, a substituted or unsubstituted alkyl, a carbonyl, an alkoxy, a thiol, or an amino; (b) R₂₂ can be a hydrogen or a substituted or unsubstituted alkyl; and (c) the substituents can be as defined above for Formula I. In some forms, R₂₀ and R₂₁ can be hydroxyl.

Generally, each of steps (i-b1) and (i-b2) can be performed in a suitable solvent at a suitable temperature for a period of time sufficient to form a product containing the desired compound. In some forms, the reaction conditions for performing the reactions in steps (i-b1) and (i-b2) are mild, such as at a temperature <30° C. and a time period ≤24 hours. The solvent suitable for use in each of the steps (i-b1) and (i-b2) can be any of the solvents described above for step (i), such as dimethylsufate, acetone, DMF, butyryl chloride, dichloroethane, nitrobenzene, dioxane, and water, and a combination thereof

a. Converting a Starting Material to an Intermediate

In some forms, the conversion from the first starting material of Formula X to the first intermediate of Formula IX (i.e. step (i-b1)) can include a methylation step and/or an acylation step. In some forms, the conversion from the first starting material of Formula X to the first intermediate of Formula IX can include a methylation step and an acylation step. In these forms, the methylation step can be performed prior to the acylation step.

In some forms, in the methylation step of step (i-b1), the starting material can be converted a methylated compound having the structure of Formula XI′:

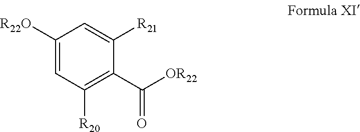

wherein (a) R₂₀ and R₂₁ can be independently a hydrogen, a hydroxyl, a substituted or unsubstituted alkyl, a carbonyl, an alkoxy, a thiol, or an amino; (b) R₂₂ can be a hydrogen or a substituted or unsubstituted alkyl; and (c) the substituents can be as defined above for Formula I.

In some forms, in the acylation step of step (i-b1), the methylated compound of Formula XI′ can be converted to an acylated compound having the structure of Formula XI:

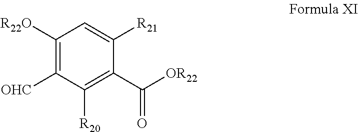

wherein (a) R₂₀ and R₂₁ can be independently a hydrogen, a hydroxyl, a substituted or unsubstituted alkyl, a carbonyl, an alkoxy, a thiol, or an amino; (b) R₂₂ can be a hydrogen or a substituted or unsubstituted alkyl; and (c) the substituents can be as defined above for Formula I.

In some forms, the conversion from the first starting material of Formula X to the first intermediate of Formula IX further includes a reduction step (i.e. step (i-b3)), in which the acylated compound having the structure of Formula XI can be converted to the first intermediate having the structure of Formula IX. In some forms, the coversion of the acylated compound of Formula XI to the first intermediate of Formula IX is via a Vilsmeier-Haack reaction.

Each of the steps/reactions for converting the first starting material of Formula X to the first intermediate of Formula IX can be performed under the conditions described above. For example, the temperature for performing each reaction in step (i-b1) can be up to 35° C., up to 30° C., up to 25° C., in a range from 0° C. to 35° C., from ° C. to 30° C., or from 0° C. to 25° C., at 1 atm, such as from 0° C. to room temperature (i.e. from about 20° C. to about 22° C., at 1 atm) or room temperature; the time period for performing each reaction in step (i-b1) can be up to 24 hours, up to 20 hours, up to 18 hours, or up to 16 hours, in a range from about 4 hours to about 24 hours, from about 5 hours to about 20 hours, from about 6 hour to about 18 hours, or from about 8 hour to about 16 hours, such as about 3 hours or about 18 hours; and optionally the reaction can be performed in the presence of a suitable catalyst, such as POCl₃.

More specific conditions and reagents for converting an exemplary first starting material to an exemplary first intermediate are described in the Examples below.

b. Converting an Intermediate to a Pre-Reactant

In some forms, the conversion of the first intermediate of Formula IX to the pre-reactant of Formula VIII′ (i.e. step (i-b2)) can include an acylation step and a decarboxylation step, preformed sequentially. Exemplary steps in the conversion from the first intermediate of Formula IX to the pre-reactant of Formula VIII′ are:

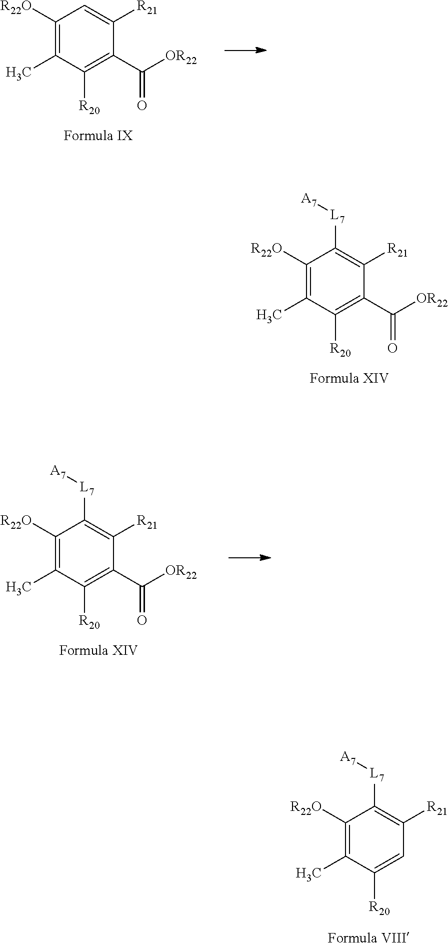

Each of the steps/reactions for converting the first intermediate of Formula IX to the pre-reactant of Formula VIII′ can be performed under the conditions described above. For example, the temperature for performing each reaction in step (i-b2) can be up to 90° C., up to 85° C., up to 80° C., in a rang from 60° C. to 90° C., from 60° C. to 85° C., or from 70° C. to 90° C., at 1 atm, such as about 70° C. or about 80° C., at 1 atm; the time period for performing each reaction in step (i-b2) can be up to 24 hours, up to 20 hours, up to 18 hours, or up to 16 hours, in a range from about 4 hours to about 24 hours, from about 5 hours to about 20 hours, from about 6 hour to about 18 hours, or from about 8 hour to about 16 hours, such as about 5 hours or about 16 hours; and optionally the reaction can be performed under reflux and/or in the presence of a suitable catalyst, such as a Lewis acid, e.g., AlCl₃, ZnCl₂, FeCl₃, etc.

More specific conditions and reagents for converting an exemplary first intermediate compound to an exemplary pre-reactant are described in the Examples below.

### 2. Second Synthetic Route

In some alternative forms, the method described herein can include (i-b1′) converting phloroglucinol (as the starting material) to a second intermediate having the structure of Formula XVII and (i-b2′) converting the second intermediate having the structure of Formula XVII to the pre-reactant having the structure of Formula VIII′:

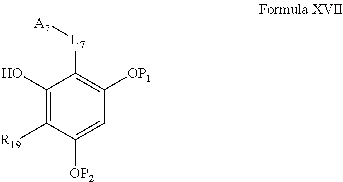

wherein: (a) L₇ can be a bond (single, double, or triple), a substituted or unsubstituted alkyl, or a carbonyl; (b) A₇ can be a hydrogen, a substituted or unsubstituted alkyl, a substituted or unsubstituted aryl, a carbonyl, or an alkoxy; (c) R₁₉ can be a hydrogen, a hydroxyl, a substituted or unsubstituted alkyl, a carbonyl, an alkoxy, a thiol, or an amino; (d) P₁ and P₂ can be independently a protection group, such as a methoxymethyl ether, tetrahydropyranyl ether, butyl ether, allyl ether, acetonide, benzylidene acetal, acetic acid esters, pivalic acid ester, tert-butyldimethylsilyl ethers, tert-butyldibenzylsilyl ethers, benzyl ether, benzoic acid ester, benzoate ester, benzoate, etc.; and (e) the substituents can be as defined above for Formula I. In some forms, P₁ and P₂ can be methoxymethyl ether (MOM).

In some forms, R₁₉ can be a substituted or unsubstituted alkyl, such as an unsubstituted C₁-C₁₀ alkyl, an unsubstituted C₁-C₈ alkyl, an unsubstituted C₁-C₆ alkyl, an unsubstituted C₁-C₄ alkyl, an unsubstituted C₁-C₃ alkyl, or an unsubstituted C₁-C₂ alkyl, such as an unsubstituted methyl.

Generally, each of steps (i-b1′) and (i-b2′) can be performed in a suitable solvent at a suitable temperature for a period of time sufficient to form a product containing the desired compound. In some forms, the reaction conditions for performing the reactions in steps (i-b1′) and (i-b2′) are mild, such as at a temperature <90° C. and a time period ≤15 hours. The solvent suitable for use in each of the steps (i-b1′) and (i-b2′) can be any of the solvents described above for step (i), such as 2-methylbutyryl chloride, dichloroethane, nitrobenze, DMF, THF, butyryl chloride, acetone, dimethyl sulfate, acetone, methanol, CHCl₃, and toluene, and a combination thereof.

a. Converting Phloroglucinol to an Intermediate

In some forms, the conversion from phloroglucinol to the second intermediate of Formula XVII (i.e. step (i-b1′)) can include an acylation step. In some forms, the conversion from phloroglucinol to the second intermediate of Formula XVII can include an oxidation step, a reduction step, and an acylation step, performed sequentially. In some forms, the conversion from phloroglucinol to the second intermediate of Formula XVII can include an oxidation step, a reduction step, an acylation step, and a protection step, performed sequentially. Exemplary steps in the conversion from phloroglucinol to an exemplary second intermediate are shown below.

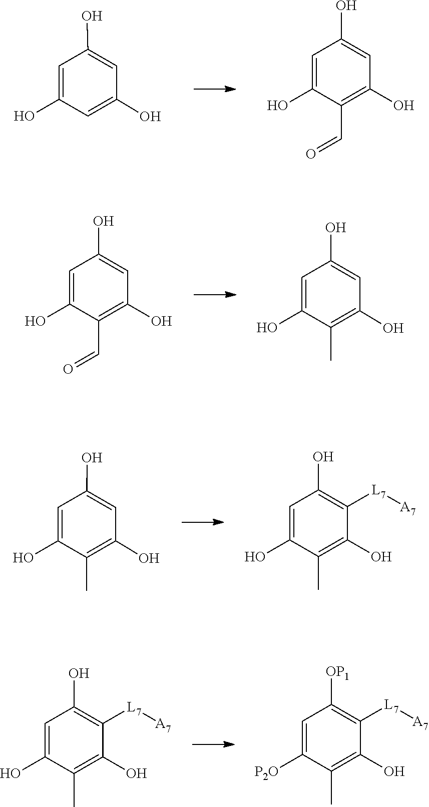

Each of the steps/reactions for converting phloroglucinol to the second intermediate of Formula XVII can be performed under the conditions described above. For example, the temperature for performing each reaction in step (i-b1′) can be up to 90° C., up to 85° C., up to 80° C., in a rang from 20° C. to 90° C., from 40° C. to 90° C., from 50° C. to 90° C., from 60° C. to 90° C., or from 70° C. to 90° C., at 1 atm, such as about 80° C. or at room temperature, at 1 atm; the time period for performing each reaction in step (i-b1′) can be up to 15 hours, up to 12 hours, up to 10 hours, or up to 4 hours, in a range from about 1 hour to about 15 hours, from about 2 hours to about 15 hours, from about 3 hour to about 15 hours, or from about 1 hour to about 4 hours, such as about 1 hour, about 3 hours, about 4 hours, or about 15 hours; and optionally the reaction can be performed in the presence of a suitable catalyst, such as a Lewis acid, e.g., AlCl₃, POCl₃, ZnCl₂, FeCl₃, etc.

More specific conditions and reagents for converting phloroglucinol to an exemplary second intermediate are described in the Examples below.

b. Converting an Intermediate to a Pre-Reactant

In some forms, the conversion from the second intermediate of Formula XVII to the pre-reactant of Formula VIII′ (i.e. step (i-b2′)) can include an acylation step and optionally a deprotection step. In some forms, in the acylation step of step (i-b2′), the second intermediate of Formula XVII can be converted to an acylated compound having the structure of Formula XVIII:

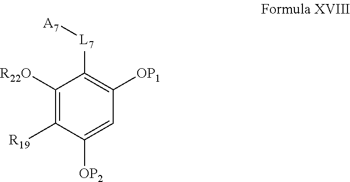

wherein: (a) L₇ can be a bond (single, double, or triple), a substituted or unsubstituted alkyl, or a carbonyl; (b) A₇ can be a hydrogen, a substituted or unsubstituted alkyl, a substituted or unsubstituted aryl, a carbonyl, or an alkoxy; (c) R₁₉ can be a hydrogen, a hydroxyl, a substituted or unsubstituted alkyl, a carbonyl, an alkoxy, a thiol, or an amino; (d) R₂₂ can be a hydrogen or a substituted or unsubstituted alkyl; (e) P₁ and P₂ can be independently a protection group as described above for Formula XVII; and (f) the substituents can be as defined above for Formula I.

This acylation reaction can be performed at a temperature of up to 90° C., in a range from 60° C. to 90° C., from 60° C. to 85° C., or from 60° C. to 80° C., at 1 atm, such as about 70° C., at 1 atm; for a time period of up to 15 hours, up to 12 hours, in a range from about 5 hour to about 15 hours, from about 8 hours to about 15 hours, from about 10 hour to about 15 hours, or from about 12 hour to about 15 hours; and optionally the reaction can be performed under reflux.

The acylated compound of Formula XVIII can then be converted to the pre-reactant of Formula VIII′ after removing the protection groups of —OP₁ and —OP₂. Reaction conditions for removing the protection groups from a compound are known. For example, the protection groups of the acylated compound of Formula XVIII can be removed in an acidic solvent, such as 1 M HCl in methanol, at a temperature of up to 85° C., in a range from 65° C. to 85° C., from 65° C. to 80° C., from 65° C. to 75° C., or from 65° C. to 70° C., at 1 atm, such as about 65° C., at 1 atm, for a time period of up to 2 hours, up to 1 hour, in a range from 10 minutes to 2 hours, from 10 minutes to 1 hours, from 15 minutes to 1 hour, or from 20 minutes to 1 hour, such as about 1 hour, and optionally the reaction can be performed under reflux.

More specific conditions and reagents for converting an exemplary second intermediate to an exemplary pre-reactant are described in the Examples below.

### C. Yield

Generally, the AGB or a derivative thereof in the product formed using the disclosed method can have an overall yield of at least 7%, at least 10%, at least 12%, at least 15%, at least 20%, at least 25%, at least 30%, in a range from about 20% to about 75%, from about 20% to about 70%, from about 20% to about 60%, from about 20% to about 50%, from about 30% to about 75%, from about 30% to about 70%, from about 30% to about 60%, or from about 30% to about 50%. The overall yield of AGB or its derivative in the product is calculated from the starting material using the cumulative yield from each step. The experimentally obtained total no. of mole of the AGB or its derivative can be determined using known methods, such as using NMR (e.g. ¹H NMR, ¹⁹F NMR, and/or ³¹P NMR) spectroscopy with an internal standard of known quantity, such as using a known quantity of CDCl₃-d or MeOD-d4 as the internal standard.

In some forms, the AGB or AGB derivative of any of Formulae I, Ia, and II-IV in the product formed using the disclosed methods can have an overall yield of at least 7%, at least 10%, at least 12%, at least 15%, at least 20%, at least 25%, at least 30%, in a range from about 20% to about 75%, from about 20% to about 70%, from about 20% to about 60%, from about 20% to about 50%, from about 30% to about 75%, from about 30% to about 70%, from about 30% to about 60%, or from about 30% to about 50%.

In some forms, the AGB or AGB derivative of any of Formulae I, Ia, and II-IV in the product formed from a starting material of Formula X using the disclosed methods, such as one that includes the first synthetic route described above, can have an overall yield of at least 7%, at least 10%, at least 12%, at least 15%, in a range from about 7% to about 75%, from about 7% to about 50%, from about 7% to about 40%, from about 7% to about 30%, from about 7% to about 25%, or from about 7% to about 20%.

In some forms, the AGB or AGB derivative of any of Formulae I, Ia, and II-IV in the product formed from phloroglucinol as the starting material using the disclosed methods, such as one that includes the second synthetic route described above, can have an overall yield of at least 20%, at least 25%, at least 30%, in a range from about 20% to about 75%, from about 20% to about 70%, from about 20% to about 60%, from about 20% to about 50%, from about 30% to about 75%, from about 30% to about 70%, from about 30% to about 60%, or from about 30% to about 50%.

Exemplary methods for synthesizing AGB and exemplary AGB derivatives and their corresponding yield, are described in Example below.

### D. Optional Steps

The disclosed methods can include one or more optional steps, such as mixing the first reactant, the second reactant, the third reactant, and the solvent to form the reaction mixture prior to step (i); stirring the reaction mixture prior to and/or during step (i); and/or purifying the product, optionally by flash column chromatography, subsequent to step (i).

### 1. Stirring the Reaction Mixture

Optionally, the reaction mixture is under stirring prior to and/or during step (i), and/or under refluxing during step (i), heating the reaction mixture at a temperature for a period of time sufficient to form a product containing AGB or a derivative thereof. For example, the reaction mixture is stirred and/or refluxed at the reaction temperature during the entire reaction period to form the product containing AGB or a derivative thereof. Techniques for stirring and refluxing the reaction mixture during reaction are known, such as by using a mechanical stirring bar, magnetic stirring beads, etc. for stirring and using a condenser for refluxing.

### 2. Purifying the Product

Optionally, the disclosed method includes a step of purifying the product to remove impurities, such as unreacted reactants, in the product, and thereby obtain isolated AGB or its derivative, subsequent to reaction in step (i). The product can be purified by known methods, such as using flash column chromatography on silica gel or recrystallization in a dichloromethane/hexane mixture.

### IV. Methods for Using AGB and Derivatives Thereof

The disclosed compounds have anticancer properties and thereby can be used in methods for treating a cancer, reducing a cancer, or treating or ameliorating one or more symptoms associated with a cancer in a subject in need thereof. In some forms, the compounds can be used in methods for treating cancer cells and/or cancer stem cells in a subject in need thereof.

A. Treating Cancer, Reducing Cancer, or Treating or Ameliorating Symptom(s) Associated with Cancer

Methods of using the compounds for treating a cancer, reducing a cancer, or treating or ameliorating one or more symptoms associated with a cancer in a subject in need thereof are disclosed.

Generally, the method includes (i) administering to the subject a pharmaceutical formulation containing one or more of the compounds described above. The administration step can occur one or more times.

The subject can be a mammal, such as a human, a dog, a cat, a rat, a monkey, rabbits, guinea pigs, etc., that is in need of cancer treatment. In some forms, the subject can be exhibiting symptoms of or diagnosed with cancer.

The pharmaceutical formulation can be administered by oral administration, parenteral administration, inhalation, mucosal administration, topical or a combination thereof. The compound(s) can be administered by a medical professional or the subject being treated (e.g. self-administration).

### 1. Cancers

The cancer being treated using the disclosed methods can be tumors, such as tumors of the hematopoietic and lymphoid tissues or hematopoietic and lymphoid malignancies, tumors that affect the blood, bone marrow, lymph, and lymphatic system, and tumors located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax, and genito-urinary apparatus.

In some forms of the method, the cancer can be a lung cancer, such as a non-small-cell lung cancer.

In some forms of the method, the cancer can be a colon cancer, breast cancer, ovarian cancer, cervical cancer, lung cancer, rectal cancer, kidney cancer, liver cancer, brain cancer, or leukemia, or a combination thereof. In some forms of the method, the cancer can be breast cancer, such as triple negative breast cancer (TNBC).

In some forms of the method, the cancer can be AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the oesophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, tricoleukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

### 2. Effective Amount/Dosage

Generally, following the administration step of the disclosed method, the pharmaceutical formulation is administered in an effective amount to treat a cancer, reduce a cancer, or treat or ameliorate one or more symptoms associated with a cancer in a subject.

In some forms, the pharmaceutical formulation is administered in an effective amount to inhibit the growth of a tumor by at least 40%, at least 50%, or at least 55% in the subject compared to the same tumor in a control subject. Whether tumor growth is inhibited may be identified by a variety of diagnostic manners known to one skill in the art including, but not limited to, observation of the reduction in size or number of tumor masses, in comparison with the same type of tumor in a control subject, using imaging, by the end of a certain time period following administration of the effective amount of pharmaceutical formulation. Imaging suitable for measuring the size or number of tumor masses include, but are not limited to, transrectal ultrasound, MRI, computerized tomography (“CT”) scan, positron emission tomography (“PET”) imaging, multiparametric ultrasound (“US”), or a combination thereof, for example, PET-CT, PET-MRI, MRI-US, etc.

In some forms, the pharmaceutical formulation is administered in an effective amount to reduce the level of a biomarker associated with a cancer in the blood of the subject by at least 5%, at least 10%, at least 15%, at least 20%, at least 25%, or at least 30% compared to the level of the biomarker in the blood of the subject before treatment. For example, the pharmaceutical formulation is administered in an effective amount to reduce the level of a biomarker associated with a lung cancer (e.g. carcinoembryonic antigen (CEA), squamous cell carcinoma antigen (SCC), neuron-specific enolase (NSE), cytokeratin 19 fragment (CYFRA), and pro-gastrin-releasing peptide (proGRP)) in the blood of the subject by at least 5%, at least 10%, at least 15%, at least 20%, at least 25%, or at least 30% compared to the level of the biomarker in the blood of the subject before treatment.

Administering an effective amount of the pharmaceutical formulation can be achieved in a single administration step or using multiple administration steps. For example, if the unit dosage form contains an effective amount of the compound(s) to inhibit the growth of a tumor by at least 40%, at least 50%, or at least 55% in the subject and/or reduce the level of a biomarker associated with a cancer in the blood of the subject, then the method only requires a single administration step. Alternatively, if the unit dosage form contains less than the required effective amount of the compound(s) to inhibit the growth of a tumor by at least 40%, at least 50%, or at least 55% in the subject and/or induce apoptosis of cancer cells, then the method involves at least two steps of administering the pharmaceutical formulation, and optionally more than two steps of administering the pharmaceutical formulation to the subject until an effective amount of the pharmaceutical formulation is administered to the subject to inhibit the growth of a tumor by at least 40%, at least 50%, or at least 55% in the subject and/or to reduce the level of a biomarker associated with a cancer in the blood of the subject. When multiple administration steps are needed to administer an effective amount of the pharmaceutical formulation to the patient, each administration step may administer the same dosage or different dosages of the pharmaceutical formulation to the patient.

When multiple administration steps are needed to administer an effective amount of the pharmaceutical formulation to the patient, the administration steps may be performed regularly or irregularly. For example, the administration steps are performed at a suitable frequency, such as every hour, every 2 hours, every 5 hours, every 8 hours, every day, every 2 days, every 3 days, every 5 days, every 7 days, every 10 days, every two weeks, or every month. For example, the administration step is performed every hour, every 2 hours, every 5 hours, every 8 hours, every day, every 2 days, every 3 days, every 5 days, every 7 days, every 10 days, every two weeks, or every month for a period between one day and 6 months, between one day and 3 months, between one and thirty days, between one and ten days, between one and three days, between one and two days, or for one day. Alternatively, the administration may be performed irregularly, for example, the administration step is performed 1 day after the first administration, then 2 days after the second administration, then 5 days after the third administration, then 7 day after the fourth administration, and then 30 days after the fifth administration. The time interval between administrations is determined based on the patient's needs.

In some forms, the method includes only a single administration of the pharmaceutical formulation, wherein the pharmaceutical formulation contains an effective amount of the compounds to inhibit the growth of a tumor by at least 40%, at least 50%, or at least 55% in the subject compared to the same tumor in a control subject, and/or to reduce the level of a biomarker associated with a cancer in the blood of the subject compared to the level of the biomarker in the blood of the subject before treatment.

In some forms, the method includes more than one step of administering to the subject the pharmaceutical formulation, wherein following all of the administration steps an effective amount of the compounds to inhibit the growth of a tumor by at least 40%, at least 50%, or at least 55% in the subject compared to the same tumor in a control subject, and/or to reduce the level of a biomarker associated with a cancer in the blood of the subject compared to the level of the biomarker in the blood of the subject before treatment, is administered to the subject.

In some forms, following a single administration or multiple administrations, the effective amount of compound(s) that is administered to the subject to treat a cancer, reduce a cancer, or treat or ameliorate one or more symptoms associated with a cancer in a subject, such as to inhibit the growth of a tumor by at least 40%, at least 50%, or at least 55% in the subject compared to the same tumor in a control subject, and/or to reduce the level of a biomarker associated with a cancer in the blood of the subject compared to the level of the biomarker in the blood of the subject before treatment, can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 3 mg/kg of the subject.

### 3. Optional Steps

a. Administering Additional Active Agent(s)

One or more active agents in addition to the compounds may be administered to the subject throughout the method or at different intervals during the method. For example, the one or more additional active agents is administered to the subject prior to, during, and/or subsequent to step (i). In some forms, the one or more additional active agents can be included in a pharmaceutical formulation containing the compound(s) and is administered to the subject simultaneously with the compound(s) in the pharmaceutical formulation in association with one or more pharmaceutically acceptable excipients. In some forms, the one or more additional active agents can be administered separately from the pharmaceutical formulation containing the compound(s).

In some forms, the one or more additional active agents are one or more anticancer agents described above. The amount of the one or more additional anticancer agents required will vary from subject to subject according to their need.

B. Treating Cancer Cells and/or Cancer Stem Cells

In some forms, the compounds can be used in a method for treating cancer cells and/or cancer stem cells in a subject in need thereof.

The method can follow the method step described above, for example, administering to the subject the pharmaceutical formulation containing the compound(s), such as by oral administration, parenteral administration, inhalation, mucosal, topical administration, or a combination thereof. The administration step can occur one or more times to administer an effective amount of the compound(s) in the pharmaceutical formulation to treat cancer cells and/or cancer stem cells, depending on whether a unit dosage contains an effective amount of the compound(s) to to treat cancer cells and/or cancer stem cells. When multiple administrations are needed to achieve a required effective amount of the compound(s) in the subject, the dosage and frequency for each administration can follow the method described above.

In some forms, the method can include the additional step described above. For example, the user can administer one or more additional active agents to the subject prior to, during, and/or subsequent to administering the compound to the subject.

In some forms of the method, the compound(s) can have an IC₅₀ value against the cancer cells or cancer stem cells lower than an IC₅₀ value of the same compound against non-cancerous cells, tested under the same condition. The cancer cells and/or cancer stem cells being treated in the subject can be the cancer cells of any one of the cancers described above. For example, the cancer cells can be MDA-MB-231, MCF-7, HepG2, Hela, AGS, HTC116, SW480, SUNE-1, H460 (e.g. NCI-H460, stem-like NCI-H460, etc.), HCC827, H1650, or A549, or a combination thereof. In some forms, the cancer cells can be NCI-H460, stem-like NCI-H460, or A549, or a combination thereof.

When comparing the IC₅₀ values of the compound against cancer cells or cancer stem cells with the IC₅₀ values of the same compound against non-cancerous cells, the non-cancerous cells can be from any normal tissue of the subject, such as CCD-19Lu.

### 1. Effective Amount/Dosage

Generally, following the administration step of the disclosed method, the pharmaceutical formulation is administered in an effective amount to treat cancer cells and/or cancer stem cells in a subject.

In some forms, following the administration step of the disclosed method, the pharmaceutical formulation is administered in an effective amount to induce mitochondria dysfunction and/or to inhibit ER to Golgi trafficking in cancer cells in the subject, compared to the subject before administered with the pharmaceutical formulation.

In some forms, following the administration step of the disclosed method, the pharmaceutical formulation is administered in an effective amount to induce apoptosis of cancer cells in the subject, compared to the subject before administered with the pharmaceutical formulation.

Methods for determining whether mitochondria disfunction is induced, whether ER to Golgi trafficking is inhibited, and whether apoptosis of cancer cells is induced are described above.

In some forms, the method includes only a single administration of the pharmaceutical formulation, wherein the pharmaceutical formulation contains an effective amount of the compounds to induce mitochondria dysfunction, to inhibit ER to Golgi trafficking, and/or to induce apoptosis of cancer cells in the subject, compared to the subject before administered with the pharmaceutical formulation, as determined using one or more parameters described above.

In some forms, the method includes more than one step of administering to the subject the pharmaceutical formulation, wherein following all of the administration steps an effective amount of the compounds to to induce mitochondria dysfunction, to inhibit ER to Golgi trafficking, and/or to induce apoptosis of cancer cells in the subject, compared to the subject before administered with the pharmaceutical formulation, as determined using one or more parameters described above, is administered to the subject.

In some forms, following a single administration or multiple administrations, the effective amount of compound(s) that is administered to the subject to treat cancer cells and/or stem like cancer cells in a subject, such as to induce mitochondria dysfunction, to inhibit ER to Golgi trafficking, and/or to induce apoptosis of cancer cells in the subject, compared to the subject before administered with the pharmaceutical formulation, as determined using one or more parameters described above, can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 3 mg/kg of the subject.

The disclosed compounds and methods can be further understood through the following enumerated paragraphs.

1. A compound having the structure of

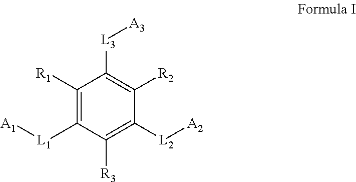

wherein:
(a) L₁ and L₂ are independently a bond (single, double, or triple), a substituted or unsubstituted alkyl, a substituted or unsubstituted alkenyl, a carbonyl, or an alkoxy;
(b) L₃ is a bond (single, double, or triple), a substituted or unsubstituted alkyl, or a carbonyl;
(c) A₁ and A₂ are independently a hydrogen, a substituted or unsubstituted alkyl, a substituted or unsubstituted alkenyl, a substituted or unsubstituted aryl, a carbonyl, or an alkoxy, and at least one of A₁ and A₂ is a substituted or unsubstituted aryl;
(d) A₃ is a hydrogen, a substituted or unsubstituted alkyl, a substituted or unsubstituted aryl, a carbonyl, or an alkoxy;
(e) R₁—R₃ are independently a hydrogen, a hydroxyl, a substituted or unsubstituted alkyl, a carbonyl, an alkoxy, a thiol, or an amino; and
(f) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

2. The compound of paragraph 1, wherein A₁ and/or A₂ are independently a substituted or unsubstituted aryl.

3. The compound of paragraph 1 or paragraph 2, wherein the compound has the structure of

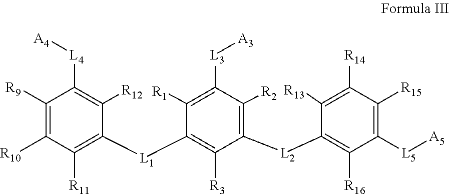

wherein:
(a) L₁ and L₂ are independently a bond (single, double, or triple), a substituted or unsubstituted alkyl, a substituted or unsubstituted alkenyl, a carbonyl, or an alkoxy;
(b) L₃-L₅ are independently a bond (single, double, or triple), a substituted or unsubstituted alkyl, or a carbonyl;
(c) A₃-A₅ are independently a hydrogen, a substituted or unsubstituted alkyl, a substituted or unsubstituted aryl, a carbonyl, or an alkoxy;
(d) R₁—R₃ and R₉—R₁₆ are independently a hydrogen, a hydroxyl, a substituted or unsubstituted alkyl, a carbonyl, an alkoxy, a thiol, or an amino; and
(e) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

4. The compound of any one of paragraphs 1-3, wherein -L₁-A₁ is the same as -L₂-A₂, and wherein the compound has the structure of

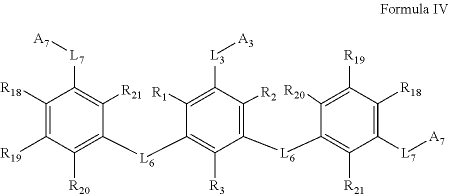

wherein:
(a) L₃ and L₇ are independently a bond (single, double, or triple), a substituted or unsubstituted alkyl, or a carbonyl;
(b) L₆ is a bond (single, double, or triple), a substituted or unsubstituted alkyl, a substituted or unsubstituted alkenyl, a carbonyl, or an alkoxy;
(c) A₃ and A₇ are independently a hydrogen, a substituted or unsubstituted alkyl, a substituted or unsubstituted aryl, a carbonyl, or an alkoxy;
(d) R₁—R₃ and R₁₈—R₂₁ are independently a hydrogen, a hydroxyl, a substituted or unsubstituted alkyl, a carbonyl, an alkoxy, a thiol, or an amino; and
(e) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

5. The compound of any one of paragraphs 1-4, wherein R₁—R₃, R₈—R₁₆, and/or R₁₈—R₂₁ are independently a hydroxyl, a substituted or unsubstituted alkyl, —OR₄, —SR₅, or —NR₆R₇, R₄—R₇ are independently a hydrogen or a substituted or unsubstituted alkyl.

6. The compound of any one of paragraphs 1-5, wherein R₁—R₃ are hydroxyl.

7. The compound of any one of paragraphs 1-6, wherein R₈—R₁₆ and/or R₁₈—R₂₁ are independently a hydroxyl, a substituted or unsubstituted alkyl, or —OR₄, R₄ is a substituted or unsubstituted alkyl.

8. The compound of any one of paragraphs 1-7, wherein -L₃-A₃ is a substituted or unsubstituted C₁-C₆ alkyl, or

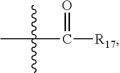

R₁₇ is a substituted or unsubstituted C₁-C₆ alkyl, a substituted or unsubstituted aryl, or

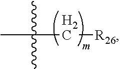

m can be an integer from 0 to 5 and R₂₆ can be a carboxylic acid, an ester, an amino, or an amide.

9. The compound of paragraph 8, wherein R₁₇ is a substituted or unsubstituted C₁-C₅ alkyl (e.g. an unsubstituted C₁-C₅ alkyl), a substituted or unsubstituted C₁-C₄ alkyl (e.g. an unsubstituted C₁-C₄ alkyl), a substituted or unsubstituted C₁-C₃ alkyl (e.g. an unsubstituted C₁-C₃ alkyl), a substituted or unsubstituted C₁-C₂ alkyl (e.g. an unsubstituted C₁-C₂ alkyl), or a substituted or unsubstituted methyl (e.g. an unsubstituted methyl).

10. The compound of paragraph 8, wherein R₁₇ is a C₆ cycloalkyl.

11. The compound of paragraph 8, wherein R₁₇ is an unsubstituted phenyl.

12. The compound of paragraph 8, wherein R₂₆ is an ester.

13. The compound of any one of paragraphs 1-12, wherein -L₄-A₄, -L₅-A₅, and/or -L₇-A₇ are independently

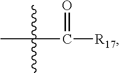

R₁₇ is a substituted or unsubstituted C₁-C₆ alkyl.

14. The compound of claim 1 or 2, wherein -L₁-A₁ is different from -L₂-A₂, and wherein the compound has the structure of

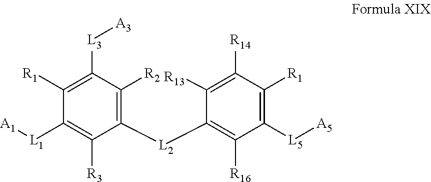

wherein
(a) L₁ and L₂ are independently a single bond, a substituted or unsubstituted alkylene, a substituted or unsubstituted alkenylene, —C(O)—, or an alkoxy;
(b) A₁ is a substituted or unsubstituted alkyl, a substituted or unsubstituted alkenyl, a substituted or unsubstituted aryl, a carbonyl, or an alkoxy;
(c) R₁—R₃, and/or R₁₃—R₁₆ are independently a hydrogen, a hydroxyl, a substituted or unsubstituted alkyl, —OR₄, —SR₅, or —NR₆R₇, R₄—R₇ are independently a hydrogen or a substituted or unsubstituted alkyl;
(d) -L₃-A₃ is

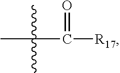

R₁₇ is a substituted or unsubstituted alkyl, a substituted or unsubstituted aryl; and
(e) -L₅-A₅ is a hydrogen, a substituted or unsubstituted alkyl, or

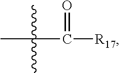

R₁₇ is a substituted or unsubstituted alkyl.

15. The compound of paragraph 1 or 2, wherein -L₁-A₁ is different from -L₂-A₂, and wherein the compound has the structure of

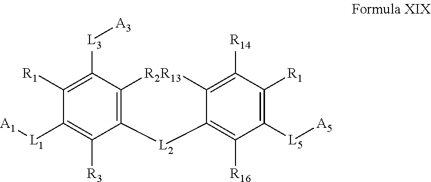

wherein
(a) L₁ and L₂ are independently a substituted or unsubstituted alkylene, or —C(O)—;
(b) A₁ is a substituted or unsubstituted aryl, or an C₁-C₆ alkoxy;
(c) R₁—R₃ are a hydroxyl;
(d) R₁₃—R₁₆ are independently a hydroxyl, a hydrogen, a substituted or unsubstituted alkyl, or —OR₄, R₄ is a substituted or unsubstituted C₁-C₆ alkyl;
(e) -L₃-A₃ is

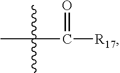

R₇ is a substituted or unsubstituted C₁-C₆ alkyl, a substituted or unsubstituted aryl; and
(f) -L₅-A₅ is a hydrogen, a substituted or unsubstituted C₁-C₆ alkyl, or

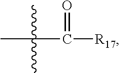

R₁₇ is a substituted or unsubstituted C₁-C₆ alkyl.

16. The compound of paragraph 15, wherein A₁ is an alkoxy substituted phenyl.

17. The compound of paragraph 15, wherein A₁ is an unsubstituted phenyl.

18. The compound of any one of paragraphs 15-17, wherein -L₃-A₃ is

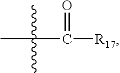

R₁₇ is an unsubstituted phenyl

19. The compound of paragraph 1 or 2, wherein -L₁-A₁ is different from -L₂-A₂, and wherein the compound has the structure of

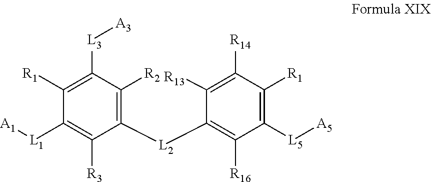

wherein
(a) L₁ and L₂ are independently a substituted or unsubstituted alkylene, or —C(O)—;
(b) A₁ is an C₁-C₆ alkoxy;
(c) R₁—R₃ are a hydroxyl;
(d) R₁₃—R₁₆ are a hydroxyl, a substituted or unsubstituted C₁-C₆ alkyl, or —OR₄, R₄ is a substituted or unsubstituted C₁-C₆ alkyl;
(e) -L₃-A₃ is

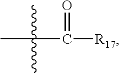

R₁₇ is a substituted or unsubstituted C₁-C₆ alkyl; and
(f) -L₅-A₅ is

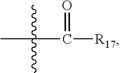

R₁₇ is a substituted or unsubstituted C₁-C₆ alkyl.

20. The compound of any one of paragraphs 1-19, wherein the compound is not

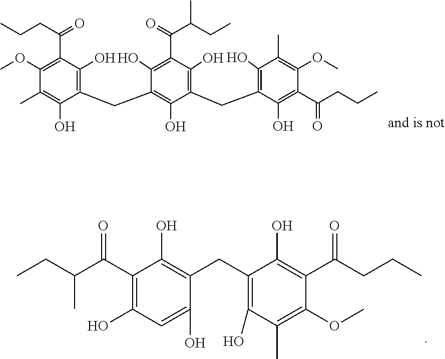

21. The compound of any one of paragraphs 1-20, wherein the compound is

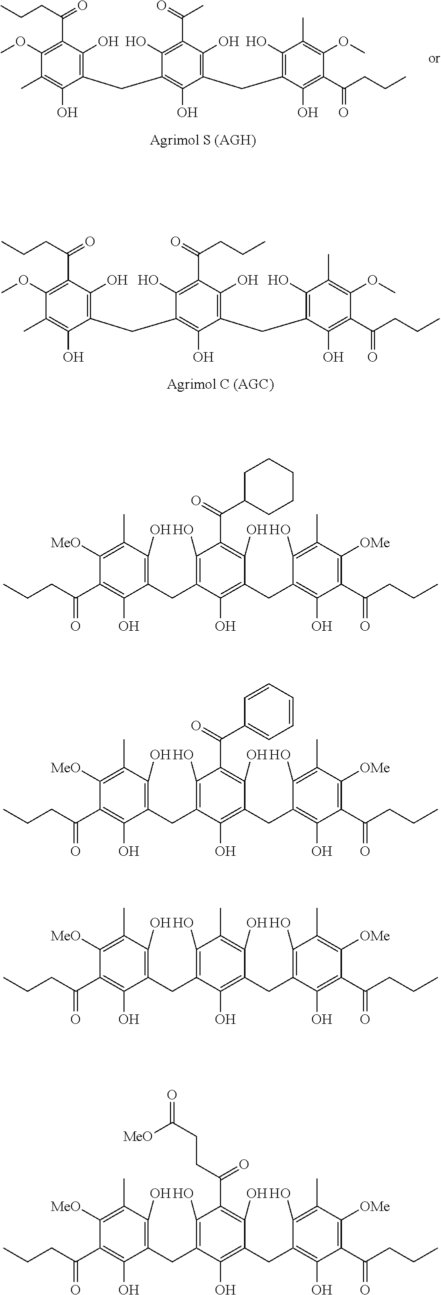

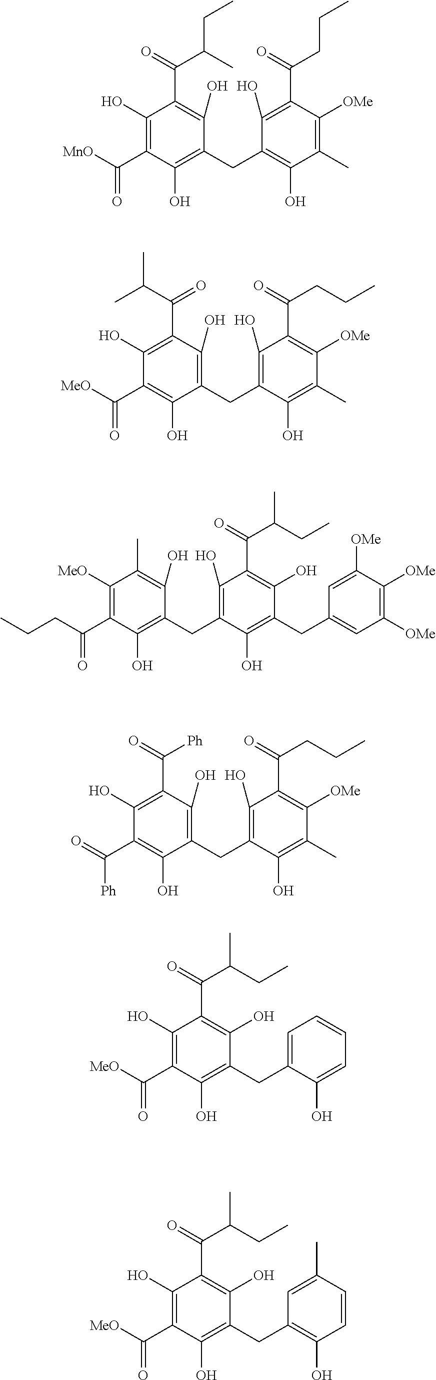

22. A pharmaceutical formulation comprising
one or more compounds of any one of paragraphs 1-21; and
a pharmaceutically acceptable carrier and/or excipient.

23. The pharmaceutical formulation of paragraph 22, wherein the one or more compounds are in an effective amount to treat a cancer, reduce a cancer, or treat or ameliorate one or more symptoms associated with a cancer in a subject.

24. The pharmaceutical formulation of paragraph 23, wherein the effective amount of the one more compounds is effective to induce mitochondria dysfunction and/or to inhibit ER to Golgi trafficking in cancer cells in the subject.

25. The pharmaceutical formulation of paragraph 23 or 24, wherein the effective amount of the one more compounds is effective to induce apoptosis of cancer cells in the subject.

26. The pharmaceutical formulation of any one of paragraphs 23-25, wherein the effective amount of the one more compounds is effective to inhibit the growth of a tumor by at least 40%, at least 50%, or at least 55% in the subject compared to the same tumor in a control subject.

27. The pharmaceutical formulation of any one of paragraphs 22-26, further comprising a second active agent, optionally more than one second active agent, optionally the second active agent is an anticancer agent.

28. The pharmaceutical formulation of any one of paragraphs 22-27, wherein the total concentration of the one or more compounds in the pharmaceutical formulation is at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

29. The pharmaceutical formulation of any one of paragraphs 22-28, in a unit dosage form, wherein the dosage of the compounds is in a range from about 0.002 mg to about 1 mg, in a range from about 0.006 mg to about 0.6 mg, in a range from about 0.01 mg to about 0.4 mg, in a range from about 0.02 mg to about 0.3 mg, or in a range from about 0.01 mg to about 0.2 mg.

30. A method for synthesizing the compound of any one of paragraphs 1-21 comprising:
(i) heating a reaction mixture at a suitable temperature for a period of time sufficient to form a product comprising the compound, wherein the reaction mixture comprises a solvent, a first reactant having the structure of Formula V,

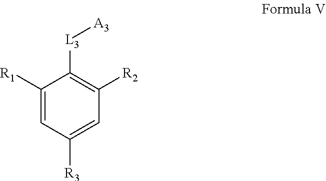

a second reactant having the structure of Formula VI, and

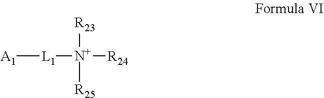

a third reactant having the structure of Formula VII,

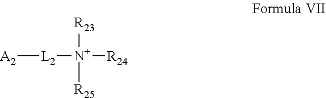

wherein
(a) L₁ and L₂ are independently a bond (single, double, or triple), a substituted or unsubstituted alkyl, a substituted or unsubstituted alkenyl, a carbonyl, or an alkoxy;
(b) L₃ is a bond (single, double, or triple), a substituted or unsubstituted alkyl, or a carbonyl;
(c) A₁ and A₂ are independently a hydrogen, a substituted or unsubstituted alkyl, a substituted or unsubstituted alkenyl, a substituted or unsubstituted aryl, a carbonyl, or an alkoxy, and at least one of A₁ and A₂ is a substituted or unsubstituted aryl;
(d) A₃ is a hydrogen, a substituted or unsubstituted alkyl, a substituted or unsubstituted aryl, a carbonyl, or an alkoxy;
(e) R₁—R₃ are independently a hydrogen, a hydroxyl, a substituted or unsubstituted alkyl, a carbonyl, an alkoxy, a thiol, or an amino;
(f) R₂₃—R₂₅ are independently a hydrogen or a substituted or unsubstituted alkyl; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl, and
wherein the second reactant is the same as or different from the third reactant.

31. The method of paragraph 30, wherein:
A₁ is

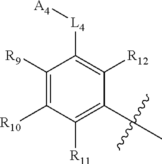

A₂ is

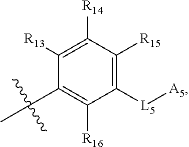

wherein:
(a) L₄ and L₅ are independently a bond (single, double, or triple), a substituted or unsubstituted alkyl, or a carbonyl;
(b) A₄ and A₅ are independently a hydrogen, a substituted or unsubstituted alkyl, a substituted or unsubstituted aryl, a carbonyl, or an alkoxy;
(c) R₉—R₁₆ are independently a hydrogen, a hydroxyl, a substituted or unsubstituted alkyl, a carbonyl, an alkoxy, a thiol, or an amino; and
(d) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

32. The method of paragraph 30, wherein the second reactant is the same as the third reactant having the structure of:

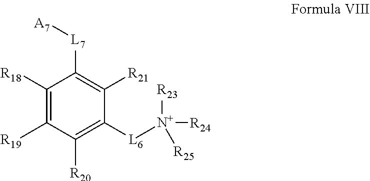

wherein:
(a) L₆ is a bond (single, double, or triple), a substituted or unsubstituted alkyl, a substituted or unsubstituted alkenyl, a carbonyl, or an alkoxy;
(b) L₇ is a bond (single, double, or triple), a substituted or unsubstituted alkyl, or a carbonyl;
(c) A₇ is a hydrogen, a substituted or unsubstituted alkyl, a substituted or unsubstituted aryl, a carbonyl, or an alkoxy;
(d) R₁₈—R₂₁ are independently a hydrogen, a hydroxyl, a substituted or unsubstituted alkyl, a carbonyl, an alkoxy, a thiol, or an amino;
(e) R₂₃—R₂₅ are independently a hydrogen or a substituted or unsubstituted alkyl; and
(f) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

33. The method of paragraph 32 further comprising (i-a) converting a pre-reactant having the structure of Formula VIII′ to the reactant having the structure of Formula VIII,

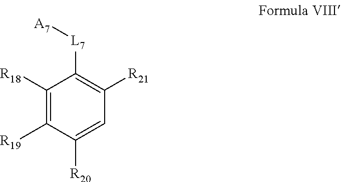

wherein:
(a) L₇ is a bond (single, double, or triple), a substituted or unsubstituted alkyl, or a carbonyl;
(b) A₇ is a hydrogen, a substituted or unsubstituted alkyl, a substituted or unsubstituted aryl, a carbonyl, or an alkoxy;
(c) R₁₈—R₂₁ are independently a hydrogen, a hydroxyl, a substituted or unsubstituted alkyl, a carbonyl, an alkoxy, a thiol, or an amino; and
(d) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

34. The method of paragraph 32 or 33, wherein R₁₉ is methyl; R₁₈ is —OR₂₂, each R₂₂ is hydrogen or a substituted or unsubstituted alkyl.

35. The method of paragraph 34 further comprising (i-b) converting a first starting material having the structure of Formula X to the pre-reactant having the structure of Formula VIII′, or (i-b) converting phloroglucinol to the pre-reactant having the structure of Formula VIII′:

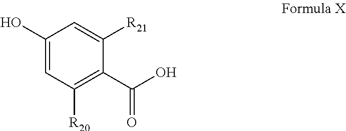

wherein (a) R₂₀ and R₂₁ are independently a hydrogen, a hydroxyl, a substituted or unsubstituted alkyl, a carbonyl, an alkoxy, a thiol, or an amino; and (b) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

36. The method of paragraph 35, wherein the method comprises (i-b) converting a first starting material having the structure of Formula X to the pre-reactant having the structure of Formula VIII′, and wherein step (i-b) comprises (i-b1) converting the first starting material having the structure of Formula X to a first intermediate having the structure of Formula IX and (i-b2) converting the first intermediate having the structure of Formula IX to the pre-reactant having the structure of Formula VIII′:

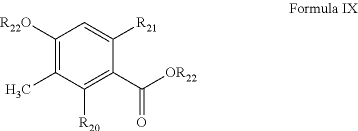

wherein (a) R₂₀ and R₂₁ are independently a hydrogen, a hydroxyl, a substituted or unsubstituted alkyl, a carbonyl, an alkoxy, a thiol, or an amino; (b) R₂₂ is a hydrogen or a substituted or unsubstituted alkyl; and (c) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

37. The method of paragraph 35, wherein the method comprises (i-b) converting phloroglucinol to the pre-reactant having the structure of Formula VIII′, and wherein step (i-b) comprises (i-b1′) converting phloroglucinol to a second intermediate having the structure of Formula XVII and (i-b2′) converting the second intermediate having the structure of Formula XVII to the pre-reactant having the structure of Formula VIII′:

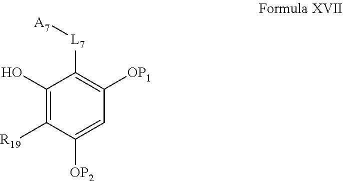

wherein: (a) L₇ is a bond (single, double, or triple), a substituted or unsubstituted alkyl, or a carbonyl; (b) A₇ is a hydrogen, a substituted or unsubstituted alkyl, a substituted or unsubstituted aryl, a carbonyl, or an alkoxy; (c) R₁₉ is a hydrogen, a hydroxyl, a substituted or unsubstituted alkyl, a carbonyl, an alkoxy, a thiol, or an amino; (d) P₁ and P₂ are independently a protection group, such as a methoxymethyl ether, tetrahydropyranyl ether, butyl ether, allyl ether, acetonide, benzylidene acetal, acetic acid esters, pivalic acid ester, tert-butyldimethylsilyl ethers, tert-butyldibenzylsilyl ethers, benzyl ether, benzoic acid ester, benzoate ester, or benzoate, or a combination thereof; and (e) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

38. The method of any one of paragraphs 30-37, wherein:
the first reactant is

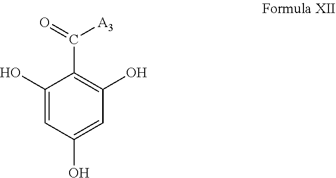

and the second reactant is the same as the third reactant and has the structure of:

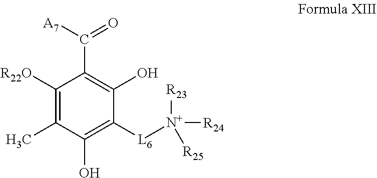

wherein:
(a) A₃ and A₇ are independently a hydrogen, a substituted or unsubstituted alkyl, a substituted or unsubstituted aryl, a carbonyl, or an alkoxy, optionally A₃ and A₇ are independently a substituted or unsubstituted alkyl, such as an unsubstituted C₁-C₁₀ alkyl, an unsubstituted C₁-C₈ alkyl, an unsubstituted C₁-C₇ alkyl, an unsubstituted C₁-C₆ alkyl, an unsubstituted C₁-C₅ alkyl, an unsubstituted C₁-C₄ alkyl, or an unsubstituted C₁-C₃ alkyl;
(b) L₆ is a bond (single, double, or triple), a substituted or unsubstituted alkyl, a substituted or unsubstituted alkenyl, a carbonyl, or an alkoxy;
(c) R₂₂ is a hydrogen or a substituted or unsubstituted alkyl;
(d) R₂₃—R₂₅ are independently a hydrogen or a substituted or unsubstituted alkyl; and
(e) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

39. The method of any one of paragraphs 30-38, wherein the solvent is dichloromethane, dimethyl sulfate, dimethyl sulfoxide, dimethyl formamide, butyryl chloride, dichloroethane, nitrobenzene, dioxane, 2-methylbutyryl chloride, ethyl acetate, ethyl lactate, acetone, 1-butanol, 1-propanol, 2-propanol, ethanol, isopropyl acetate, methanol, methyl ethyl ketone, t-butanol, tetrahydrofuran, 2-methyl tetrahydrofuran, acetonitrile, or toluene, or a combination thereof, or a mixture with water thereof.

40. The method of any one of paragraphs 30-39, wherein the reaction mixture is heated at a temperature of up to 130° C., in a range from about 100° C. to about 130° C., from about 100° C. to about 125° C., from about 100° C. to about 120° C., at 1 atm, such as about 110° C., for a time period in a range from about 30 mins to about 4 hours, from about 30 mins to about 3.5 hours, from about 30 mins to about 3 hours, from about 30 mins to about 2.5 hours, from about 1 hour to about 4 hours, from about 1 hour to about 3 hours, or from about 1 hour to about 2 hours, optionally under reflux.

41. The method of any one of paragraphs 30-40, wherein the compound has an overall yield of at least 7%, at least 10%, at least 15%, at least 20%, at least 25%, or at least 30%.

42. The method of any one of paragraphs 30-41 further comprising mixing the first reactant, the second reactant, the third reactant, and the solvent to form the reaction mixture prior to step (i) and/or purifying the product containing the prodrug subsequent to step (i).

43. A method for treating a cancer, reducing a cancer, or treating or ameliorating one or more symptoms associated with a cancer in a subject comprising:
(i) administering to the subject the pharmaceutical formulation of any one of paragraphs 22-29, wherein step (i) occurs one or more times.

44. The method of paragraph 43, wherein the method comprises only a single administration of the pharmaceutical formulation, wherein the pharmaceutical formulation comprises an effective amount of the compounds to inhibit the growth of a tumor by at least 40%, at least 50%, or at least 55% in the subject compared to the same tumor in a control subject, or
wherein the method comprises more than one step of administering to the subject the pharmaceutical formulation, wherein following all of the administration steps an effective amount of the compounds to inhibit the growth of a tumor by at least 40%, at least 50%, or at least 55% in the subject compared to the same tumor in a control subject is administered to the subject.

45. The method of paragraph 44, wherein the effective amount of the compounds is in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 3 mg/kg.

46. The method of any one of paragraphs 43-45, wherein the subject is a mammal.

47. The method of any one of paragraphs 43-46, wherein the pharmaceutical formulation is administered by oral administration, parenteral administration, inhalation, mucosal administration, topical or a combination thereof.

48. The method of any one of paragraphs 43-47, wherein the cancer is non-small-cell lung cancer.

49. The method of any one of paragraphs 43-48 further comprising administering to the subject a second active agent, optionally more than one second active agent, prior to, during, and/or subsequent to step (i).

50. The method of paragraph 49, wherein the second active agent is an anticancer agent.

51. A method for treating cancer cells and/or cancer stem cells in a subject in need thereof comprising (i) administering to the subject the pharmaceutical formulation of any one of paragraphs 22-29, wherein step (i) occurs one or more times.

52. The method of paragraph 51, wherein the compound has an IC₅₀ value against test cancer cells lower than IC₅₀ value of the same compound against non-cancerous cells, tested under the same conditions.

53. The method of paragraph 52, wherein the test cancer cells are MDA-MB-231, MCF-7, HepG2, Hela, AGS, HTC116, SW480, SUNE-1, H460, HCC827, H1650, or A549, or a combination thereof.

54. The method of paragraph 52 or paragraph 53, wherein the non-cancerous cells are CCD-19Lu.

55. The method of any one of paragraphs 51-54, wherein the method comprises only a single administration of the pharmaceutical formulation, wherein the pharmaceutical formulation comprises an effective amount of the compounds to induce mitochondria dysfunction and/or to inhibit ER to Golgi trafficking in cancer cells in the subject, compared to the subject before administered with the pharmaceutical formulation, or
wherein the method comprises more than one step of administering to the subject the pharmaceutical formulation, wherein following all of the administration steps an effective amount of the compounds to induce mitochondria dysfunction and/or to inhibit ER to Golgi trafficking in cancer cells in the subject, compared to the subject before administered with the pharmaceutical formulation is administered in the subject.

56. The method of any one of paragraphs 51-55, wherein the method comprises only a single administration of the pharmaceutical formulation, wherein the pharmaceutical formulation comprises an effective amount of the compounds to induce apoptosis of cancer cells in the subject, compared to the subject before administered with the pharmaceutical formulation, or
wherein the method comprises more than one step of administering to the subject the pharmaceutical formulation, wherein following all of the administration steps an effective amount of the compounds to induce apoptosis of cancer cells in the subject, compared to the subject before administered with the pharmaceutical formulation, is administered in the subject.

The present invention will be further understood by reference to the following non-limiting examples.

### EXAMPLES

### Example 1. Synthetic Agrimol B and its Derivatives Show Anticancer Activity

### Materials and Methods

### Synthesis of Agrimol B

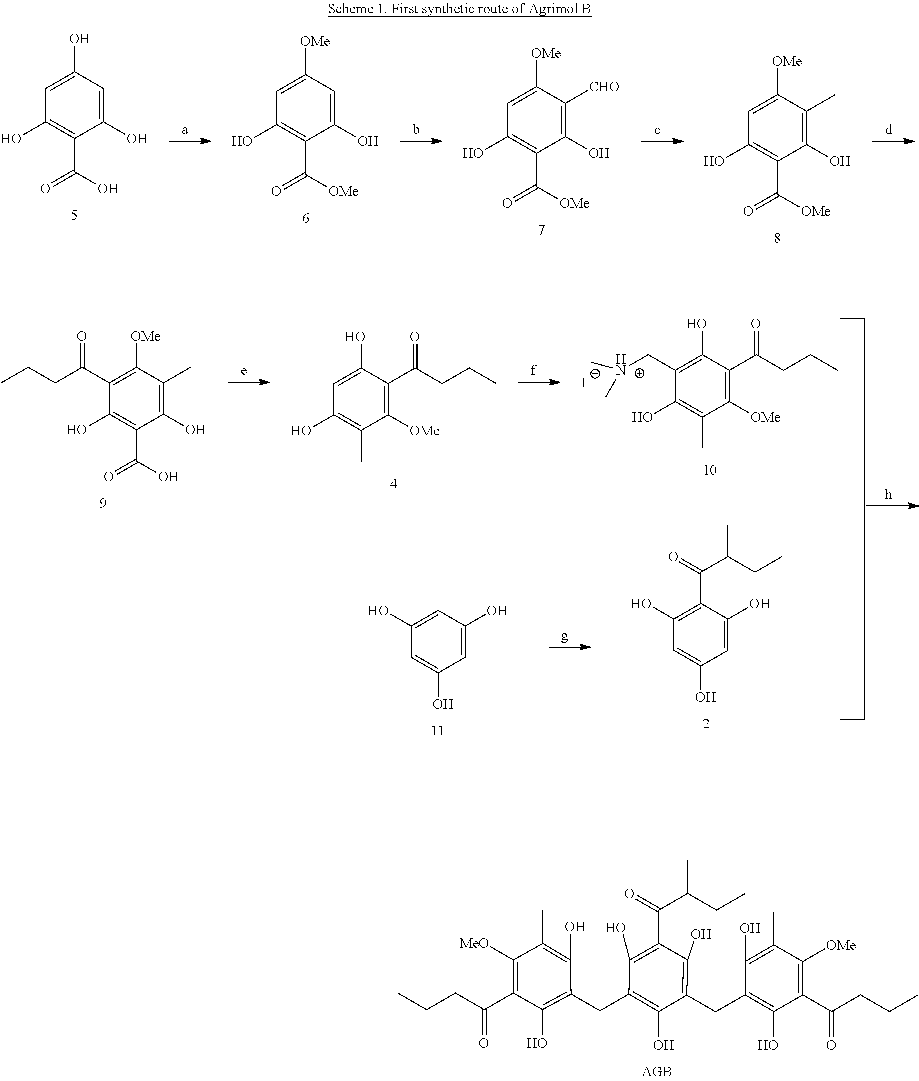

Reagents and conditions: (a) K₂CO₃, dimethyl sulfate, acetone, 0° C.-r.t., 18 hr, 87%; (b) DMF, POCl₃ cat., EtOAc, r.t., 3 hr, 92%; (c) NaBH₃CN, THF, r.t., 3 hr, 72%; (d) butyryl chloride, 1,2-dichloroethane/nitrobenzene, AlCl₃ cat., 80° C., 16 hr, 42%; (e) dioxane/H₂O (2:5), reflux, 5 hr, 85%; (f) Eschenmoser's salt, CHCl₃, r.t., 1.5 hr, 81%; (g) 2-methylbutyryl chloride, 1,2-dichloroethane/nitrobenzene, AlCl₃ cat., 80° C., 4 hr, 83%; (h) toluene, reflux, 2 hr, 53%. The overall yield is 7.3%, significantly higher than that polished (0.6%, Li et al. Acta Chimica Sinica, 1978, 36, 43-48; 0.3%, Zhu et al. Acta Pharmaceutica Sinica, 1989, 24, 431-437).

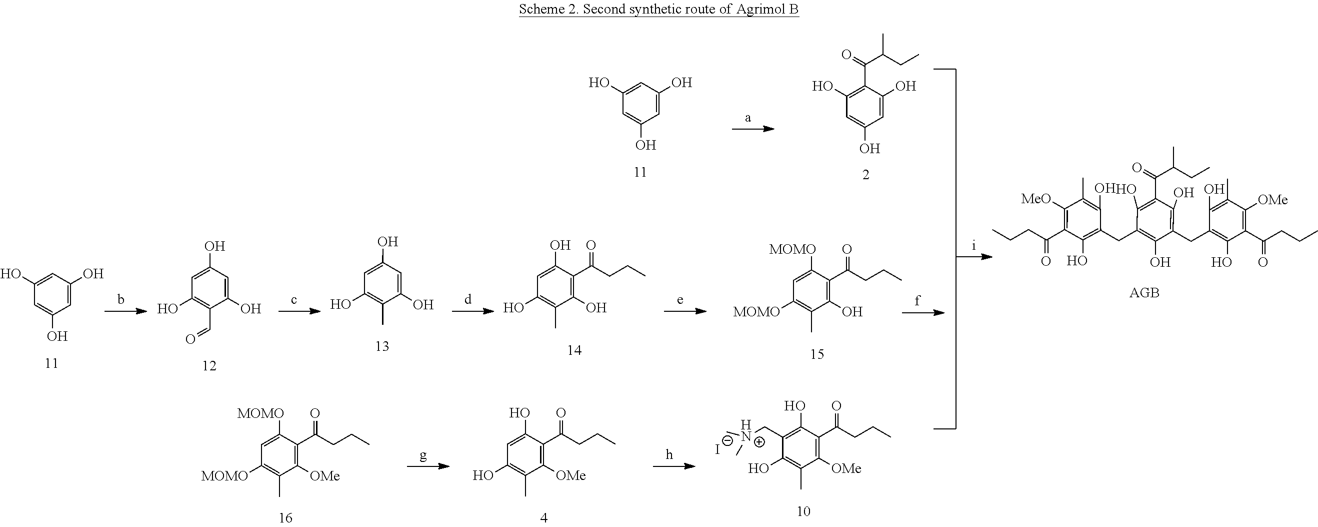

Reagents and conditions: (a) 2-methylbutyryl chloride, 1,2-dichloroethane/nitrobenze; AlCl₃ cat., 80° C., 3 h, 89%; (b) DMF, POCl₃ cat., EtOAc, r.t., 4 h, 89%; (c) NaBH₃CN, THF, r.t., pH<4, 4 h, 90%; (d) butyryl chloride, 1,2-dichloroethane/nitrobenzene AlCl₃ cat., 80° C., 3 h, 85%; (e) MOMCl, K₂CO₃, acetone, r.t., 1 h, 88%; (f) Dimethyl sulfate, K₂CO₃, acetone, reflux., overnight, 93%; (g) 1M HCl in methanol, reflux, 1 h, 95%; (h) Eschenmoser's salt, CHCl₃, r.t., 1.5 h, 81%; (i) toluene, reflux, 2 h, 53%. The overall yield is 20.2%.

All reactions were carried out with magnetic stirring, and air sensitive under argon atmosphere using standard Schlenk techniques in oven-dried glassware. External bath temperatures were used to record all reaction temperatures. All reagents with a purity >95% were obtained from commercial sources (Sigma Aldrich, J &K, Alfa Aesar and others) and used without further purification. Flash column chromatography was carried out with Merck silica gel 60 (0.040-0.063 mm). Analytical thin layer chromatography (TLC) was carried out using Merck silica gel 60 F254 μlass-backed plates.

NMR spectra (1H-NMR and 13C-NMR) were recorded in deuterated chloroform (CDCl3-d) or methanol (MeOD-d4) on a Bruker DRX 500 MHz spectrometer and reported as follows: chemical shift δ in ppm (multiplicity, coupling constant Jin Hz, number of protons) for 1H-NMR spectra and chemical shift δ in ppm for 13C-NMR spectra. Multiplicities are abbreviated as follows: s=singlet, d=doublet, t=triplet, q=quartet, m=multiplet, or combinations thereof. Residual solvent peaks of CDCl3 (δH=7.26 ppm, δC 77.16 ppm) and MeOD-d4 (δH 3.31 ppm, δC 49.00 ppm) were used as internal reference. High resolution mass spectra (HRMS) were recorded on a Bruker's Ultra-High-Resolution maXis II™ ESI-Q-TOF Mass Spectrometry.

### First Synthetic Route

### Methyl 2,6-dihydroxy-4-methoxybenzoate (6)

To a stirred suspension of 2,4,6-trihydroxybenzoic acid 5 (10.0 g, 53.2 mmol) in acetone (300 mL) was added potassium carbonate (14.6 g, 106.4 mmol) and dimethyl sulfate (10.0 mL, 106.4 mmol). The reaction mixture was stirred at room temperature for overnight and subsequently added 100 ml H₂O. The aqueous layer was extracted with ethyl acetate (EtOAc, 3×150 mL) and the combined ethyl acetate extracts were washed with brine and dried over anhydrous sodium sulfate and concentrated under reduced pressure. The title compound 6 (11.9 g, yield 87%) was afforded as a colorless solid by flash chromatography using hexanes/EtOAc 4:1. ¹H-NMR (500 MHz, chloroform-d): δ 6.03 (2H, s, 3&5-ArH), 4.02 (3H, s, 9-CH₃), 3.78 (3H, s, 7-CH₃). ¹³C-NMR (126 MHz, chloroform-d): δ 171.8 (8-C═O), 166.6 (4-ArC), 166.1 (6-ArC), 165.5 (2-ArC), 94.6 (3&5-ArC), 91.7 (1-ArC), 55.6 (7-CH₃), 52.6 (9-CH₃). HR-MS (negative mode): calculated for [M−H]⁻ 197.0455; Found 197.0458.

### Methyl 3-formyl-2,6-dihydroxy-4-methoxybenzoate (7)

To a solution of 6 (5.94 g, 30 mmol) in EtOAc (60 ml) was added dimethylformamide (DMF, 2.79 ml, 36 mmol) and phosphoryl chloride (POCl₃, 3.37 ml, 36 mmol) at 0° C. in an ice bath. Remove ice bath and the solution was stirred for 2 h. Water was added and the reaction mixture was refluxed for 30 min. When the solution cooled down, EtOAc was added to extract. The organic layers were combined and washed with brine, and dried over anhydrous Na₂SO₄. And then filtration and concentration in vacuo gave a residue, which was further purified by flash column chromatography on silica gel (hexane-EtOAc, 4:1) to yield 7 (6.21 g, 92%) as a light yellow solid. ¹H-NMR (500 MHz, chloroform-d): δ 10.01 (1H, s, 8-CHO), 5.95 (1H, s, 5-ArH), 4.03 (3H, s, 10-CH₃), 3.83 (3H, s, 7-CH₃). ¹³C-NMR (126 MHz, chloroform-d): δ 192.0 (8-C═O), 165.4 (9-C═O), 165.3 (4-ArC), 165.3 (2-ArC), 162.9 (6-ArC), 105.5 (3-ArC), 104.3 (1-ArC), 86.0 (5-ArC), 56.3 (7-CH₃), 52.4 (10-CH₃). HR-MS (negative mode): calculated for [M−H]⁻ 225.0405; Found 225.0400.

### Methyl 2,6-dihydroxy-4-methoxy-3-methylbenzoate (8)

To a solution of 7 (4.50 g, 20 mmol) in THF (100 ml) was added sodium cyanoborohydride (3.81 g, 60 mmol) and a drop of methyl orange was used as PH indicator at 0° C. in an ice bath, and 1N hydrochloric acid was added to keep the reaction mixture red (acidic environment). Remove ice bath and the solution was stirred until consumption of the starting material (around 2-3 h). THF was removed under reduced pressure to give the residue, which was extracted with ethyl acetate and dried over anhydrous Na₂SO₄, and then filtration and concentration in vacuo gave a residue, which was further purified by flash column chromatography on silica gel (hexane-EtOAc, 2:1) to yield the compound 8 (3.05 g, 72%) as a white solid. ¹H-NMR (500 MHz, chloroform-d): δ 6.06 (1H, s, 5-ArH), 4.03 (3H, s, 10-CH₃), 3.83 (3H, s, 7-CH₃), 1.99 (3H, s, 8-CH₃). ¹³C-NMR (126 MHz, chloroform-d): δ 170.1 (9-C═O), 164.4 (2,4&6-ArC), 104.9 (3-ArC), 93.6 (1-ArC), 91.5 (5-ArC), 55.8 (7-CH₃), 52.6 (10-CH₃), 7.5 (8-CH₃). HR-MS (negative mode): calculated for [M−H]⁻ 211.0612; Found 211.0608.

### 3-butyryl-2,6-dihydroxy-4-methoxy-5-methylbenzoic acid (9)

A solution of compound 8 (2.12 g, 10 mmol) and anhydrous aluminium chloride (5.33 g, 40 mmol) in 1,2-dichloroethane and nitrobenzene (1:1, 50 ml) under an argon atmosphere protection was stirred at ice bath for 20 min. Butyryl chloride (1.55 ml, 15 mmol) in 10 ml nitrobenzene was added and remove the ice bath to allow the reaction mixture was continued to stir at room temperature for 30 min. Then the reaction temperature was increased to 80° C. and kept for overnight. After cooling, the reaction mixture was diluted with ethyl acetate. Diluted HCl solution was added to the resulting mixture, which led to the formation of a white precipitate in the aqueous layer. The organic layer was decanted and the remaining solid residue was washed three times with EtOAc. The combined EtOAc layer was evaporated in vacuo and the remaining viscous oil was purified by silica gel column chromatography using n-hexane-EtOAc (10:1) firstly to remove the nitrobenzene and change eluent to dichloromethane-methnol (5:1) to obtain the 9 (1.12 g, 42%). ¹H-NMR (500 MHz, methanol-d₄): δ 3.78 (3H, s, 11-CH₃), 2.94-2.96 (2H, t, J=7.5 Hz, 8-CH₂), 2.06 (3H, s, 12-CH₃), 1.66-1.72 (2H, q, J=7.6 Hz, 9-CH₂), 0.97-1.00 (3H, t, J=7.5 Hz, 10-CH₃). ¹³C-NMR (126 MHz, methanol-d₄): δ 206.2 (7-C═O), 171.8 (13-C═O), 165.3 (6-ArC), 163.7 (4-ArC), 161.4 (2-ArC), 111.4 (5-ArC), 110.6 (3-ArC), 96.8 (1-ArC), 61.2 (11-CH₃), 45.4 (8-CH₂), 17.5 (9-CH₂), 12.8 (10-CH₃), 7.4 (12-CH₃). HR-MS (negative mode): calculated for [M−H]⁻ 267.0874; Found 267.0870.

### 1-(4,6-dihydroxy-2-methoxy-3-methylphenyl) butan-1-one (4)

A suspension of 9 (0.80 g, 3 mmol) in dioxane and H₂O (2:5, 28 ml) was refluxed for 5 h under argon atmosphere protection. After cooling to room temperature, the dioxane was removed by rotary evaporator and the solution was extracted three times with EtOAc (10 ml×3). The combined extracts were washed with brine and dried over anhydrous Na₂SO₄. The solvent was removed in vacuo and the crude product purified by flash silica chromatography (Hexane:EtOAc=3:1) to give 0.57 g (85%) of title compound 4. ¹H-NMR (500 MHz, chloroform-d): δ 6.19 (1H, s, 5-ArH), 3.74 (3H, s, 11-CH₃), 3.03-3.06 (2H, t, J=7.5 Hz, 8-CH₂), 2.10 (3H, s, 12-CH₃), 1.69-1.75 (2H, q, J=7.6 Hz, 9-CH₂), 0.96-0.99 (3H, t, J=7.6 Hz, 10-CH₃). ¹³C-NMR (126 MHz, chloroform-d): δ 206.6 (7-C═O), 163.4 (4-ArC), 161.8 (6-ArC), 161.2 (2-ArC), 109.9 (3-ArC), 109.5 (1-ArC), 99.8 (5-ArC), 61.9 (11-CH₃), 44.7 (8-CH₂), 18.3 (9-CH₂), 14.1 (10-CH₃), 8.6 (12-CH₃). HR-MS (negative mode): calculated for [M−H]⁻ 223.0976; Found 223.0978.

### 1-(3-butyryl-2,6-dihydroxy-4-methoxy-5-methylphenyl)-N,N-dimethylmethanaminium iodide (10)

To a solution of 4 (224.1 mg, 1 mmol) in chloroform (5.0 ml) was added Eschenmoser's salt (555.1 mg, 3 mmol) under an argon atmosphere protection. After stirring for 30 min at room temperature, another Eschenmoser's salt (92.5 mg, 0.5 mmol) was added and continued to stir for 1 h. Five drops of H₂O were then added to the reaction mixture and it was evaporated. The obtained crude product was grinded in MTBE (methyl tert-butyl ether, containing 10% of dichloromethane), the insoluble substance was obtained by filtration and recovered in MeOH. The resulting solution was evaporated in vacuo to yield the compound 10 (330 mg, 80.7%) as a saffron yellow solid. The product could be further used without any purification. ¹H-NMR (500 MHz, chloroform-d): 4.26 (2H, d, J=5.8 Hz, 13-CH₂), 3.76 (3H, s, 11-CH₃), 3.07 (2H, t, J=7.3 Hz, 8-CH₂), 2.86 (δH, d, J=5.0 Hz, 14&15-CH₃), 2.18 (3H, s, 12-CH₃), 1.72 (2H, q, J=7.3 Hz, 9-CH₂), 0.98 (3H, t, J=7.4 Hz, 10-CH₃). ¹³C-NMR (126 MHz, chloroform-d): δ 207.0 (7-C═O), 163.3 (6-ArC), 163.1 (2-ArC), 163.0 (4-ArC), 110.1 (5-ArC), 109.1 (1-ArC), 102.4 (3-ArC), 61.7 (11-CH₃), 50.8 (13-CH₂), 44.9 (2C, 15&16-CH₃), 42.5 (8-CH₂), 18.2 (9-CH₂), 14.0 (10-CH₃), 9.8 (12-CH₃).

### 2-methyl-1-(2,4,6-trihydroxyphenyl) butan-1-one (2)

A solution of phloroglucinol 11 (0.63 g, 5 mmol) and anhydrous aluminium chloride (AlCl₃, 2.67 g, 20 mmol) in 1,2-dichloroethane and nitrobenzene (1:1, 20 ml) under an argon atmosphere protection was stirred at room temperature for 15 min. 2-Methylbutyryl chloride (0.93 ml, 7.5 mmol) in 5 ml nitrobenzene was added and the reaction temperature was increased to 80° C. and kept for 4 h. After cooling, the reaction mixture was diluted with ethyl acetate and further washed with 1N HCl solution. The organic layer was decanted and the remaining solid residue was washed three times with EtOAc (20 ml×3). The combined EtOAc extracts were washed with brine and dried over anhydrous Na₂SO₄, evaporated in vacuo and the remaining residue was purified by silica gel column chromatography using n-hexane-EtOAc (2:1) as eluent to obtain the compound 2 (0.87 g, yield 83%). ¹H-NMR (500 MHz, methanol-d₄): δ 5.80 (2H, s, 3&5-ArH), 3.91-3.78 (1H, m, 8-CH), 1.81 (1H, dqd, J=13.8, 7.4, 6.3 Hz, 9-CH₂-1′), 1.44-1.30 (1H, dqd, J=13.8, 7.4, 6.3 Hz, 9-CH₂-2′), 1.11 (3H, d, J=6.7 Hz, 11-CH₃), 0.90 (3H, t, J=7.4 Hz, 10-CH₃). ¹³C-NMR (126 MHz, methanol-d₄): δ 211.42 (7-C═O), 165.78 (4-ArC), 165.71 (2C, 2&6-ArC), 105.16 (1-ArC), 95.84 (2C, 3&5-ArC), 46.61 (8-CH), 28.04 (9-CH₂), 17.08 (11-CH₃), 12.31 (10-CH₃). HR-MS (negative mode): calculated for [M−H]⁻ 209.0819; Found 209.0817.

### Agrimol B

In this study, the Eschenmoser's salt was used for the methylene bridge formation between the side rings and the middle ring of agrimol B. To a solution of compound 2 (middle ring, 21 mg, 0.1 mmol) in toluene (5 ml) was added compound 10 (Eschenmoser's salt of side rings, 81.8 mg, 0.2 mmol) under an argon atmosphere protection. After a reflux for 2 h in oil bath, the reaction mixture was diluted with 20 ml dichloromethane and washed with 1N HCl solution. The obtained aqueous layer was extracted with dichloromethane three times (10 ml×3). The combined dichloromethane extracts were washed with brine and dried over anhydrous Na₂SO₄, filtered and evaporated. The crude residue was purified by flash silicon chromatography (n-hexane-EtOAc=4:1) to afford the final product of agrimol B (36.2 mg, 53.1%) as a light-yellow powder. ¹H-NMR (500 MHz, chloroform-d): δ 15.97-15.62 (2H, br, ArOH), 10.72 (1H, br, ArOH), 9.70-9.27 (4H, br, ArOH), 3.91 (1H, m, 8-CH), 3.82 (4H, s, 13′-CH₃), 3.72 (δH, s, 11′-CH₃), 3.09 (4H, t, J=7.3 Hz, 8′-CH₂), 2.11 (δH, s, 12′-CH₃), 1.80 (1H, m, 9-CH₂), 1.74 (4H, q, J=7.3 Hz, 9′-CH₂), 1.40 (1H, m, 9-CH₂), 1.15 (3H, d, J=6.8 Hz, 11-CH₃), 0.99 (δH, t, J=7.4 Hz, 10′-CH₃), 0.91 (3H, t, J=7.4 Hz, 10-CH₃). ¹³C-NMR (126 MHz, chloroform-d): δ 212.38 (7′-C═O), 207.24 (7-C═O), 162.13 (ArC), 161.81 (ArC), 161.32 (ArC), 160.41 (ArC), 159.80 (ArC), 159.67 (ArC), 159.57 (ArC), 158.56 (ArC), 158.06 (ArC), 112.73 (ArC), 112.43 (ArC), 109.75 (ArC), 109.37 (ArC), 107.94 (ArC), 106.21 (ArC), 105.80 (ArC), 105.45 (ArC), 61.71 (11′-CH₃), 46.08 (8-CH), 44.36 (8′-CH₂), 26.99 (9-CH₂), 18.37 (9′-CH₂), 16.84 (13′-CH₂), 16.64 (11-CH₃), 14.08 (10′-CH₃), 12.08 (10-CH₃), 9.32 (12′-CH₃). HR-MS (negative mode): calculated for [M−H]⁻ 681.2917; Found 681.2911.

### Second Synthetic Route

### 2,4,6-trihydroxybenzaldehyde (12)

To a solution of 11 (5 g, 39.7 mmol) in EtOAc (60 mL) was added dimethylformamide (DMF, 3.8 mL, 50 mmol) and phosphoryl chloride (POCl3, 4.5 mL, 50 mmol) at 0° C. in an ice bath and stirred for 10 min. Remove ice bath and the solution continue to stir for 4 h at room temperature. Then 60 mL of H2O was added and the reaction mixture was refluxed for 30 min. After cooling down, 50 mL of ethyl acetate was used to extract for three times. The organic layers were combined and washed with brine, and dried over anhydrous Na2SO4. And then filtration and concentration in vacuo gave a residue, which was purified by flash column chromatography on silica gel (n-hexane:EA=5:1) to yield compound 12 (5.4 g, 89%) as a white solid. ¹H-NMR (500 MHz, methanol-d₄): δ 10.01 (s, 1H), 5.78 (s, 2H). ¹³C-NMR (126 MHz, methanol-d₄): δ 192.61, 168.78, 165.84, 106.24, 95.12. HR-MS (negative mode): calculated for [M−H]⁻ 153.0193; Found 153.0191.

### 2-methylbenzene-1,3,5-triol (13)

To a solution of 12 (5 g, 32.5 mmol) in THF (30 mL), sodium cyanoborohydride (6.1 g, 97.5 mmol) and methyl orange (4 drops) were added. The hydrochloric acid (1M) was used to adjust the reaction mixture solution to turn red color (pH<4). After stirring for 4 h at room temperature, the mixture was diluted with EtOAc, washed with brine and the layers were separated. The aqueous layer was extracted with EtOAc (30 mL) for three times, the combined organic layers were dried over Na₂SO₄, filtered and concentrated under reduced pressure. The obtained residue was purified by flash chromatography on silica gel (n-hexane:EA=3:1) to afford compound 13 (4.1 g, 90%) as alight beige solid. ¹H-NMR (500 MHz, methanol-d₄): δ 5.85 (s, 2H), 1.93 (s, 3H). ¹³C-NMR (126 MHz, methanol-d₄): δ 160.09, 157.64, 156.65, 103.53, 95.48, 95.39, 7.93. HR-MS (negative mode): calculated for [M−H]⁻ 139.0401; Found 139.0398.

### 1-(2,4,6-trihydroxy-3-methylphenyl) butan-1-one (14)

2-methylbenzene-1,3,5-triol (13, 2.0 g, 14.3 mmol) in nitrobenzene-1,2-dichloroethane mixed solution (1:1, 50 mL) was added into a two-necked round-bottomed flask and allowed to stir in an ice-bath while aluminum chloride (5.7 g, 43 mmol) was added. After 10-min stirring, a solution of butyryl chloride (1.8 mL, 17.1 mmol) was added dropwise to the reaction mixture and then the ice-batch was removed and the solution was refluxed for 2 h. After cooing to room temperature, the reaction mixture was then poured into a diluted HCl solution (1M, 100 mL) and the mixture extracted with 15 mL of ethyl acetate for three times. The organic solvents were combined and wash with brine followed a drying using anhydrous Na₂SO₄. The ethyl acetate was removed under reduced pressure. The oily residue containing the acylphloroglucinol was further purified by flash column chromatography on silica gel (n-hexane/EA=1:1) to yield 14 (2.5 g, 85%) as a light reddish brown solid. ¹H-NMR (500 MHz, methanol-d₄): δ 5.90 (s, 1H), 3.12-2.90 (m, 2H), 1.92 (s, 3H), 1.68 (h, J=7.5 Hz, 2H), 0.97 (t, J=7.4 Hz, 3H). ¹³C-NMR (126 MHz, methanol-d₄): δ 206.110, 163.64, 162.44, 160.06, 103.91, 102.28, 93.50, 45.58, 18.34, 13.12, 6.03. HR-MS (negative mode): calculated for [M−H]⁻ 209.0819; Found 209.0811.

### 1-(2-hydroxy-4,6-bis(methoxymethoxy)-3-methylphenyl) butan-1-one (15)

To a solution of 14 (2.1 g, 10 mmol) in dry acetone (10 mL), potassium carbonate (4.1 g, 30 mmol) was added the reaction mixture and stirred for 5 min at room temperature followed by the addition of chloromethyl methyl ether (MOMCl, 1.7 mL, 22 mmol). The reaction mixture was stirred for 1 h at room temperature. Acetone was removed from the reaction mixture under reduced pressure and the residue taken up in ethyl acetate (100 mL) and washed with brine (100 mL). The organic layer was dried using anhydrous Na2SO4, filtered and the ethyl acetate was removed under reduced pressure. The crude product was purified by flash column chromatography on silica gel using n-hexane and EtOAc (10:1) to obtain the compound 15 as a light-yellow powder (2.6 g, 88%). ¹H-NMR (400 MHz, chloroform-d): δ 13.89 (s, 1H), 6.39 (s, 1H), 5.24 (s, 2H), 5.22 (s, 2H), 3.51 (s, 3H), 3.48 (s, 3H), 3.04-2.97 (m, 2H), 2.05 (s, 3H), 1.77-1.66 (m, 2H), 0.98 (t, J=7.4 Hz, 3H). ¹³C-NMR (101 MHz, chloroform-d): δ 206.45, 163.84, 160.81, 158.42, 108.03, 106.76, 94.84, 94.22, 91.52, 56.79, 56.47, 46.53, 18.39, 14.14, 7.59. HR-MS (negative mode): calculated for [M−H]⁻ 297.1344; Found 297.1339.

### 1-(2-methoxy-4,6-bis(methoxymethoxy)-3-methylphenyl) butan-1-one (16)

To a solution of 15 (2.0 g, 6.7 mmol) in dry acetone (10 mL), potassium carbonate (2.8 g, 20 mmol) was added the reaction mixture and stirred for 5 min at room temperature followed by the addition of dimethyl sulfate (1.0 mL, 10 mmol). The reaction mixture was then heated to reflux for 4 h. After cooling down to room temperature, 50 mL of H₂O was added to the reaction mixture followed extracted using 50 mL of ethyl acetate three times. The organic layer was combined and wash by brine and dried using anhydrous Na₂SO₄. The obtained crude product after the removal of ethyl acetate under reduced pressure was further purified by flash column chromatography on silica gel using n-hexane and ethyl acetate (8:1) to yield compound 16 (1.9 g, 93%) as a colorless oily matter. ¹H-NMR (500 MHz, chloroform-d): δ 6.68 (s, 1H), 5.17 (s, 2H), 5.10 (s, 2H), 3.69 (s, 3H), 3.47 (s, 3H), 3.44 (s, 3H), 2.74 (t, J=7.3 Hz, 2H), 2.09 (s, 3H), 1.69 (q, J=7.3 Hz, 2H), 0.96 (t, J=7.4 Hz, 3H). ¹³C-NMR (126 MHz, chloroform-d): δ 205.05, 157.31, 156.14, 152.54, 121.06, 114.56, 98.11, 95.14, 94.77, 62.53, 56.42, 56.33, 47.16, 17.36, 8.70. HR-MS (positive mode): calculated for [M+H]⁺ 313.1646; Found 313.1655.

### 1-(4,6-dihydroxy-2-methoxy-3-methylphenyl) butan-1-one (4)

To a solution of 16 (1.57 g, 7 mmol) in methanol (5 mL), the concentrated HCl (0.5 mL) was added the reaction mixture and refluxed for 2 h. Methanol was removed from the reaction mixture under reduced pressure and the residue taken up in ethyl acetate (50 mL) and washed with brine (100 mL). The organic layer was dried using anhydrous Na2SO4, filtered and the ethyl acetate was removed under reduced pressure. The crude product was purified by flash column chromatography on silica gel using n-hexane and EtOAc (3:1) to get the compound 4 (1.5 g, 95%) as a white needle crystal. The spectroscopy data are the same as reported method.

Synthesis of compound 10 from compound 4, synthesis of compound 2, and synthesis of Agrimol B from compound 2 and compound 10 are as described above for synthetic route 1.

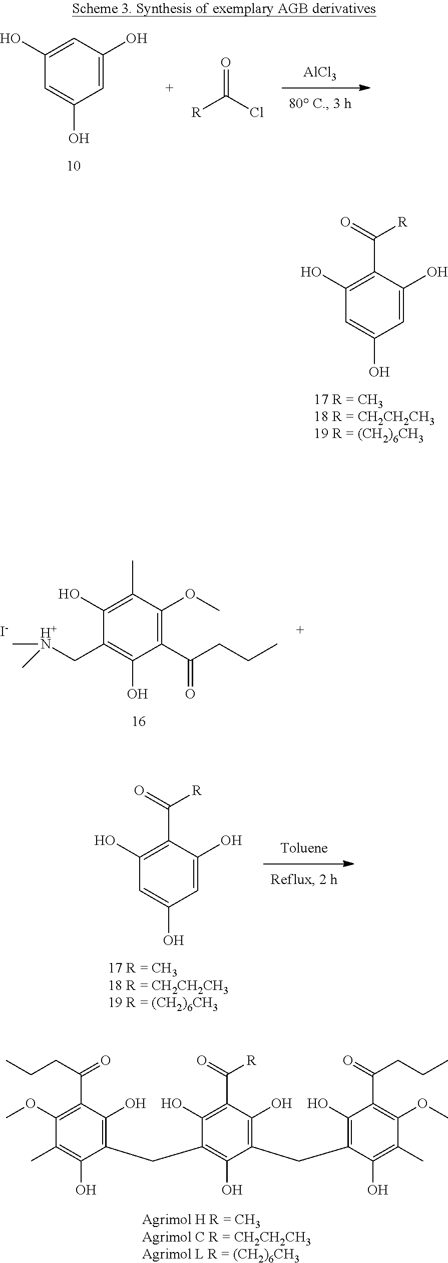

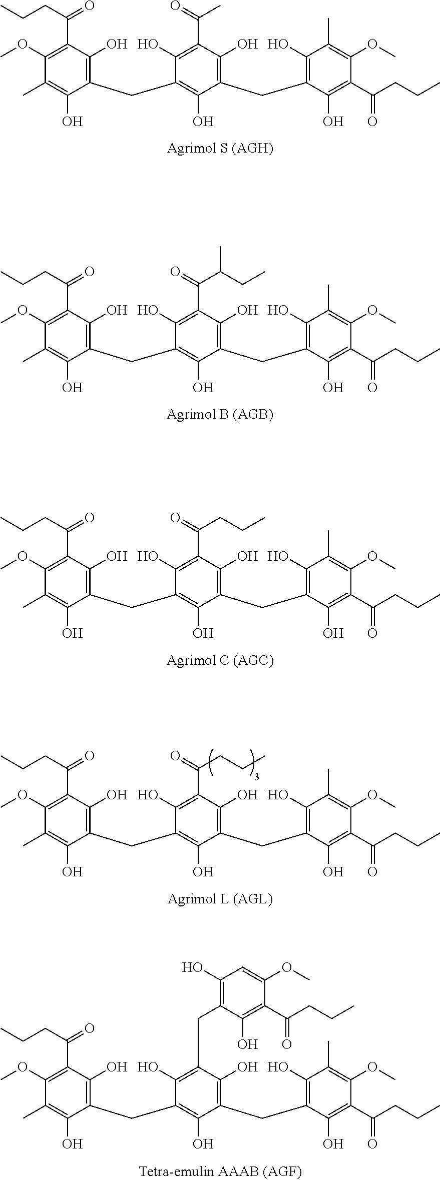

### 1-(2,4,6-trihydroxyphenyl) ethan-1-one (17)

A solution of phloroglucinol 10 (0.63 g, 5 mmol) and anhydrous aluminium chloride (AlCl₃, 2.67 g, 20 mmol) in 1,2-dichloroethane and nitrobenzene (1:1, 10 mL) under an argon atmosphere protection was stirred at room temperature for 15 min. Acetyl chloride (0.41 mL, 6 mmol) in 5 mL nitrobenzene was added and the reaction temperature was increased to 80° C. and kept for 4 h. After cooling, the reaction mixture was diluted with ethyl acetate and further washed with 1M HCl solution. The organic layer was decanted, and the remaining solid residue was washed three times with EtOAc (20 mL×3). The combined EtOAc extracts were washed with brine and dried over anhydrous Na₂SO₄, evaporated in vacuo and the residue was purified by silica gel column chromatography using n-hexane-EtOAc (2:1) as eluent to obtain the compound 17 (0.68 g, yield 81%). ¹H-NMR (500 MHz, methanol-d₄): δ 5.82 (2H, s), 2.61 (3H, s). 13C-NMR (126 MHz, methanol-d₄): δ 204.6, 166.3, 165.9, 105.6, 95.6, 32.7. HR-MS (negative mode): calculated for [M−H]⁻ 167.0350; Found 167.0357.

### 1-(2,4,6-trihydroxyphenyl) butan-1-one (18)

A solution of phloroglucinol 10 (0.63 g, 5 mmol) and anhydrous aluminium chloride (AiCl₃, 2.67 g, 20 mmol) in 1,2-dichloroethane and nitrobenzene (1:1, 10 mL) under an argon atmosphere protection was stirred at room temperature for 15 min. Butyryl chloride (0.64 mL, 6 mmol) in 5 mL nitrobenzene was added and the reaction temperature was increased to 80° C. and kept for 4 h. After cooling, the reaction mixture was diluted with ethyl acetate and further washed with 1M HCl solution. The organic layer was decanted, and the remaining solid residue was washed three times with EtOAc (20 mL×3). The combined EtOAc extracts were washed with brine and dried over anhydrous Na₂SO₄, evaporated in vacuo and the residue was purified by silica gel column chromatography using n-hexane-EtOAc (2:1) as eluent to obtain the compound 18 (0.88 g, yield 90.3%). ¹H-NMR (500 MHz, methanol-d₄): δ 5.80 (2H, s), 3.01 (t, J=7.4 Hz, 2H), 1.67 (q, J=7.4 Hz, 2H), 0.97 (t, J=7.4 Hz, 3H). ¹³C-NMR (126 MHz, methanol-d₄): δ 206.0, 164.7, 164.5, 110.1, 104.1, 94.4, 45.5, 18.2, 13.1. HR-MS (negative mode): calculated for [M−H]⁻ 195.0663; Found 195.0663.

### 1-(2,4,6-trihydroxyphenyl) octan-1-one (19)

A solution of phloroglucinol 10 (0.63 g, 5 mmol) and anhydrous aluminium chloride (AlCl₃, 2.67 g, 20 mmol) in 1,2-dichloroethane and nitrobenzene (1:1, 10 mL) under an argon atmosphere protection was stirred at room temperature for 15 min. Octanoyl chloride (1.03 mL, 6 mmol) in 3 mL nitrobenzene was added and the reaction temperature was increased to reflux and kept for 4 h. After cooling, the reaction mixture was diluted with ethyl acetate and further washed with 1M HCl solution. The organic layer was decanted, and the remaining solid residue was washed three times with EtOAc (20 mL×3). The combined EtOAc extracts were washed with brine and dried over anhydrous Na₂SO₄, evaporated in vacuo and the residue was purified by silica gel column chromatography using n-hexane-EtOAc (2:1) as eluent to obtain the compound 19 (1.16 g, yield 92%). ¹H-NMR (500 MHz, methanol-d₄): δ 5.80 (s, 2H), 3.07-2.94 (m, 2H), 1.65 (t, J=7.3 Hz, 2H), 1.41-1.22 (m, 8H), 0.96-0.84 (m, 3H). ¹³C-NMR (126 MHz, methanol-d₄): δ 207.52, 165.96, 165.80, 105.35, 95.71, 44.83, 32.95, 30.64, 30.31, 26.25, 23.69, 14.42. HR-MS (negative mode): calculated for [M−H]⁻ 251.1289; Found 251.1283.

### (S)-2-methyl-1-(2,4,6-trihydroxy-3-methylphenyl) butan-1-one (20)

A solution of 12 (0.7 g, 5 mmol) and anhydrous aluminium chloride (AlCl₃, 2.67 g, 20 mmol) in 1,2-dichloroethane and nitrobenzene (1:1, 10 mL) under an argon atmosphere protection was stirred at room temperature for 15 min. 2-methylbutyryl chloride (0.93 mL, 7.5 mmol) in 5 mL nitrobenzene was added and the reaction temperature was increased to 80° C. and kept for 4 h. After cooling, the reaction mixture was diluted with ethyl acetate and further washed with 1M HCl solution. The organic layer was decanted, and the remaining solid residue was washed three times with EtOAc (20 mL×3). The combined EtOAc extracts were washed with brine and dried over anhydrous Na₂SO₄, evaporated in vacuo and the remaining residue was purified by silica gel column chromatography using n-hexane-EtOAc (1:1) as eluent to obtain the compound 20 (0.85 g, yield 76%). ¹H-NMR (500 MHz, methanol-d₄): δ 5.89 (s, 1H), 3.88 (q, J=6.7 Hz, 1H), 1.92 (s, 3H), 1.80 (dt, J=13.8, 7.0 Hz, 1H), 1.36 (dt, J=13.9, 7.1 Hz, 1H), 1.11 (d, J=6.9 Hz, 3H), 0.89 (t, J=7.4 Hz, 3H). ¹³C-NMR (126 MHz, methanol-d₄): δ 210.32, 163.87, 162.31, 159.74, 103.84, 102.43, 95.59, 93.68, 45.40, 26.93, 15.96, 11.07, 6.10. HR-MS (negative mode): calculated for [M−H]⁻ 223.0976; Found 223.0973.

### Agrimol H

To a solution of compound 17 (21 mg, 0.12 mmol) in toluene (2 mL) was added compound 16 (98 mg, 0.24 mmol) under an argon atmosphere protection. After a reflux for 2 h in oil bath, the reaction mixture was diluted with 20 mL dichloromethane and washed with 1M HCl solution. The aqueous layer was extracted with dichloromethane three times (10 mL×3). The combined dichloromethane extracts were washed with brine and dried over anhydrous Na₂SO₄, filtered and evaporated. The crude residue was purified by flash silicon chromatography using dichloromethane to afford the product as a reddish-brown powder, which was a new compound and named Agrimol H (52.2 mg, 68%). ¹H-NMR (500 MHz, chloroform-d): δ 16.08-15.23 (m, 3H), 10.73 (s, 1H), 9.90-8.83 (m, 4H), 3.83 (d, J=8.6 Hz, 4H), 3.72 (s, 6H), 3.08 (q, J=7.9 Hz, 4H), 2.72 (s, 3H), 2.19-2.02 (m, 7H), 0.99 (q, J=7.3 Hz, 9H), 0.93-0.73 (m, 8H). ¹³C-NMR (126 MHz, chloroform-d): δ 205.50, 160.42, 158.37, 105.94, 77.36, 61.72, 44.32, 33.16, 32.07, 29.85, 29.51, 22.84, 18.35, 14.27, 14.09, 9.32. HR-MS (negative mode): calculated for [M−H]⁻ 639.2447; Found 639.2347.

### Agrimol C

To a solution of compound 18 (20 mg, 0.1 mmol) in toluene (2 mL) was added compound 16 (82 mg, 0.2 mmol) under an argon atmosphere protection. After a reflux for 3 h in oil bath, the reaction mixture was diluted with 20 mL dichloromethane and washed with 1M HCl solution. The aqueous layer was extracted with dichloromethane three times (10 mL×3). The combined dichloromethane extracts were washed with brine and dried over anhydrous Na₂SO₄, filtered and evaporated. The crude residue was purified by flash silicon chromatography using dichloromethane to afford the product of Agrimol C (56.8 mg, 85%) as a light-yellow powder. ¹H-NMR (500 MHz, chloroform-d): ¹H NMR (500 MHz, Chloroform-d) δ 15.48 (s, 2H), 9.43 (s, 4H), 3.80 (s, 4H), 3.71 (s, 6H), 3.06 (dt, J=10.8, 7.3 Hz, 6H), 2.10 (s, 6H), 1.72 (m, J=10.9, 7.4, 3.5, 3.1 Hz, 6H), 0.98 (td, J=7.4, 3.9 Hz, 9H). ¹³C-NMR (126 MHz, chloroform-d): δ 207.12, 206.72, 162.72, 161.78, 160.33, 159.63, 112.29, 110.13, 109.64, 108.04, 106.41, 104.14, 96.69, 61.70, 45.74, 44.42, 29.84, 18.36, 18.23, 16.01, 14.10, 14.07, 9.28. HR-MS (negative mode): calculated for [M−H]⁻ 639.2447; Found 639.2347.

### Agrimol L

To a solution of compound 19 (20 mg, 0.08 mmol) in toluene (2 mL) was added compound 16 (65.5 mg, 0.16 mmol) under an argon atmosphere protection. After a reflux for 4 h in oil bath, the reaction mixture was diluted with 20 mL dichloromethane and washed with 1M HCl solution. The obtained aqueous layer was extracted with dichloromethane three times (10 mL×3). The combined dichloromethane extracts were washed with brine and dried over anhydrous Na₂SO₄, filtered and evaporated. The crude residue was purified by flash silicon chromatography using dichloromethane to afford the product as a light-yellow powder, which was a new compound and named agrimol L (36.2 mg, 53.1%) as a light-yellow powder. ¹H-NMR (500 MHz, chloroform-d): δ 16.00 (br, 2H), 15.62 (s, 1H), 10.70 (s, 1H), 9.65 (d, J=4.4 Hz, 2H), 9.24 (s, 1H), 3.82 (s, 4H), 3.72 (s, 6H), 3.13 (t, J=7.4 Hz, 2H), 3.09 (t, J=7.3 Hz, 4H), 2.11 (s, 6H), 1.74 (q, J=7.3 Hz, 4H), 1.68-1.61 (m, 2H), 1.40-1.28 (m, 10H), 1.25 (s, 4H), 0.99 (t, J=7.4 Hz, 6H), 0.93-0.87 (m, 3H). ¹³C-NMR (126 MHz, chloroform-d): δ 208.37, 160.40, 158.03, 110.14, 107.95, 105.73, 61.72, 44.42, 31.90, 29.86, 29.53, 29.39, 24.78, 22.81, 18.37, 14.26, 14.09, 9.32. HR-MS (negative mode): calculated for [M−H]⁻ 723.3386; Found 723.3374.

### AGF

To a solution of compound 10 (20 mg, 0.16 mmol) in toluene (5 mL) was added compound 16 (204 mg, 0.5 mmol) under an argon atmosphere protection. After a reflux for 2 h in oil bath, the reaction mixture was diluted with 20 mL dichloromethane and washed with 1M HCl solution. The obtained aqueous layer was extracted with dichloromethane three times (10 mL×3). The combined dichloromethane extracts were washed with brine and dried over anhydrous Na2SO4, filtered and evaporated. The crude residue was purified by flash silicon chromatography using dichloromethane to afford the product of AGF (109 mg, 82%) as a light-yellow powder. 1H-NMR (500 MHz, chloroform-d): δ 15.78 (s, 3H), 10.01 (s, 3H), 9.60 (s, 3H), 3.84 (s, 6H), 3.70 (s, 9H), 3.08 (t, J 7.3 Hz, 6H), 2.09 (s, 9H), 1.74 (d, J 7.3 Hz, 6H), 0.98 (t, J 7.4 Hz, 9H). 13C-NMR (126 MHz, chloroform-d): δ 207.16, 162.01, 160.24, 159.60, 150.86, 112.54, 110.01, 107.89, 107.16, 61.71, 44.33, 29.84, 18.44, 17.62, 14.06, 9.30. HR-MS (negative mode): calculated for [M−H]− 833.3390; Found 833.3380.

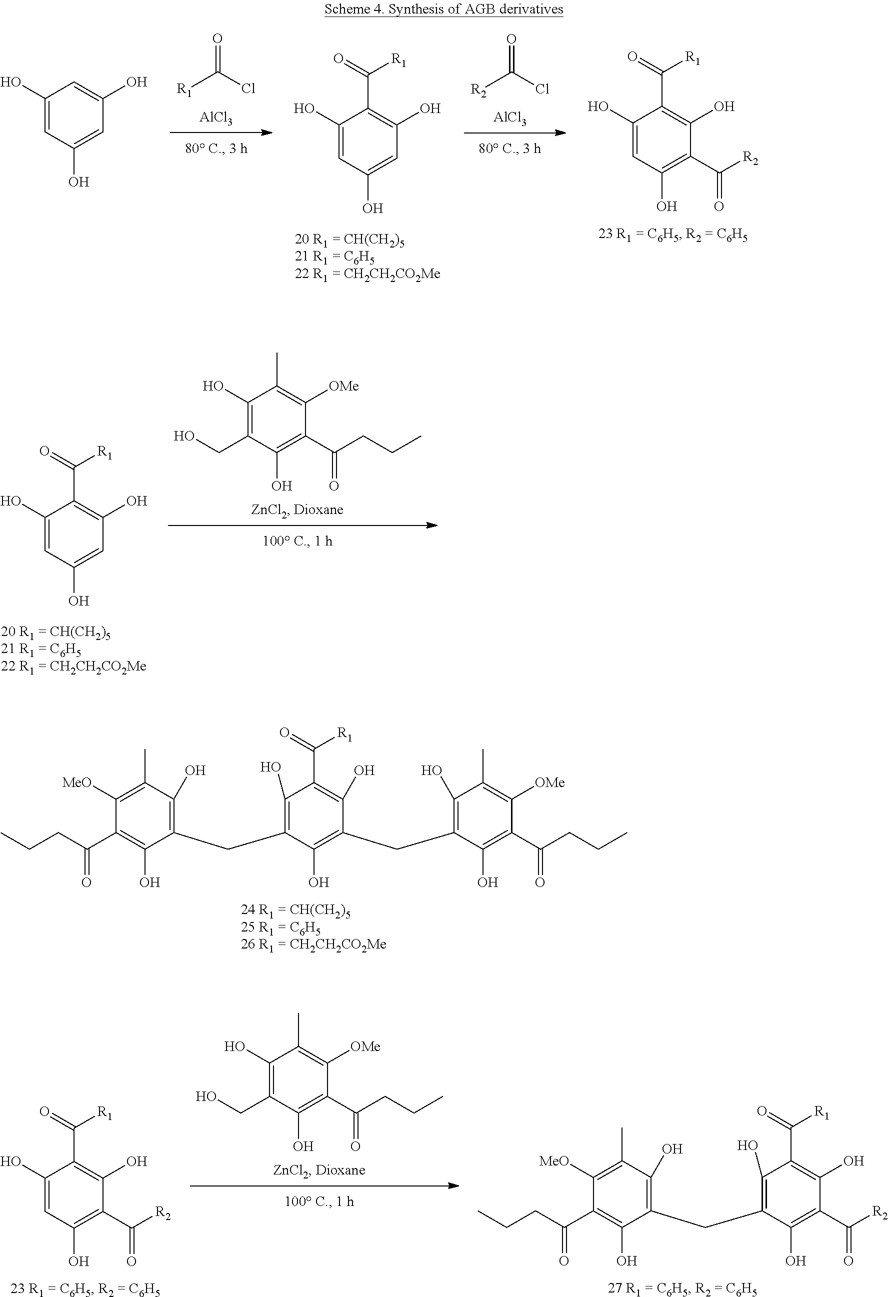

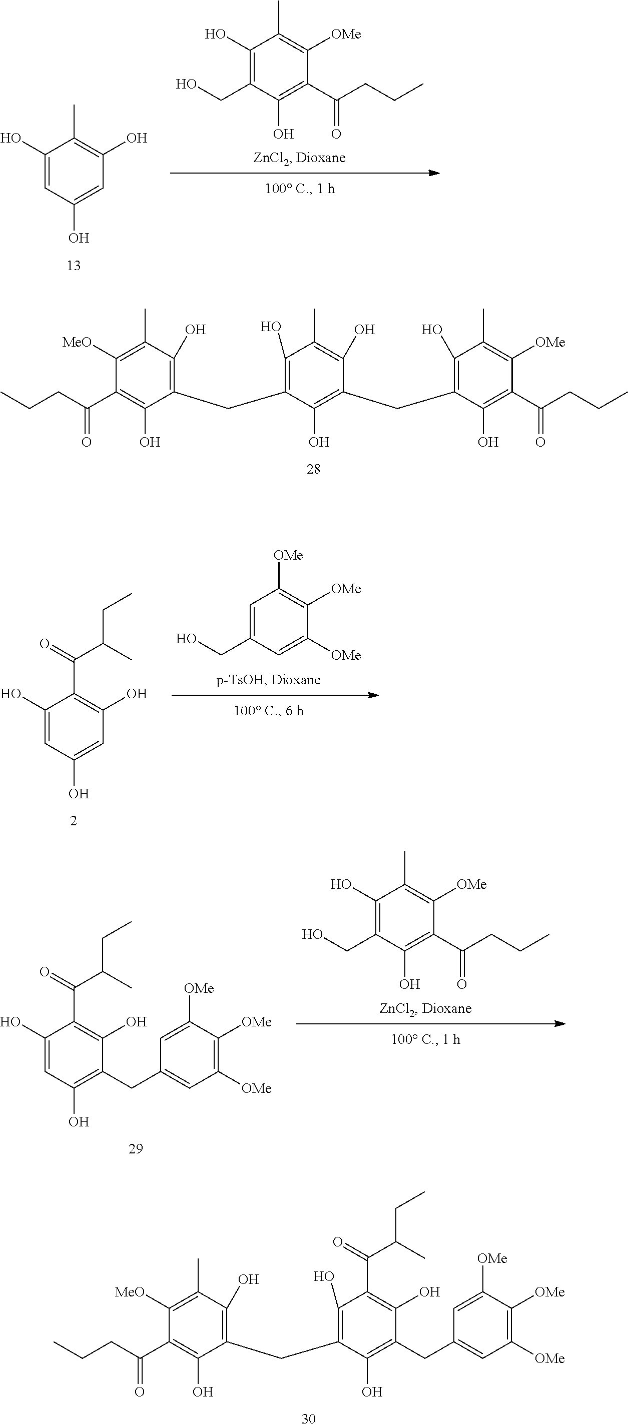

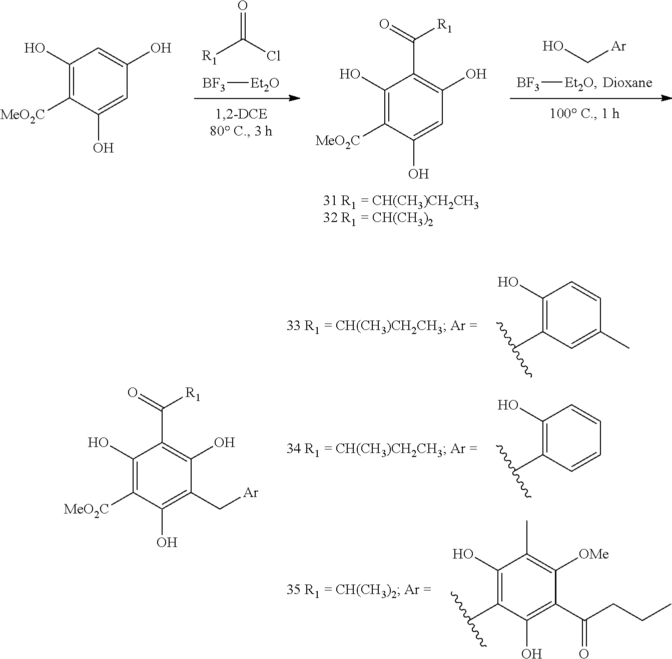

### General Procedure for Acrylation:

A solution of phloroglucinol 11 (1 eq.) and anhydrous aluminium chloride (4 eq.) in 1,2-dichloroethane and nitrobenzene (1:1, v/v, ˜1M) under an argon atmosphere protection was stirred at room temperature for 15 min. acryl chloride (1.5 eq.) in nitrobenzene was added and the reaction temperature was increased to 80° C. and kept for 4 h. After cooling, the reaction mixture was diluted with ethyl acetate and further washed with 1N HCl solution. The organic layer was decanted and the remaining solid residue was washed three times with EtOAc (20 ml×3). The combined EtOAc extracts were washed with brine and dried over anhydrous Na₂SO₄, evaporated in vacuo and the remaining residue was purified by silica gel column chromatography using n-hexane-EtOAc as eluent to obtain the compound.

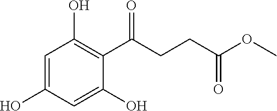 [SMILES: COC(=O)CCC(=O)c1c(O)cc(O)cc1O]

### Methyl 4-oxo-4-(2,4,6-trihydroxyphenyl)butanoate (22)

1.2 g, yield 75%. ¹H-NMR (600 MHz, methanol-d₄): δ 5.79 (s, 2H), 3.66 (s, 3H), 3.73 (t, J=6.6 Hz, 2H), 1.65 (t, J=6.6 Hz, 2H). ¹³C-NMR (150 MHz, methanol-d₄): δ 204.7, 176.1, 166.5, 166.1, 105.5, 95.9, 52.4, 40.0, 29.5. HR-MS (negative mode): calculated for [M−H]⁻ 239.0561; Found 239.0563.

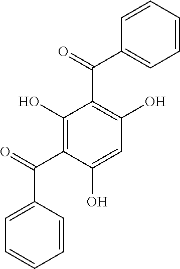 [SMILES: O=C(c1ccccc1)c1c(O)cc(O)c(C(=O)c2ccccc2)c1O]

### (2,4,6-trihydroxy-1,3-phenylene)bis(phenylmethanone) (23)

270 mg, yield 57%. ¹H-NMR (600 MHz, methanol-d₄): δ 7.71 (dt, J=7.0, 5.2 Hz, 4H), 7.75 (t, J=7.0 Hz, 2H), 7.47 (t, J=11.1 Hz, 4H), 6.02 (s, 1H). ¹³C-NMR (150 MHz, methanol-d₄): δ 200.0, 166.2, 142.0, 133.0, 129.7, 129.1, 106.4, 96.0. HR-MS (negative mode): calculated for [M−H]⁻ 333.0768; Found 333.0766.

### General Procedure for A-B Ring Coupling by ZnCl₂ (MethodA):

A solution of acryl compound (1 eq.) and benzyl alcohol (2 eq.) in dioxane (˜0.1M) under an argon atmosphere protection was stirred at room temperature. ZnCl₂ (2 eq.) was added and the reaction temperature was increased to 100° C. and kept for 1 h. After cooling, the reaction mixture was diluted with ethyl acetate and further washed with sat. NaHCO₃ solution. The mixture was washed three times with EtOAc. The combined EtOAc extracts were washed with brine and dried over anhydrous Na₂SO₄, evaporated in vacuo and the remaining residue was purified by silica gel column chromatography using n-hexane-EtOAc as eluent to obtain the compound.

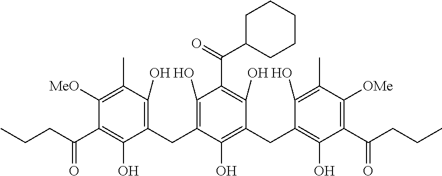 [SMILES: CCCC(=O)c1c(O)c(Cc2c(O)c(Cc3c(O)c(C)c(OC)c(C(=O)CCC)c3O)c(O)c(C(=O)C3CCCCC3)c2O)c(O)c(C)c1OC]

### 1,1′-(((5-(cyclohexanecarbonyl)-2,4,6-trihydroxy-1,3-phenylene)bis(methylene))bis(2,4-dihydroxy-6-methoxy-5-methyl-3,1-phenylene))bis(butan-1-one) (24)

Method A: 57 mg, yield 62%. ¹H-NMR (600 MHz, CDCl₃): δ 10.70 (s, 1H), 9.65 (brs, 2H), 9.28 (s, 1H), 3.81 (s, 4H), 3.71 (s, 6H), 3.08 (t, J=7.3 Hz, 4H), 2.11 (s, 6H), 1.89 (brs, 2H), 1.81 (brs, 2H), 1.74 (m, 5H), 1.39 (brs, 4H), 1.25 (m, 2H), 0.99 (t, J=7.3 Hz, 6H). ¹³C-NMR (150 MHz, CDCl₃): δ 211.4, 207.0, 161.9, 160.2, 159.2, 157.8, 112.5, 109.6, 107.8, 105.8, 104.9, 61.5, 49.6, 44.2, 29.9, 26.1, 18.1, 16.5, 13.9, 9.1. HR-MS (negative mode): calculated for [M−H]⁻ 707.3073; Found 707.3070.

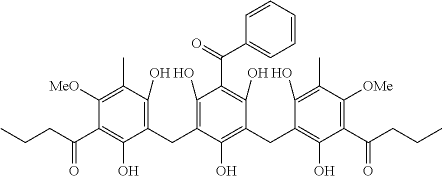 [SMILES: CCCC(=O)c1c(O)c(Cc2c(O)c(Cc3c(O)c(C)c(OC)c(C(=O)CCC)c3O)c(O)c(C(=O)c3ccccc3)c2O)c(O)c(C)c1OC]

### 1,1′-(((5-benzoyl-2,4,6-trihydroxy-1,3-phenylene)bis(methylene))bis(2,4-dihydroxy-6-methoxy-5-methyl-3,1-phenylene))bis(butan-1-one) (25)

Method A: 140 mg, yield 67%. ¹H-NMR (600 MHz, CDCl₃): δ 9.27 (s, 1H), 7.56 (d, J=7.2 Hz, 2H), 7.49 (m, 1H), 7.36 (m, 2H), 3.81 (s, 4H), 3.71 (s, 6H), 3.07 (t, J=7.2 Hz, 4H), 2.11 (s, 6H), 1.72 (q, J=7.3 Hz, 4H), 0.97 (d, J=7.3 Hz, 6H). ¹³C-NMR (150 MHz, CDCl₃): δ 207.0, 200.7, 161.6, 160.3, 159.5, 158.3, 141.0, 131.5, 128.2, 127.6, 112.4, 109.4, 107.8, 105.5, 61.6, 44.2, 18.2, 16.7, 13.9, 9.2. HR-MS (negative mode): calculated for [M−H]⁻ 701.2603; Found 701.2620.

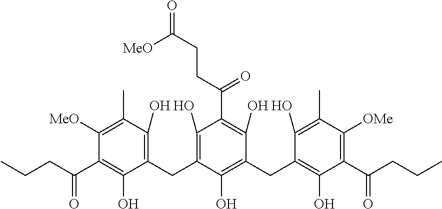 [SMILES: CCCC(=O)c1c(O)c(Cc2c(O)c(Cc3c(O)c(C)c(OC)c(C(=O)CCC)c3O)c(O)c(C(=O)CCC(=O)OC)c2O)c(O)c(C)c1OC]

### methyl 4-(3,5-bis(3-butyryl-2,6-dihydroxy-4-methoxy-5-methylbenzyl)-2,4,6-trihydroxyphenyl)-4-oxobutanoate (26)

Method A: 62 mg, yield 59%. ¹H-NMR (600 MHz, CDCl₃): δ 3.80 (s, 2H), 3.71 (s, δH), 3.70 (s, 3H), 3.50 (t, J=6.6 Hz, 2H), 3.09 (t, J=7.2 Hz, 4H), 2.67 (t, J=6.0 Hz, 2H), 2.10 (s, 6H), 1.73 (m, 4H), 0.99 (t, J=7.2 Hz, 6H). ¹³C-NMR (150 MHz, CDCl₃): δ 207.1, 200.7, 161.6, 160.2, 159.5, 158.3, 141.0, 131.5, 128.3, 127.6, 112.4, 109.4, 107.9, 105.5, 61.6, 44.2, 18.2, 16.7, 13.9, 9.2. HR-MS (negative mode): calculated for [M−H]⁻ 711.2658; Found 711.2662.

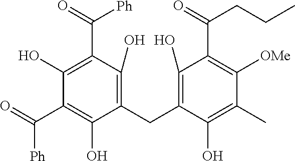 [SMILES: CCCC(=O)c1c(O)c(Cc2c(O)c(C(=O)c3ccccc3)c(O)c(C(=O)c3ccccc3)c2O)c(O)c(C)c1OC]

### (5-(3-butyryl-2,6-dihydroxy-4-methoxy-5-methylbenzyl)-2,4,6-trihydroxy-1,3 phenylene)bis(phenylmethanone) (27)

Method A: 141 mg, yield 72%. ¹H-NMR (600 MHz, CDCl₃): δ 15.55 (s, 1H), 13.27 (s, 1H), 9.04 (s, 1H), 7.60 (d, J=7.1 Hz, 4H), 7.52 (t, J=7.4 Hz, 2H), 7.41 (t, J=7.8 Hz, 4H), 3.81 (s, 2H), 3.82 (s, 3H), 3.72 (s, 3H), 3.06 (t, J=7.2 Hz, 2H), 2.13 (s, 3H), 1.71 (q, J=7.3 Hz, 2H), 0.96 (d, J=7.3 Hz, 3H). ¹³C-NMR (150 MHz, CDCl₃): δ 207.0, 200.1, 166.7, 165.2, 161.3, 160.3, 159.4, 140.6, 131.7, 128.1, 127.8, 112.4, 108.8, 107.9, 105.5, 104.1, 61.6, 44.2, 18.1, 16.0, 13.9, 9.2. HR-MS (negative mode): calculated for [M−H]—569.1817; Found 569.1810.

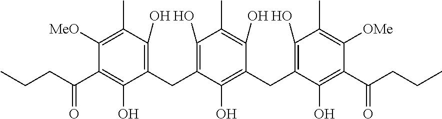 [SMILES: CCCC(=O)c1c(O)c(Cc2c(O)c(C)c(O)c(Cc3c(O)c(C)c(OC)c(C(=O)CCC)c3O)c2O)c(O)c(C)c1OC]

### 1,1′-(((2,4,6-trihydroxy-5-methyl-1,3-phenylene)bis(methylene))bis(2,4-dihydroxy-6-methoxy-5-methyl-3,1-phenylene))bis(butan-1-one) (28)

Method A: 105 mg, 86%. ¹H NMR (600 MHz, CDCl₃) δ 15.52 (s, 2H), 9.79 (s, 1H), 9.27 (s, 2H), 3.84 (s, 4H), 3.70 (s, 6H), 3.07 (t, J=7.3 Hz, 4H), 2.10 (m, 9H), 1.73 (h, J=7.4 Hz, 4H), 0.99 (t, J=7.4 Hz, 6H). ¹³C NMR (151 MHz, CDCl₃) δ 207.1, 161.5, 160.2, 159.7, 151.4, 149.8, 112.1, 110.1, 108.1, 106.0, 103.4, 61.7, 44.4, 18.4, 17.4, 14.1, 9.2, 8.5. HR-MS (negative mode): calculated for [M−H]⁻ 611.2497; Found 611.2512.

### General Procedure for A-B Ring Coupling Byp-TsOH (Method B):

A solution of acryl compound (1 eq.) and benzyl alcohol (1 eq.) in dioxane (˜0.1M) under an argon atmosphere protection was stirred at room temperature. p-TsOH (1 eq.) was added and the reaction temperature was increased to 100° C. and kept for 6 h. After cooling, the reaction mixture was diluted with ethyl acetate and further washed with sat. NaHCO₃ solution. The mixture was washed three times with EtOAc. The combined EtOAc extracts were washed with brine and dried over anhydrous Na₂SO₄, evaporated in vacuo and the remaining residue was purified by silica gel column chromatography using n-hexane-EtOAc as eluent to obtain the compound.

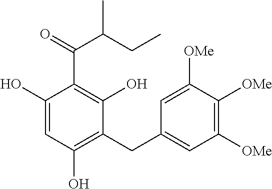 [SMILES: CCC(C)C(=O)c1c(O)cc(O)c(Cc2cc(OC)c(OC)c(OC)c2)c1O]

### 2-methyl-1-(2,4,6-trihydroxy-3-(3,4,5-trimethoxybenzyl)phenyl)butan-1-one (29)

Method B: 127 mg, yield 34%. ¹H-NMR (600 MHz, CDCl₃): δ 6.54 (s, 2H), 5.86 (s, 1H), 3.88 (s, 2H), 3.79 (s, 3H), 3.50 (m, 1H), 2.11 (s, 6H), 1.82 (m, 2H), 1.38 (m, 1H), 1.15 (d, J=6.72 Hz, 3H), 0.89 (t, J=7.3 Hz, 3H). ¹³C-NMR (150 MHz, CDCl₃): δ 210.8, 172.2, 164.0, 160.7, 159.4, 152.7, 137.1, 135.5, 106.7, 105.5, 104.5, 95.0, 60.8, 55.9, 45.8, 28.2, 26.9, 16.5, 11.9. HR-MS (negative mode): calculated for [M−H]⁻ 389.1605; Found 389.1600.

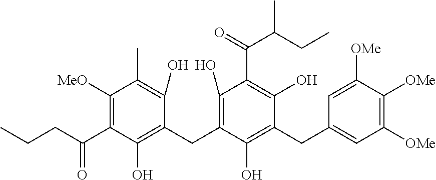 [SMILES: CCCC(=O)c1c(O)c(Cc2c(O)c(Cc3cc(OC)c(OC)c(OC)c3)c(O)c(C(=O)C(C)CC)c2O)c(O)c(C)c1OC]

### 1-(3-(3-butyryl-2,6-dihydroxy-4-methoxy-5-methylbenzyl)-2,4,6-trihydroxy-5-(3,4,5 trimethoxybenzyl)phenyl)-2-methylbutan-1-one (30)

Method A: 37 mg, yield 67%. ¹H-NMR (600 MHz, CDCl₃): δ 6.45 (s, 2H), 3.86 (s, 2H), 3.82 (s, 3H), 3.79 (s, 6H), 3.72 (s, 3H), 3.08 (t, J=7.2 Hz, 2H), 2.12 (s, 2H), 1.80 (m, 1H), 1.72 (m, 2H), 1.38 (m, 1H), 1.12 (d, J=6.7 Hz, 3H), 0.98 (d, J=7.3 Hz, 3H), 0.87 (d, J=7.3 Hz, 3H). ¹³C-NMR (150 MHz, CDCl₃): δ 211.1, 206.9, 161.7, 160.2, 159.5, 153.7, 136.9, 133.9, 112.1, 109.5, 107.8, 105.9, 104.9, 61.5, 60.8, 56.1, 45.8, 44.2, 29.0, 26.8, 18.1, 16.6, 16.4, 13.9, 11.9, 9.1. HR-MS (negative mode): calculated for [M−H]⁻ 625.2654; Found 625.2660.

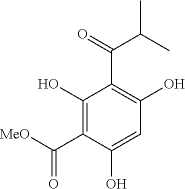 [SMILES: COC(=O)c1c(O)cc(O)c(C(=O)C(C)C)c1O]

### Methyl 2,4,6-trihydroxy-3-isobutyrylbenzoate (32)

4.23 g, yield 69%. ¹H NMR (600 MHz, CDCl₃) δ 14.46 (s, 1H), 6.01 (s, 1H), 4.08 (s, 3H), 3.89 (m, 1H), 1.17 (d, J=6.6 Hz, 6H). ¹³C NMR (150 MHz, CDCl₃) δ 211.0, 171.6, 169.6, 104.0, 97.3, 92.4, 53.0, 39.6, 19.0. HR-MS (negative mode): calculated for [M−H]⁺ 253.0717; Found 253.0711.

General Procedure for A-B Ring Coupling by BF₃-Et₂O (Method C):

A solution of acryl compound (1 eq.) and benzyl alcohol (1 eq.) in dioxane (˜0.1M) under an argon atmosphere protection was stirred at room temperature. BF₃-Et₂O (1.1 eq.) was added and the reaction temperature was increased to 100° C. and kept for 1 h. After cooling, the reaction mixture was diluted with ethyl acetate and further washed with sat. NaHCO₃ solution. The mixture was washed three times with EtOAc. The combined EtOAc extracts were washed with brine and dried over anhydrous Na₂SO₄, evaporated in vacuo and the remaining residue was purified by silica gel column chromatography using n-hexane-EtOAc as eluent to obtain the compound.

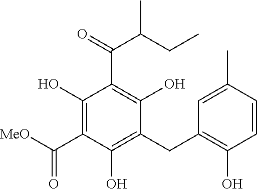 [SMILES: CCC(C)C(=O)c1c(O)c(Cc2cc(C)ccc2O)c(O)c(C(=O)OC)c1O]

### Methyl 2,4,6-trihydroxy-3-(2-hydroxy-5-methylbenzyl)-5-(2-methylbutanoyl)benzoate (33)

Method C: 78 mg, yield 78%. ¹H-NMR (600 MHz, CDCl₃): δ 7.26 (d, J=1.8 Hz, 1H), 6.89 (dd, J=6, 1.8 Hz, 1H), 6.75 (d, J=6 Hz, 1H), 4.10 (s, 3H), 3.82 (s, 2H), 3.75 (m, 1H), 3.60 (brs, 2H), 3.24 (s, 3H), 1.82 (m, 1H), 1.40 (m, 1H), 1.15 (d, J=6.6 Hz, 2H), 0.91 (t, J=7.8 Hz, 3H). ¹³C-NMR (150 MHz, CDCl₃): δ 211.5, 170.0, 168.4, 152.1, 132.2, 131.1, 129.0, 128.4, 125.4, 116.3, 107.9, 104.0, 92.5, 53.2, 46.3, 26.7, 22.6, 20.5, 16.4, 11.9. HR-MS (negative mode): calculated for [M−H]⁻ 387.1449; Found 387.1440.

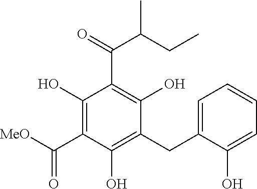 [SMILES: CCC(C)C(=O)c1c(O)c(Cc2ccccc2O)c(O)c(C(=O)OC)c1O]

### Methyl 2,4,6-trihydroxy-3-(2-hydroxybenzyl)-5-(2-methylbutanoyl)benzoate (34)

Method C: 24 mg, yield 32%. ¹H NMR (600 MHz, CDCl₃) δ 7.48 (dd, J=7.6, 1.7 Hz, 1H), 7.44 (s, 1H), 7.14-7.05 (m, 1H), 6.86 (dd, J=8.1, 1.2 Hz, 1H), 6.82 (td, J=7.4, 1.3 Hz, 1H), 4.10 (s, 3H), 3.87 (s, 2H), 3.76 (h, J=6.6 Hz, 1H), 1.83 (dqd, J=13.3, 7.5, 5.9 Hz, 1H), 1.48-1.35 (m, 1H), 1.16 (d, J=6.7 Hz, 3H), 0.92 (t, J=7.4 Hz, 3H). ¹³C NMR (151 MHz, CDCl₃) δ 211.7, 170.2, 168.5, 154.7, 132.1, 128.1, 125.8, 120.1, 116.6, 107.9, 104.2, 92.8, 53.5, 46.5, 26.8, 22.9, 16.6, 12.1. HR-MS (positive mode): calculated for [M+H]⁺ 375.1439; Found 375.1450.

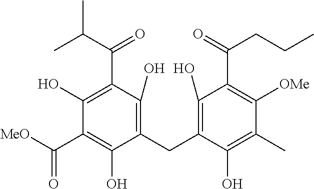 [SMILES: CCCC(=O)c1c(O)c(Cc2c(O)c(C(=O)OC)c(O)c(C(=O)C(C)C)c2O)c(O)c(C)c1OC]

### methyl 3-(3-butyryl-2,6-dihydroxy-4-methoxy-5-methylbenzyl)-2,4,6-trihydroxy-5-isobutyrylbenzoate (35)

Method C: 129 mg, yield 75%. ¹H-NMR (600 MHz, CDCl₃): δ 3.99 (s, 3H), 3.98 (m, 1H), 3.82 (s, 2H), 3.70 (s, 3H), 3.60 (brs, 1H), 3.06 (t, J=7.2 Hz, 2H), 2.09 (s, 3H), 1.71 (m, 3H), 1.17 (d, J=7.2 Hz, 6H), 0.96 (t, J=7.8 Hz, 3H). ¹³C-NMR (150 MHz, CDCl₃): δ 210.9, 205.8, 171.0, 166.2, 163.0, 160.4, 159.1, 158.9, 110.7, 107.9, 106.9, 104.6, 102.6, 93.7, 60.5, 51.8, 43.3, 38.4, 18.1, 17.2, 14.8, 12.9, 8.1. HR-MS (negative mode): calculated for [M−H]⁻ 489.1766; Found 489.1761.

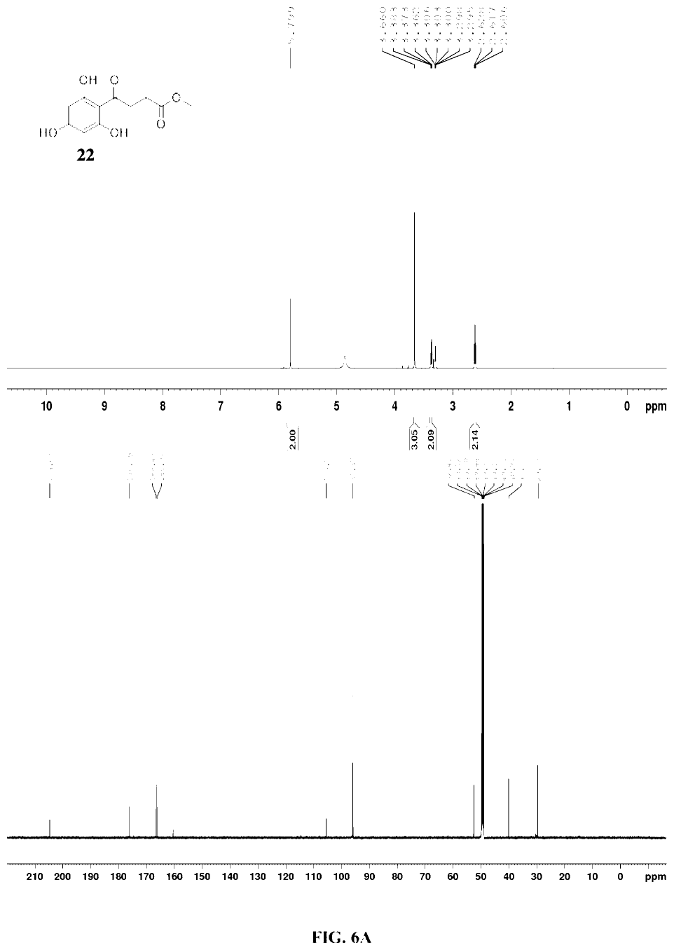

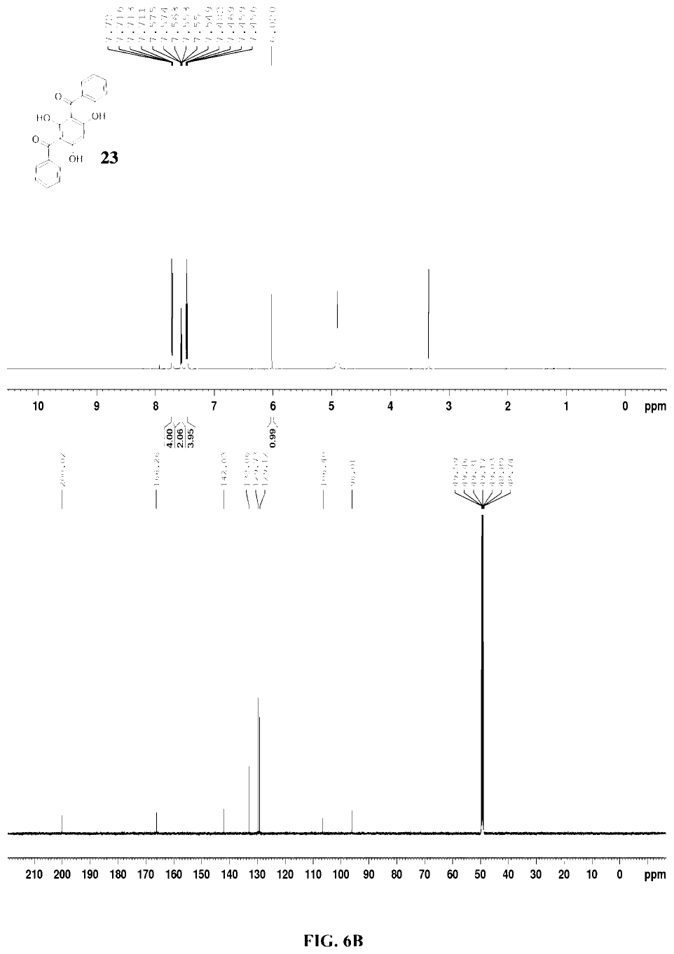

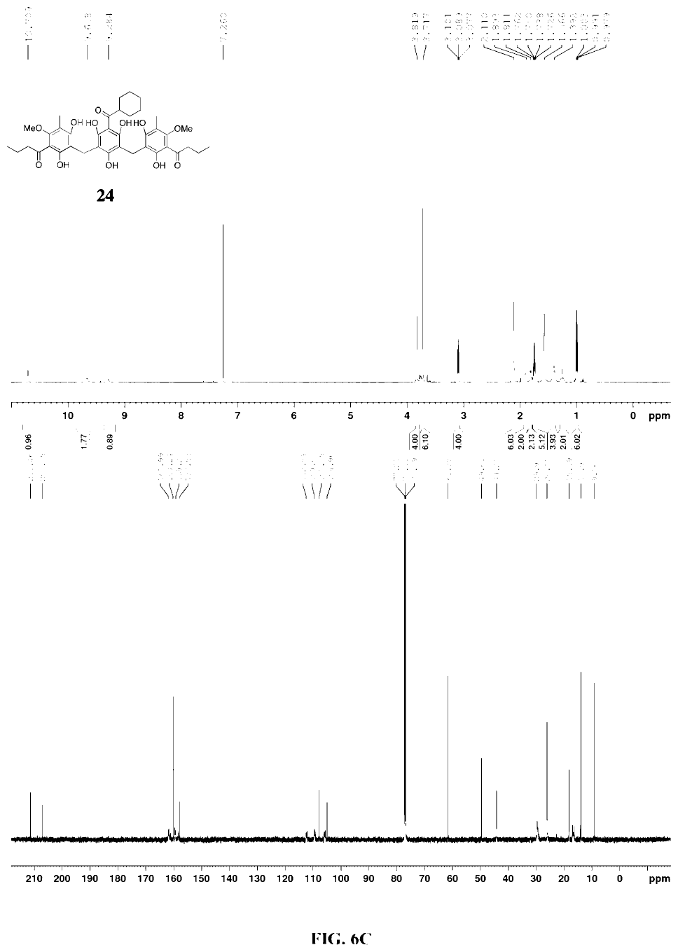

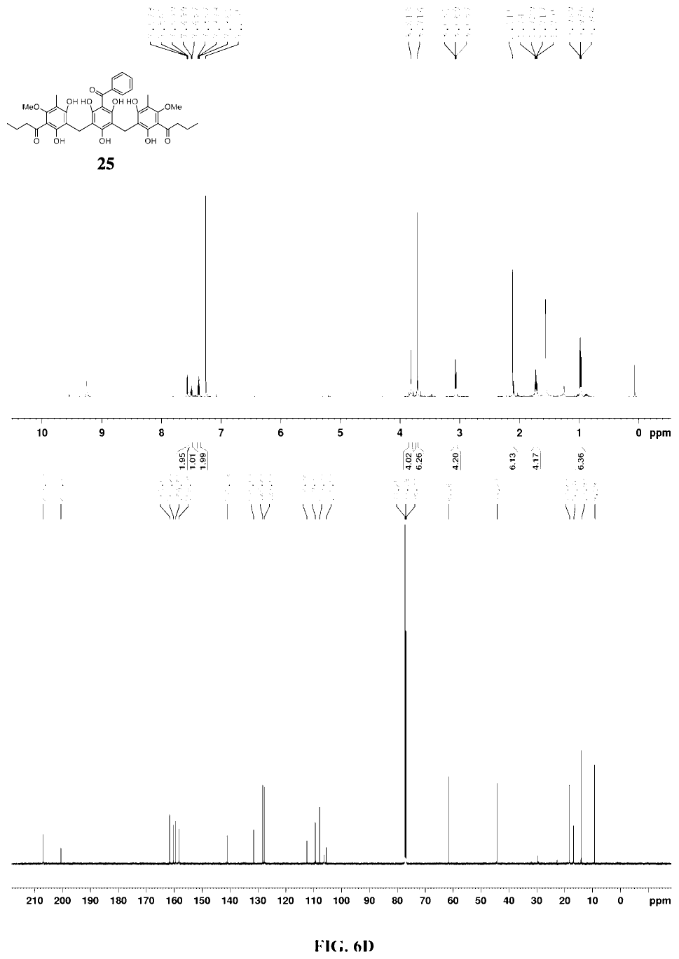

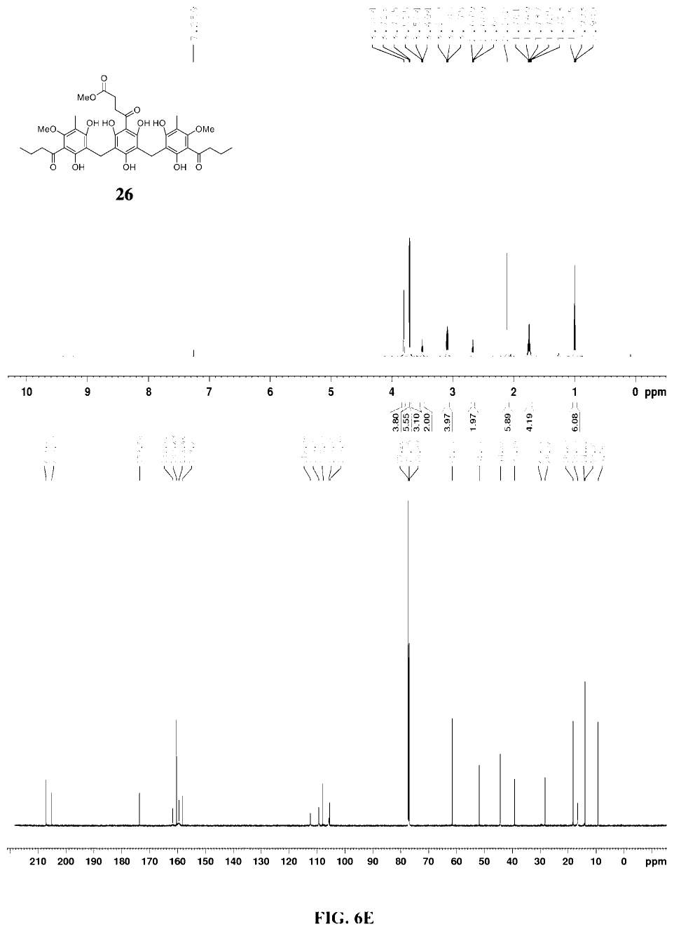

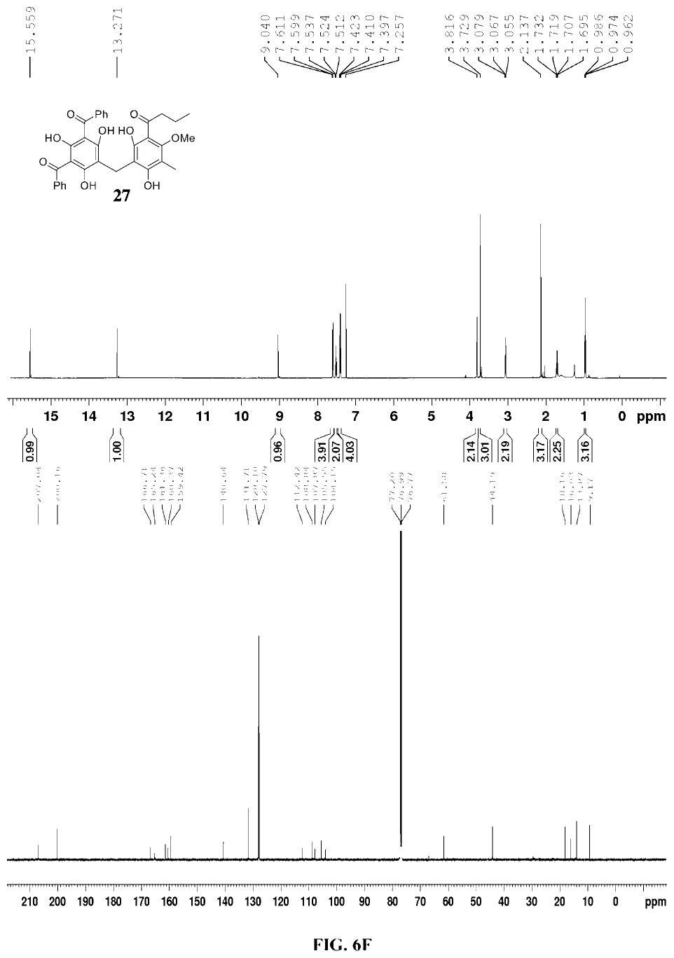

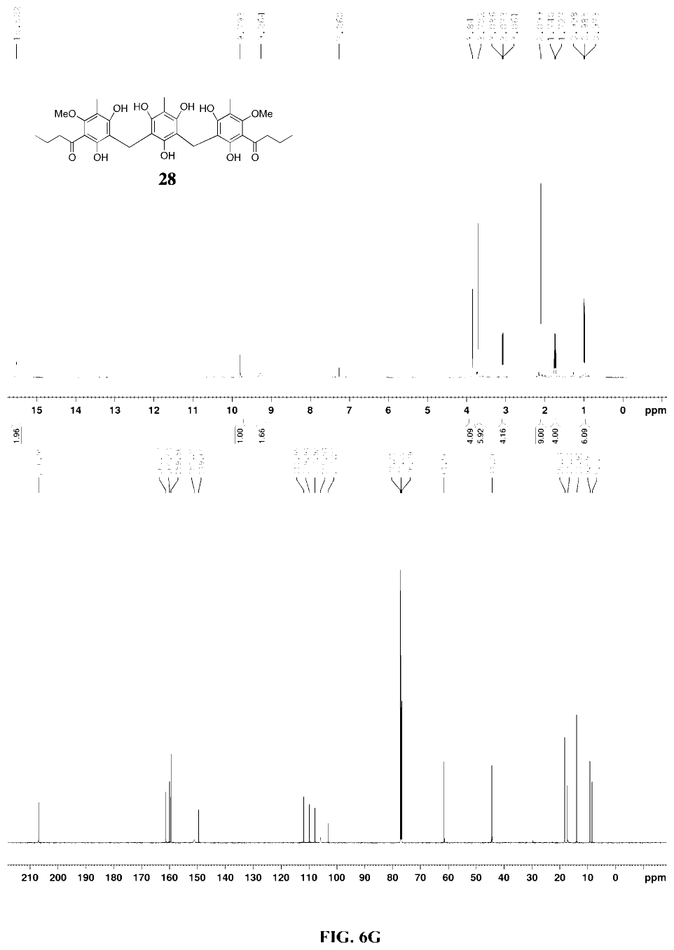

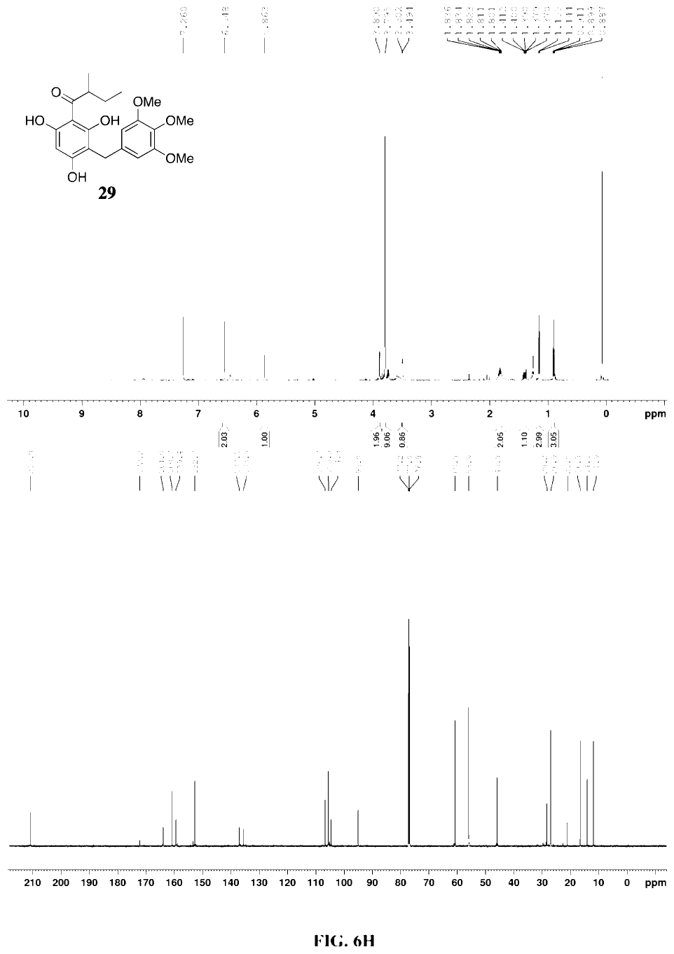

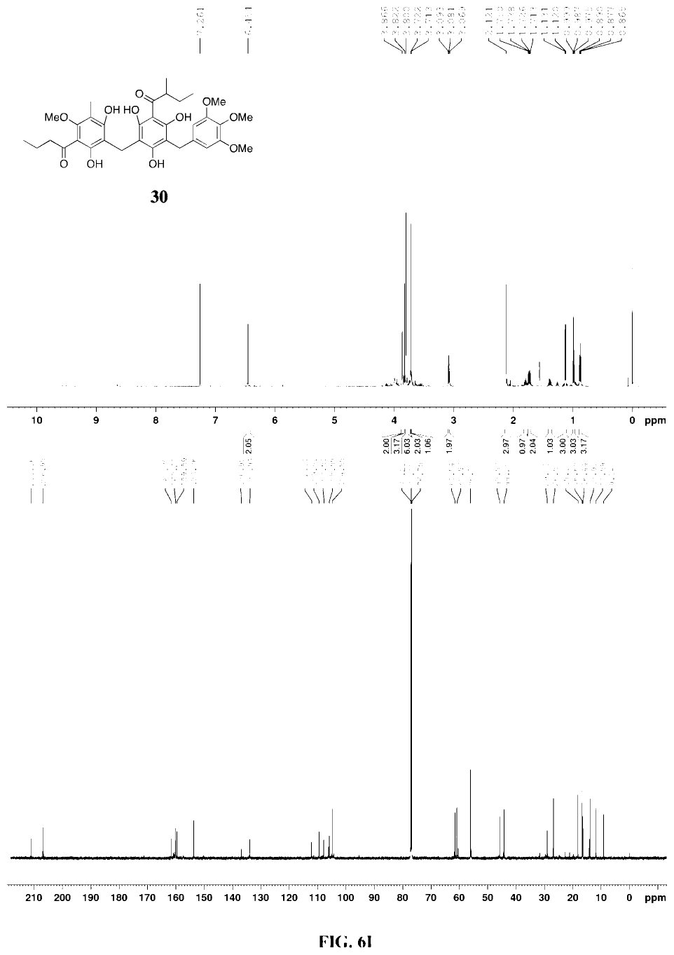

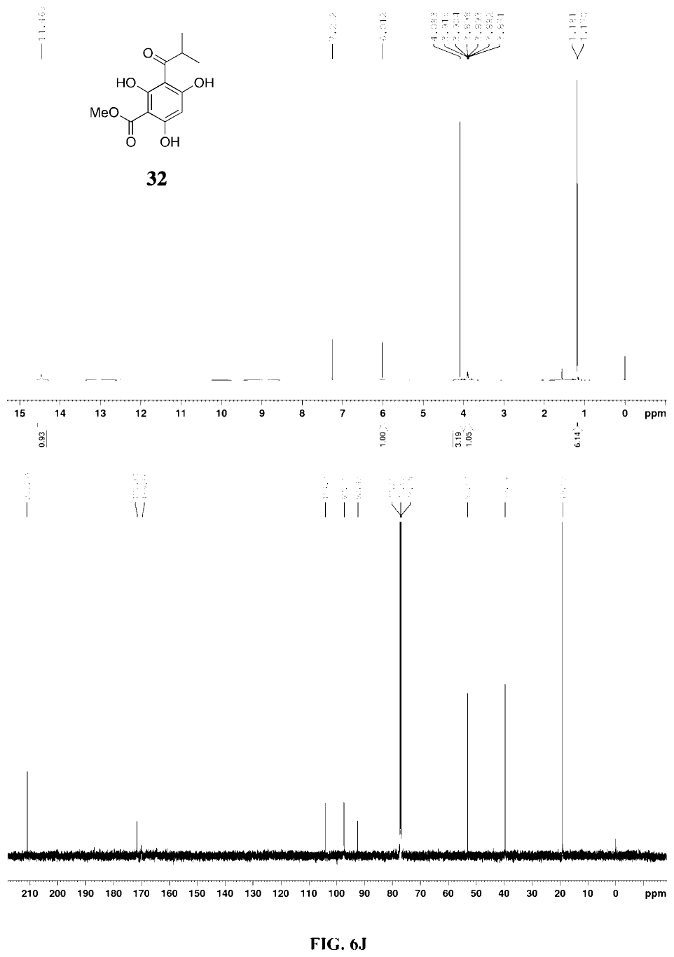

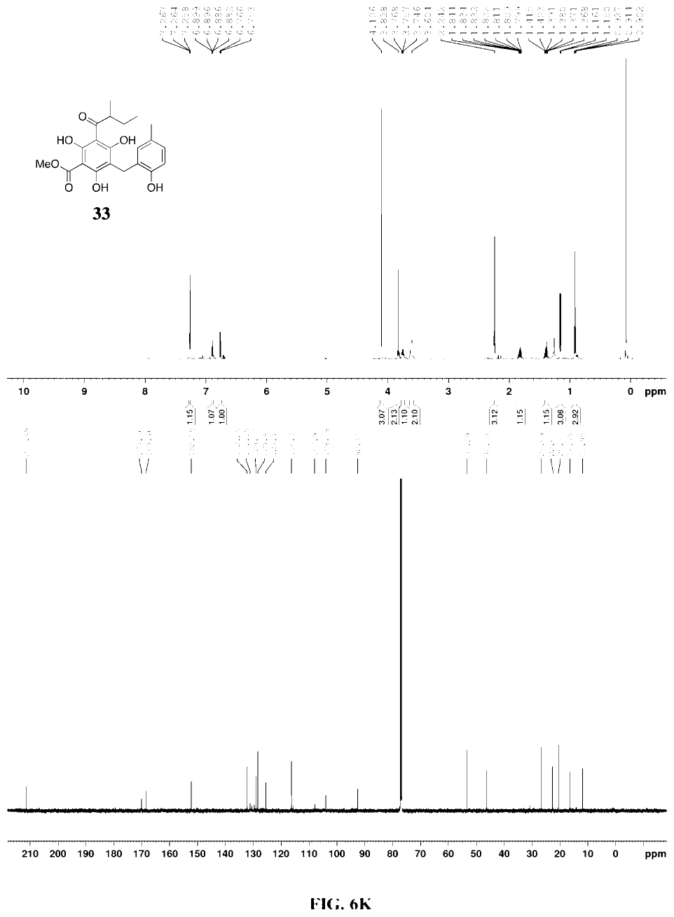

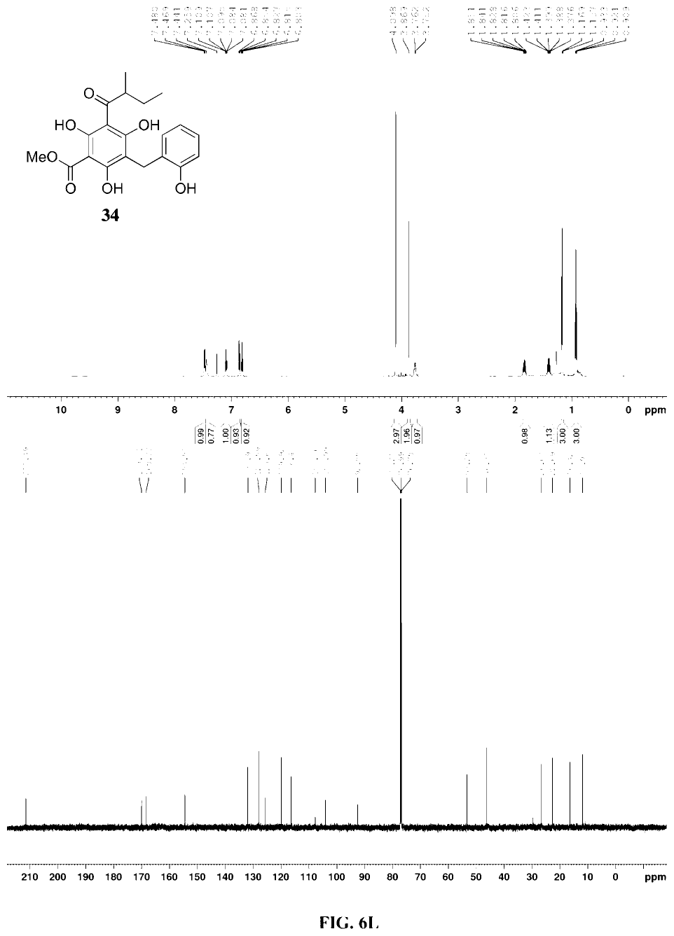

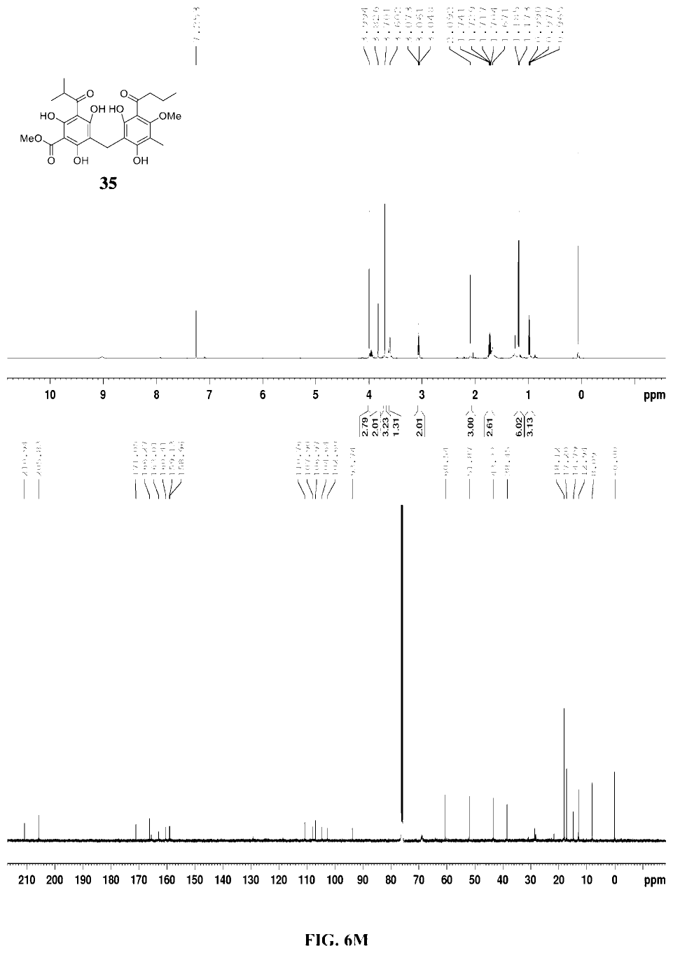

The spectrum of some above AGB derivatives are shown in FIGS. 6A-6M.

### Cell Culture

SUNE1 (nasopharyngeal carcinoma), AGS (gastric adenocarcinoma), HeLa (cervix adenocarcinoma), HepG2 (hepatocellular carcinoma), U-87 MG (human glioblastoma cell), U2OS (osteosarcoma), MCF-7 (breast adenocarcinoma), MDA-MB-231 (breast adenocarcinoma), HCT116 (colorectal carcinoma), SW480 (colorectal adenocarcinoma), NCI-H460 (large cell lung carcinoma), NCI-H1650 (adenocarcinoma lung carcinoma), HCC827 (adenocarcinoma lung carcinoma), A549 (lung carcinoma), and CCD-19Lu (human lung fibroblast normal cell) were all obtained from American Type Culture Collection (ATCC, Manassas, VA, USA). Minimum Essential Media (MEM, Gibco®, ThermoFisher Scientific), Dulbecco's Modified Eagle Medium (DMEM, Gibco®, ThermoFisher Scientific), and RPMI-1640 Medium (Gibco®, ThermoFisher Scientific) supplemented with 10% Fetal Bovine Serum (Gibco®, ThermoFisher Scientific), and 100 U/mL penicillin-streptomycin (Gibco™, ThermoFisher Scientific) were used for cell culture. Cells were placed into an incubator with 5% CO₂ that temperature was set at 37° C. For subculturing cells, 0.05% Trypsin-EDTA (Gibco®) was used for detaching and a subcultivation ratio of 1:5 was used.

### Cell Viability Assay

The cell viability assay was performed by a typical MTT assay. Briefly, 5*10³ cells were seeded in the 96-well flat bottom plate and allowed to attach overnight. After drug treatment with a serial dilution from 100 μM for 48 h, 10 μL of MTT solution (5 mg/ml prepared in PBS, filtered) was added into each well and incubate for 4 h at 37° C. The formed formazan was then dissolved with SDS solution (10% SDS in 10 mM HCl) and followed the determination of absorbance at 590 nm by a microplate reader. The change in cell viability was then calculated for each drug concentration, then normalized to control. Dose response curves from each assay were fit to a Boltzmann sigmoidal function (GraphPad Prism8, version8.0.0). The cytotoxicity of AGB and its derivatives was determined by the calculation of IC₅₀ value (half maximal inhibitory concentration) from fitted curves, which was the concentration of drug induced 50% cell viability.

### Cell Colony Formation Assay and Wound Healing Assay

For the colony formation assay, cells were treated with indicated concentrations of AGB for 8 h, then AGB was then removed and 200 cells were trypsinized and seeded into a new 12-well cell culture plate containing 1.5 ml of complete cell growth medium. After cells were grown for 14 days, cells were then fixed with ethanol followed a staining by crystal violet (0.05%, w/v, in water) for 15 min. The cell colonies were captured under the microscope and the number of colonies was calculated by ImageJ (version 1.50i, NIH, USA, internet site imagej.nih.gov).

For wound healing assay in the present study, the cells were grown to approximately 80% confluence in 12-well plates in the complete growth medium and then a straight line was scraped at the central part of the each well by a 200 μL pipette tip. After washing the detached cells by PBS, the cells continued to culture in the complete growth medium for 24 h with different concentration of AGB or DMSO as vehicle control. The gaps made by pipette tip were measured at 0 h and 24 h, respectively. Each condition was performed in triplicate.

### Sphere Formation Assay

NCI-H460 cells were seeded at 3,000 cells/ml per well in specific sphere formation medium (DMEM/F12 supplemented with 20 ng/ml EGF, 20 ng/ml bFGF and 1×B-27) with or without AGB (DMSO as vehicle, 0.5 and 1.0 μM of AGB for the assay) in a 6-well Ultra Low Cluster Plate (Costar®). The concentration of DMSO was normalized to 0.5% in all wells and was 0.5%. The cells were incubated at 37° C. for five days and the spheres were photographed under an inverted microscope at 40× magnification. Primary tummorsphere were collected and enzymatically dissociated using trypsin to generate the secondary tummorsphere. The dissociated cells were re-plated at a density of 2,000 cells/mL for subsequent passages. The images were captured and four fields were randomly chosen and the relative areas occupied by the spheres were measured using ImageJ (National Institutes of Health, Bethesda, MD). Representative images were obtained from three independent experiments.

For IC₅₀ determination of sphere, similar procedures were performed as mentioned above. Briefly, cells were plated in 96-well clear flat bottom ultra-low attachment cell culture plates (Corning®) with a density of 5*10³ cells into each well with specific sphere formation medium (DMEM/F12 medium supplemented with 20 ng/mL EGF, 20 ng/mL bFGF, 2% B27). The cells were then treated with AGB at different concentrations, ranging from 0.3 to 100 μM. After 3 days of treatment, the formed tumorspheres in each well were monitored under microscope. The MTT was then applied to determine the cell viability and the cell viability of AGB-treatment group was calculated via normalization to control groups and the IC₅₀ value was calculated from the plot curve that fitted to a Boltzmann sigmoidal function.

### Animal Studies

In vivo anticancer efficacy of AGB was evaluated using the human tumor xenograft model, in which human cancer cells were inoculated into immune-deficiency mice. Briefly, twelve six-week-old (16-18 g) BALB/c nude mice were randomly divided into two groups and both received subcutaneous injection of NCI-H460 cells (2 million cells in 0.1 mL PBS) at its right flank of mice and tumor became palpable after three days of injection. Mice were received intravenous (i.v.) injections of AGB or vehicle control (10% DMSO and 0.05% Tween 80 in normal saline) every two days in two weeks. The length and width of tumor were measured by Vernier caliper and the body weight was monitored every two or three days. At the end of experiment, mice were sacrificed and decapitated. The liver, kidney, and tumor were collected and fixed in 4% formaldehyde for further haematoxylin and eosin (H&E) staining. All animal experiments in this project received human care and study protocols complied with the guidelines of the Laboratory Animal Services Centre of the University of Hong Kong. Tumor sizes and body weight were recorded before the drug injection every time and tumor volume (V) was calculated by the formula V=LW²/2, where L is tumor length and W is tumor width. The relative tumor volume that normalized to day 0 was used to monitor the tumor growth. Data in each group was shown as mean±standard deviation.

### Maximum Tolerated Dose (MTD) Determination on Mice

MTD is the highest dose of a drug which exerts its desired effectiveness but simultaneously does not induce observable side adverse effects [14]. The MTD test in this study was done in collaboration with Tianjin Institute of Pharmaceutical Research (TIPR). The test was performed to determine the MTD of AGB on ICR mice following single intravenous (i.v.) injection of 12.8 mg/kg and 16.0 mg/kg and to evaluate its toxicity within 14-day recovery. A total of 80 mice were randomly assigned into four groups (10 male and 10 female mice in each group) in this study. Four groups included 16.0 mg/kg of AGB group, 12.8 mg/kg of AGB group, vehicle control group (10% DMSO+0.8% Tween-80 in normal saline), and the blank control group (normal saline).

After a single intravenous administration, the general toxicity of all animals in each group was observed in each group. Then, the body weights of surviving animals were measured on the 1^{st}, 3^{rd}, 7^{th} and 14^{th} days, respectively. At the end of experiment, the hematology, blood biochemistry, anatomical examination, and histopathology examination were performed in both vehicle control group and the MTD group (12.8 mg/kg). The data were presented as the mean±standard deviation.

### Data Statistical Analysis

Data were expressed as mean±standard deviation (SD). Statistical analysis between multiple groups was performed using ANOVA, followed by Tukey-Kramer tests. And differences between two groups were determined by Student's t-tests. Statistical significance was accepted with a two-tailed p-value under 0.05 (α=0.05).

### Results

### Total Synthesis of AGB

It is important and meaningful to overcome the limitation of amount scarcity and provide a feasible route for the study of SAR of AGB. Total synthesis reported for AGB to date had low yield and involved complex reactions (0.6% yield, Li et al. Acta Chimica Sinica, 1978, 36, 43-48; 0.3% yield, Zhu et al. Acta Pharmaceutica Sinica, 1989, 24, 431-437). To improve the total synthesis of AGB and its derivatives, two strategies were developed in the present study.

The first synthetic route (Scheme 1) starts from 2, 4, 6-trihydroxyl benzoic acid (5) to produce the hydroxyl-methylation compound (6). Methyl group was then introduced on the benzene ring to produce compound (8) via Vilsmeier-Haack reaction. Following acylation of compound (8), decarboxylation was performed to produce intermediate (4). Calculating from 2, 4, 6-trihydroxyl benzoic acid (5), the overall yield of AGB using synthetic route 1 was about 7.3% (Scheme 1). In this synthetic route, the sequence of steps for acylation and methylation was changed to obtain the key intermediates, and Eschenmoser methenylation reaction was employed in the last step to couple three subunits.

The second synthetic route (Scheme 2) starts from phloroglucinol (11) to produce 2,4,6-trihydroxyl methylbenzene (13) via Vilsmeier-Haack reaction, followed by the acylation of compound (13) via Friedel-Crafts reaction. Prior to methylation of one of the hydroxyl groups of compunds (13), methyl chloromethyl ether (MOMCl) was used to protect two of its hydroxyl groups. Introducing protection groups on two of the hydroxyl groups of compound (13) allows highly selective methylation because of the steric hindrance. Methylation of protected compound (13) was performed using dimethyl sulfate, followed by deprotection of MOM groups by heating in 1M HCl methanol solution. Calculating from phloroglucinol (11), the overall yield of AGB using synthetic route 2 was about 20.2% (Scheme 2).

All the steps in the first and second synthetic routes are easy to perform and have with a significantly higher overall yield compared to previously reported synthesis (0.6%, Li et al. Acta Chimica Sinica, 1978, 36, 43-48; 0.3%, Zhu et al. Acta Pharmaceutica Sinica, 1989, 24, 431-437). The structure of AGB was characterized by 1H, 13C-NMR and HRMS along with 2D-NMR.

Several derivatives of AGB were synthesized to further reveal the structure and activity relationship (SAR). The different lengths of the acyl group on middle subunit were designed and synthesized using the according acyl chlorides. In this work, the intermediates 17-19 were obtained with the carbons of 1, 3, and 7, respectively. After coupling with intermediate 16, the three AGB's derivatives were generated and named agrimol H, agrimol C, and agrimol L, respectively. The detailed synthetic routes are shown in Scheme 3.

### In Vitro and In Vivo Anticancer Activities

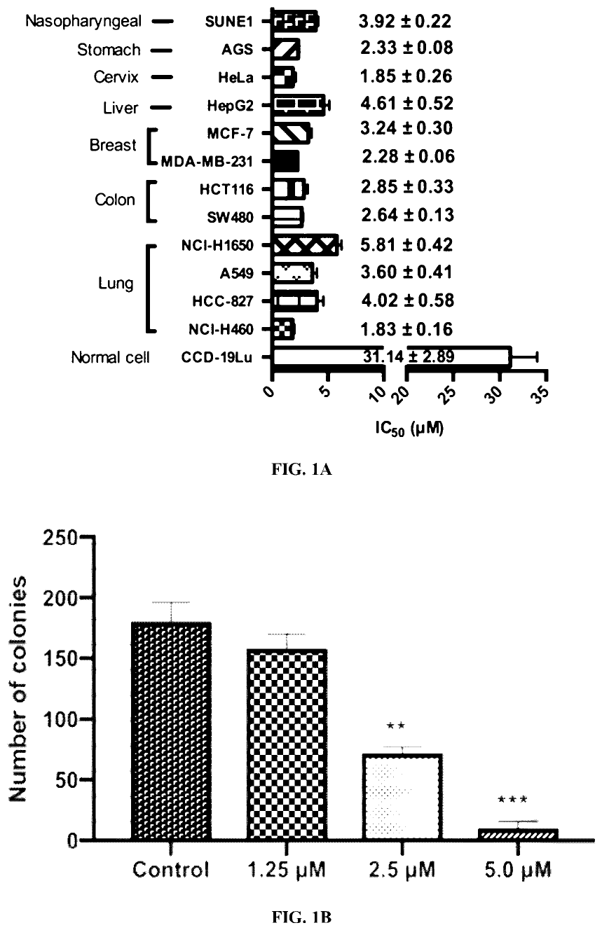

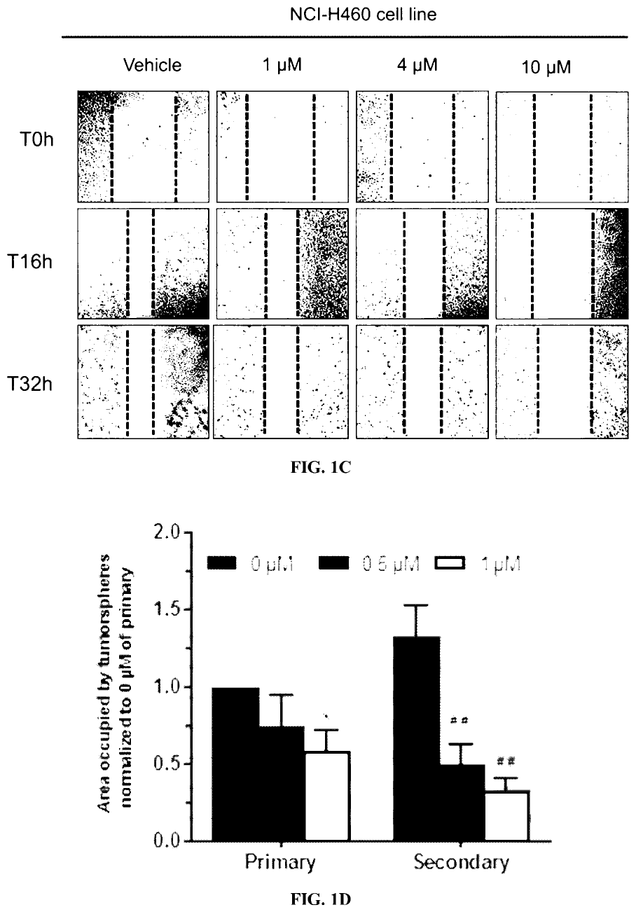

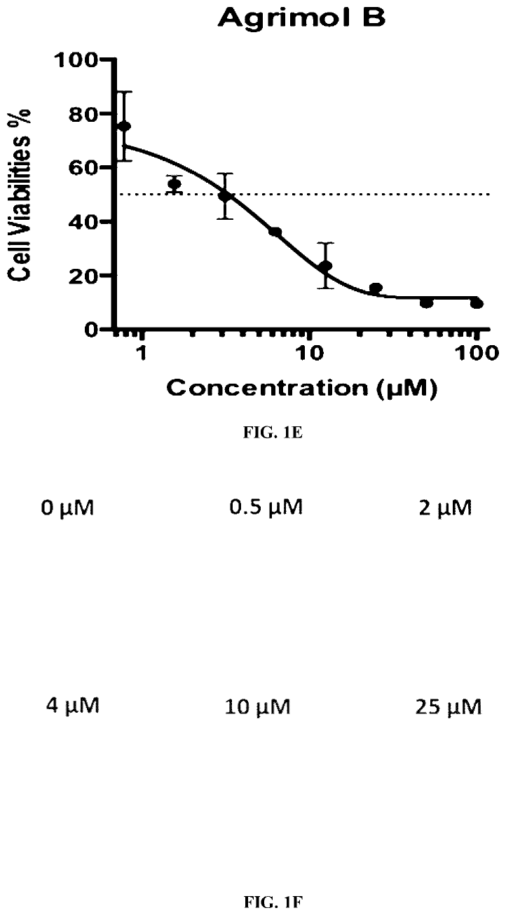

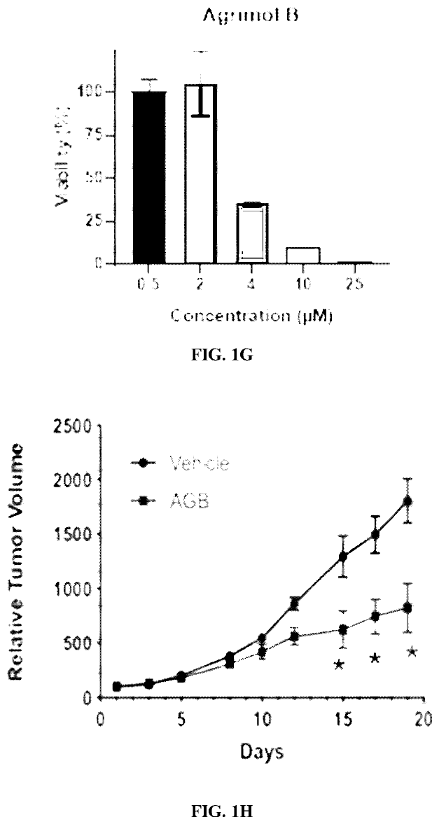

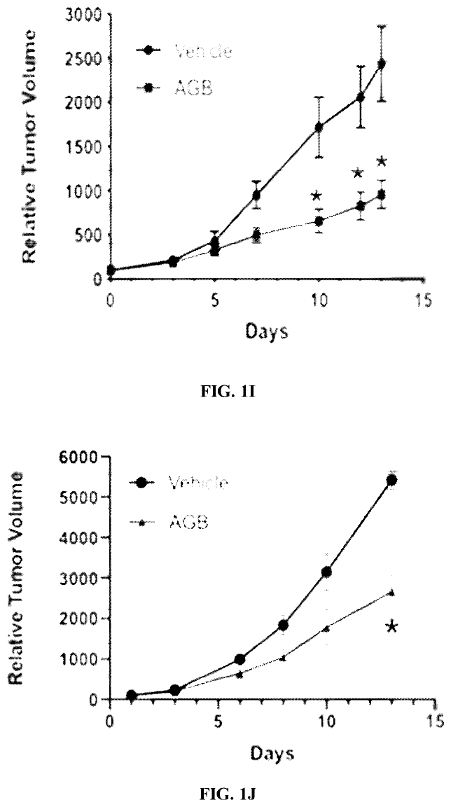

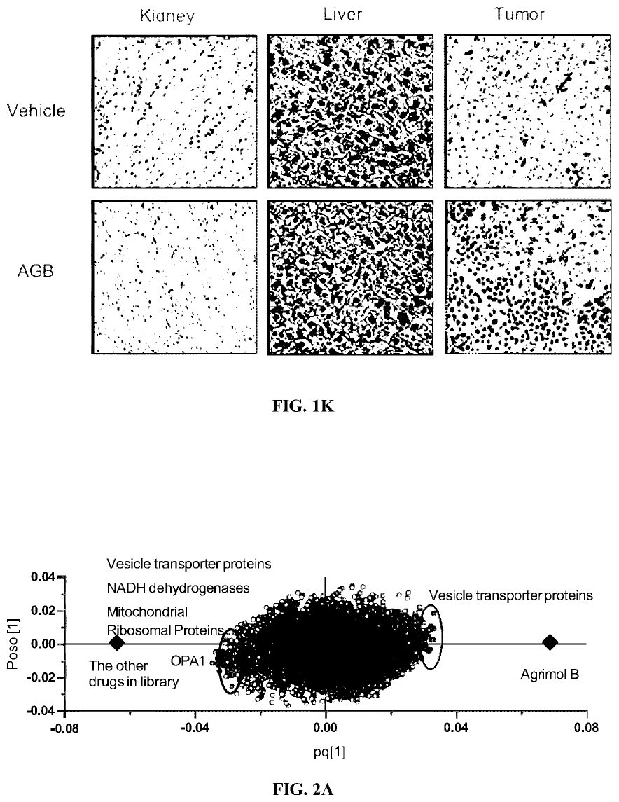

The cytotoxicity of AGB against a normal cell (CCD-19Lu) and several human cancer cell lines, including breast adenocarcinoma (MDA-MB-231 & MCF-7), hepatocellular carcinoma (HepG2), cervical cancer (HeLa), stomach adenocarcinoma (AGS), colon cancer (HCT116 & SW480), nasopharyngeal carcinoma (SUNE-1), and lung carcinoma (H460, HCC827, H1650, and A549) cell line was determined using MTT assay. Compared to the normal human lung fibroblast CCD-19Lu cells, results from the MTT assay showed that AGB exerted a significant selective anti-proliferative activity against cancer cells, with IC50 values below than 10 μM (FIG. 1A). For example, after a 48-h treatment, AGB exhibited potent cytotoxicity with IC₅₀ values at about micro-molar concentrations for the tested cancer cell lines, ranging from 1.83 μM to 5.81 μM (FIG. 1A). The IC₅₀ value for CCD-19Lu was 31.14 μM for 48-h treatment, which is much higher than the average IC₅₀ values on cancer cells.

The proliferative inhibitory effect of AGB on NCI-H460 cells was further examined by the clonogenic assay. NCI-H460 cells were treated with different concentration of AGB for 8 h followed trypsinization and re-seeding into a fresh normal growth RPMI-1640 medium. Cell colonies were stained and counted after two weeks of treatment (images not shown). As shown in FIG. 1B, the colonies numbers were found to be significantly reduced when the AGB concentration was over 2.5 μM and the effects were displayed in a dose-dependent manner. To investigate the effect of AGB on cell migration ability, the scratched cell wound healing assay was performed. NCI-H460 cells were seeded into a 24-well cell culture plate at a density about 80% confluent and a scratch was made on cell monolayer. The cells were then monitored in the presence or absence of AGB (1 μM, 4 μM, and 10 μM) and images were captured at 16 h and 32 h. As shown in FIG. 1C, the cells in vehicle and 1 μM of AGB groups proliferated faster and the width of the scratch wound significantly decreased. For the cells treated with 4 μM or 10 μM of AGB, wound closure was inhibited and a larger size of wound gap was observed (FIG. 1C). Using clonogenicity assay and the wound healing assay, AGB showed a significant reduction of cell colony formation as well as cell migration at around 4 μM (FIG. 1B and FIG. 1C).

Cancer stem cells (CSCs) are a self-renewing, highly tumorigenic subpopulation of tumor cells. They play a critical role in the initiation and progression of cancer. To assess the effect of AGB on tumorsphere self-renewal ability, NCI-H460 spheres were grown in ultra-low attachment plates with cancer stem cell media (DMEM-F12, supplemented with B27, EGF, and FGF) and treated with AGB for two successive passages. The treatment with AGB significantly decreased the number of tumorsphere in each passage of tumorsphere culture. The tumorsphere forming efficiency of primary tumorspheres was reduced upon treatment with 1.0 μM AGB (40.6% inhibition, p<0.05). Similarly, the tumorsphere forming efficiency in secondary tumorspheres was decreased with 0.5 μM or 1.0 μM AGB (54.7% inhibition, p<0.01 and 69.7% inhibition, p<0.01) (FIG. 1D). Moreover, when using 96-well round-bottom cell culture plate for observation, the size of formed tumorspheres was found to have an observable reduction upon the treatment of AGB for 72 hours, compared to control group and group treated with 0.5% DMSO (images not shown). Using MTT assay, the IC₅₀ value of AGB for cancer stem cells was determined to be 4 μM after a 72-h treatment (FIG. 1E).

To better simulate the physiological structure and function of in situ tissue, the effect of AGB on the growth of patient-derived organoids (PDOs) isolated from a lung cancer patient's metastatic tumor was evaluated. Significant inhibition of viability of PDO with obvious apoptotic characteristics was observed upon treatment with AGB at 4-25 M in a concentration dependent manner (FIG. 1F). AGB showed a maximal viability loss over 99% (FIG. 1G).

The in vivo antitumor potential of AGB using the xenograft mouse model was assessed. Two different cancer cells (NCI-H460 and A549) were inoculated subcutaneously into the nude mice. Non-obese diabetic severe combined immunodeficient (NOD/SCID) mice were used for stem-like NCI-H460 cells. The mice were then treated by tail intravenous injection with vehicle or AGB (3 mg/kg). AGB significantly inhibited the tumor growth on all three types of xenograft models (A549, NCI-H460, and stem-like NCI-H460), with the inhibition rates as 60%, 57% and 64%, respectively (FIGS. 1H-1J). Moreover, the 3 mg/kg of AGB dosage did not show any observable toxicity as no significant change in body weight of mice was observed (data not shown). To further evaluate the safety of AGB, hematoxylin-eosin (H&E) staining of the kidney, liver, and tumor samples were performed (FIG. 1K). The necrotic area (circled by dashed line) in the tumor tissue from mice treated with AGB was much larger than that in vehicle group. The liver and kidney, as the two most important organs for drug metabolism, did not show any obvious change after treatment with AGB.

The maximum tolerance test of AGB was performed, using two dosages: 12.8 mg/kg and 16.0 mg/kg. No symptoms of toxic reactions were observed on the day of dosing and during the post-drug recovery period except 16.0 mg/kg treatment group. For the 16.0 mg/kg treatment group, most animals (13/20) reduced activities in about 30 minutes after AGB administration, and some mice appeared prone (3/20) and ptosis (1/20) in about 1 hour after AGB administration. All of the symptoms did not reverse completely on the first day, and one mouse died on the second day. The rest of mice gradually recovered after one week and did not show any obvious symptoms of toxicity. The body weights of mice in blank control group, vehicle control group, 12.8 mg/kg treatment group, and 16.0 mg/kg treatment group were monitored (Tables 1 and 2). In the vehicle control group, the average body weight of all mice on the day of administration and during the recovery period after drug administration showed no obvious abnormalities compared with the blank control group. In the 12.8 mg/kg treatment group, the average body weight of male mice at 3 and 7 days after AGB administration was lower than those of the blank control but showed a slow increase; the average body weight of female mice at 1 and 3 days after AGB administration was lower than those of the blank control but recovered at days 7 and 14. No obvious abnormality of body weight was observed at each time checkpoint. In the 16.0 mg/kg treatment group, the average body weights of male mice and female mice 1, 3, and 7 days after AGB administration were lower than that of the blank control group, but gradually recovered after 14 days following AGB administration.

TABLE 1
The body weight of male mice (mean ± SD, g)

| | Blank | Vehicle | | |
|---|---|---|---|---|
| | Control | Control | 16.0 mg/kg | 12.8 mg/kg |
| Days | (n = 10) | (n = 10) | (n = 10) | (n = 10) |
| d 0 | 21.38 ± 1.04 | 21.53 ± 0.67 | 21.24 ± 0.58 | 21.16 ± 1.07 |
| d 1 | 22.53 ± 1.14 | 22.44 ± 0.94 | 19.64 ± 1.15 ** | 21.27 ± 1.46 |
| d 3 | 24.89 ± 1.73 | 25.24 ± 1.43 | 20.04 ± 1.42 ** | 21.89 ± 1.51 ** |
| d 7 | 28.36 ± 2.58 | 28.82 ± 1.72 | 24.25 ± 1.48 ** | 25.64 ± 1.54 ** |
| d 14 | 33.56 ± 3.36 | 33.34 ± 1.81 | 31.48 ± 1.73 | 31.60 ± 2.37 |

TABLE 2
The body weight of female mice (mean ± SD, g)

| | Blank | Vehicle | | |
|---|---|---|---|---|
| | Control | Control | 16.0 mg/kg | 12.8 mg/kg |
| Days | (n = 10) | (n = 10) | (n = 10) | (n = 10) |
| d 0 | 20.45 ± 0.84 | 20.38 ± 0.54 | 20.57 ± 0.90 | 20.52 ± 1.00 |
| d 1 | 21.21 ± 1.02 | 20.71 ± 0.77 | 18.76 ± 0.89 ** | 20.60 ± 1.18 |
| d 3 | 22.26 ± 1.17 | 22.49 ± 0.95 | 19.61 ± 1.50 ** | 21.14 ± 0.99 |
| d 7 | 23.76 ± 1.69 | 23.95 ± 1.24 | 22.76 ± 1.26 | 23.68 ± 1.83 |
| d 14 | 26.48 ± 2.48 | 26.47 ± 1.61 | 25.61 ± 0.87 | 26.38 ± 1.63 |

Hematological examination (Table 3) and serum biological examination (Table 4) was also performed for the vehicle control group and 12.8 mg/kg treatment group. As shown in Table 3, on day 14 after AGB administration, the E, E %, RBC, HGB, and HCT decreased while the N %, PLT, Ret, and Ret % increased in the 12.8 mg/kg treatment group compared with the vehicle control group. Although the differences were not significant, the changes in the above markers may indicate possible impact of high-dose AGB on the blood system. As shown in Table 4, there were no obvious abnormalities in the 12.8 mg/kg treatment group compared with the vehicle control group 14 days after AGB administration.

TABLE 3
Hematological examination of samples from
vehicle and 12.8 mg/kg treatment group.

| Indicators | Vehicle control n = 10 | 12.8 mg/kg n = 10 | |
|---|---|---|---|
| WBC(×10\hat{ }9/L) | 6.46 ± 1.53 | 5.44 ± 1.66 | |
| N□ × 10\hat{ }9/L□ | 0.73 ± 0.25 | 0.78 ± 0.28 | |
| L□ × 10\hat{ }9/L□ | 4.97 ± 1.20 | 4.22 ± 1.32 | |
| M□ × 10\hat{ }9/L□ | 0.11 ± 0.04 | 0.09 ± 0.03 | |
| E□ × 10\hat{ }9/L□ | 0.55 ± 0.29 | 0.29 ± 0.11 | * |
| N(%) | 11.27 ± 2.72 | 14.24 ± 2.15 | * |
| L(%) | 77.09 ± 3.80 | 77.53 ± 2.05 | |
| M(%) | 1.78 ± 0.59 | 1.64 ± 0.46 | |
| E(%) | 8.38 ± 3.71 | 5.48 ± 1.64 | * |
| RBC(×10\hat{ }12/L) | 9.76 ± 0.37 | 9.11 ± 0.45 | ** |
| HGB(g/L) | 149.60 ± 4.86 | 138.20 ± 9.38 | ** |
| HCT(%) | 51.15 ± 0.75 | 47.54 ± 2.21 | ** |
| MCV(fL) | 52.49 ± 1.96 | 52.25 ± 2.16 | |
| MCH(pg) | 15.37 ± 0.80 | 15.20 ± 0.97 | |
| MCHC(g/L) | 292.70 ± 10.55 | 291.00 ± 20.17 | |
| PLT(×10\hat{ }9/L) | 1408.70 ± 185.99 | 1733.80 ± 371.53 | * |
| Ret(×10\hat{ }12/L) | 0.66 ± 0.08 | 0.81 ± 0.09 | ** |
| Ret(%) | 6.73 ± 0.71 | 8.83 ± 0.74 | ** |

Notes:
White blood cells (WBC), neutrophils (N), lymphocytes (L), monocytes (M), eosinophils (E), red blood cells (RBC), hemoglobin (HGB), hematocrit (HCT), mean corpuscle volume (MCV), mean corpuscle hemoglobin (MCH), mean corpuscle hemoglobin concentration (MCHC) and platelet count (PLT), reticulocyte (Ret)

TABLE 4
Serum biochemical examination of samples from
vehicle and 12.8 mg/kg treatment group.

| Indicators | Vehicle control n = 10 | 12.8 mg/kg n = 10 |
|---|---|---|
| ALT(U/L) | 30.47 ± 10.32 | 26.09 ± 2.23 |
| AST(U/L) | 79.15 ± 14.65 | 85.74 ± 15.96 |
| ALP(U/L) | 130.70 ± 29.99 | 153.67 ± 37.63 |
| BUN(mmol/L) | 5.86 ± 1.48 | 4.58 ± 2.24 |
| CREA(umol/L) | 34.77 ± 3.38 | 34.11 ± 3.61 |
| TP(g/L) | 50.80 ± 1.89 | 45.93 ± 13.71 |
| ALB(g/L) | 19.33 ± 1.04 | 17.73 ± 5.07 |
| GLU(mmol/L) | 10.23 ± 2.25 | 9.10 ± 3.07 |
| T.CHO(mmol/L) | 3.73 ± 1.42 | 3.68 ± 1.60 |
| T.BIL(umol/L) | 1.80 ± 0.31 | 1.75 ± 0.45 |
| TG(mmol/L) | 1.75 ± 0.16 | 1.41 ± 0.59 |
| CK(U/L) | 1088.86 ± 352.35 | 953.09 ± 480.50 |
| GGT(U/L) | 0.20 ± 0.27 | 0.07 ± 0.08 |
| LDH(U/L) | 368.87 ± 116.42 | 403.54 ± 92.68 |

The results discussed above demonstrate that AGB possess potent anticancer activities both in vivo and in vitro. AGB showed preferential anti-proliferative activity against cancer cells with a micro-molar concentration, inhibition of colony formation, cell migration, and blocking of the self-renewal ability of cancer stem-like cell. At the in vivo level, AGB showed an inhibitory effect on xenograft tumours growth with a low toxicity.

The structure-activity relationships (SAR) of AGB were also investigated. Cytotoxicity of five derivatives of AGB was examined using different cancer cell lines. As shown in Table 5, four different cancer cell lines were used for the evaluation of cytotoxicity (48-h treatment). Compounds AGF, AGS, and agrimol L (AGL) showed significantly lower cytotoxicity than AGB, while compounds agrimol H (AGH) and agrimol C (AGC) all displayed similar cytotoxicity as AGB. These results show that: (i) the length of acyl group of the middle subunit should not exceed five carbons; (ii) A dimeric acylphloroglucinol is essential for the anticancer activities and the third subunit of trimeric acylphloroglucinol is likely to have no contribution to the cytotoxicity; (iii) The tetrameric acylphloroglucinol would not possess any activities.

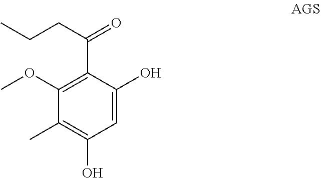 [SMILES: CCCC(=O)c1c(O)cc(O)c(C)c1OC]

TABLE 5
The IC50 values of AGB and its derivatives
determined by MTT method (48 h, μM)

| | AGS | H460 | HeLa | MDA-MB-231 |
|---|---|---|---|---|
| Compounds | cell line | cell line | cell line | cell line |
| AGS | >50 | >50 | >50 | >50 |
| AGB | 2.65 | 1.85 | 0.81 | 2.16 |
| AGC | 8.01 | 1.76 | 1.52 | 2.44 |
| AGH | 5.43 | 2.66 | 1.55 | 2.84 |
| AGF | >50 | >50 | 30.44 | >50 |
| AGL | >50 | >50 | 35.14 | >50 |

Table 6 shows the test results (IC50 values) of some additional AGB structures determined by MTT method (48 h, M).

TABLE 6
The IC50 values of AGB and its derivatives determined by MTT method
(48h, μM)

| Structure | H460 | HeLa | A549 |
|---|---|---|---|
| 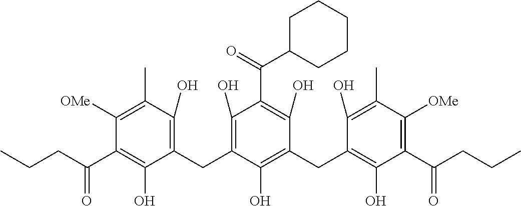 [SMILES: CCCC(=O)c1c(O)c(Cc2c(O)c(Cc3c(O)c(C)c(OC)c(C(=O)CCC)c3O)c(O)c(C(=O)C3CCCCC3)c2O)c(O)c(C)c1OC] | 2.3 | 1.44 | |
| 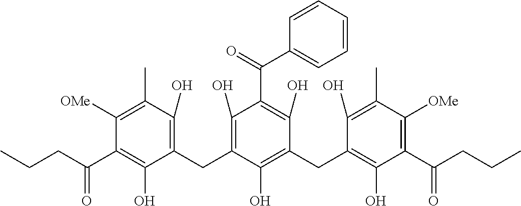 [SMILES: CCCC(=O)c1c(O)c(Cc2c(O)c(Cc3c(O)c(C)c(OC)c(C(=O)CCC)c3O)c(O)c(C(=O)c3ccccc3)c2O)c(O)c(C)c1OC] | 2.75 | 1.23 | 19.3 |
| 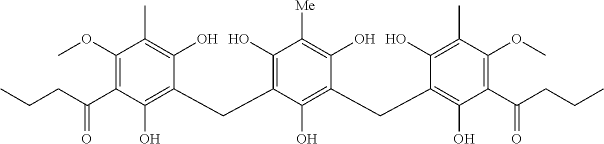 [SMILES: CCCC(=O)c1c(O)c(Cc2c(O)c(C)c(O)c(Cc3c(O)c(C)c(OC)c(C(=O)CCC)c3O)c2O)c(O)c(C)c1OC] | 3.78 | | |
| 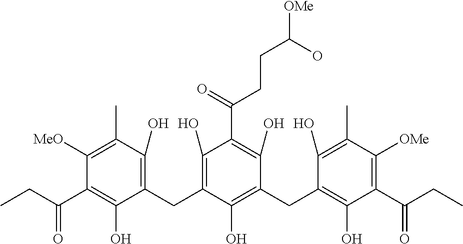 [SMILES: CCC(=O)c1c(O)c(Cc2c(O)c(Cc3c(O)c(C)c(OC)c(C(=O)CC)c3O)c(O)c(C(=O)CCC(O)OC)c2O)c(O)c(C)c1OC] | 4.67 | 3.56 | 41.9 |
| 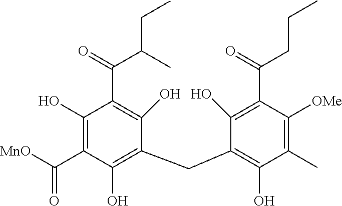 | 0.97 | 1 | 5.52 |
| 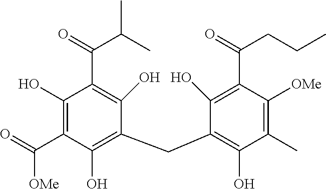 [SMILES: CCCC(=O)c1c(O)c(Cc2c(O)c(C(=O)OC)c(O)c(C(=O)C(C)C)c2O)c(O)c(C)c1OC] | 1.14 | | |
| 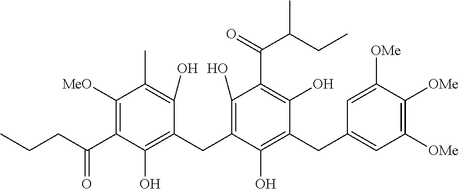 [SMILES: CCCC(=O)c1c(O)c(Cc2c(O)c(Cc3cc(OC)c(OC)c(OC)c3)c(O)c(C(=O)C(C)CC)c2O)c(O)c(C)c1OC] | 2.3 | 2.06 | 8.53 |
| 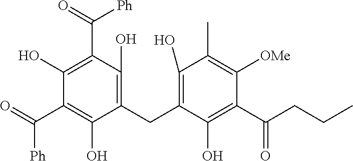 [SMILES: CCCC(=O)c1c(O)c(Cc2c(O)c(C(=O)c3ccccc3)c(O)c(C(=O)c3ccccc3)c2O)c(O)c(C)c1OC] | 3.13 | 3.24 | 28.4 |
| 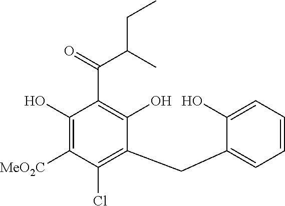 [SMILES: CCC(C)C(=O)c1c(O)c(Cc2ccccc2O)c(Cl)c(C(=O)OC)c1O] | 2.05 | | |
| 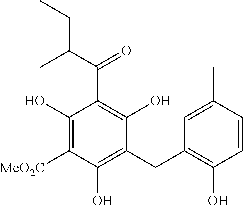 [SMILES: CCC(C)C(=O)c1c(O)c(Cc2cc(C)ccc2O)c(O)c(C(=O)OC)c1O] | 2.99 | 3.96 | 20.5 |

### The ProTargetMiner Analysis

The results above show that AGB has the capacity to induce apoptosis and the underlying mechanism was further investigated by the ProTargetMiner analysis. General differential expression proteomic analysis for investigating protein regulations is not a parameter specific enough for the elucidation of the mechanism of action because of some generic proteins stimulated by all toxic compounds. ProTargetMiner is a tool for studying the mechanism of action in cancer cells. It is a signature library of proteome responses of human cancer cell to anticancer agents. By comparing a given compound against all the other drugs in the library, the specific regulation proteins in response to the given compound can be screened by an orthogonal partial least square discriminant analysis (OPLS-DA). OPLS-DA analysis filters those non-specific regulation so that the specific ones could be obtained to map the network.

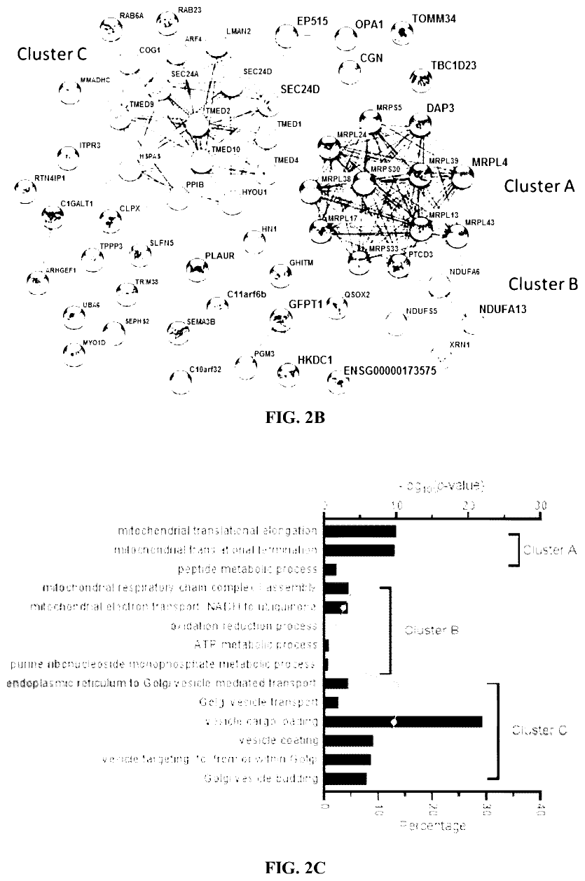

The differential expression of proteins between vehicle group and AGB-treatment group were measured by TMT-labeled quantitative proteomic approach. As a result, the most specifically up-regulated and down-regulated proteins induced by AGB were given at the outlier (FIG. 2A). They mainly belong to vesicle transporter proteins, mitochondrial ribosome proteins (MRPs), and NADH dehydrogenases, respectively. A protein named OPA1 was shown as the most down-regulated protein.

In order to have a more integrated view of the changes in protein complexes involved, a protein-protein interaction network was mapped using STRING to visualize the top-100 most specifically up- or down-regulated proteins affected by AGB. Using an unbiased k-means clustering method, six main clusters of interactions was obtained (Clusters A, B, and C), containing 4, 12, and 14 proteins, respectively. As shown in FIG. 2B, the proportion of proteins in each cluster affected by AGB was grouped and labeled. GO analysis was further performed for these six clusters separately. Proteins in cluster A belong to mitochondrial ribosomal proteins, which are involved in mitochondrial protein translation. Cluster B contains mostly vesicle transport proteins that regulate ER to Golgi trafficking. Cluster C represents proteins for biological function of the mitochondrial respiratory chain complex I assembly (FIG. 2C). The ProTargetMiner analysis results assessed by STRING network analysis approaches revealed that the effects on mitochondria function and ER to Golgi trafficking may play a role for the anticancer activity of AGB.

### Agrimol B Inhibits ER to Golgi Trafficking and Induced ER Stress

Based on the ProTargetMiner analysis results, the mitochondrial function and ER stress were studied. First, the results described in ProTargetMiner analysis show that the inhibition of ER-Golgi trafficking is regulated by AGB. In cells, proteins were initially synthesized in ER and transport to Golgi apparatus for processing. When a drug blocked ER to Golgi trafficking, the Golgi apparatus would suffer from a dramatic redistribution and disruption. This blocking causes accumulation of unmatured proteins in the ER, thereby disrupting the structure of Golgi apparatus and inducing ER stress. The Golgi disrupting agent, brefeldin A (BFA) was used as a positive control drug. BFA is a lactone fungal macrolide antibiotic that specifically inhibit the protein transport by targeting at Golgi apparatus. The effects of BFA and AGB on the integrity of the Golgi complex were assessed by immunostaining using an anti-GM130 antibody, one of Golgi markers. The results show that incubation of the NCI-H460 cells with BFA led to redistribution of GM130, indicating Golgi disruption (data not shown). Similar results were obtained when the cells were exposed to AGB treatment. Thus, the effects of BFA and AGB on Golgi integrity and function were demonstrated to be in a similar mechanism and thereby led to Golgi disruption and ER stress in cancer cells.

The disruption of Golgi structure leads to impaired glycosylation processes, resulting in misfolded proteins which would accumulate to induce ER stress. The relationship between AGB and ER stress in cancer cells was examined. The morphology of ER was disrupted and the content was lessened using ER tracker staining (images not shown). In order to verify that NCI-H460 cells developed ER stress response when treated with AGB, the expression of two ER stress markers, Glucose-regulated protein (GRP) 78 (GRP78) and C/-EBP homologous protein (CHOP), was examined. GAPDH was used as a loading control. The Western Blot images show that GRP78 and CHOP were significantly increased in a time-dependent manner in NCI-H460 cells treated with AGB (data not shown). Generally, there are three sensors to initiate ER stress responses, including IRE1, PERK and ATF6. Upon the accumulation of unfolded proteins, BiP preferentially binds to the unfolded proteins, which results in the release of PERK, IRE1, and ATF6. Once released from BiP, PERK becomes activated and dimerized. Activated PERK phosphorylates eIF2α to suppress the overall transcription of mRNAs. The phosphorylation status of eIF2α in AGB-treated cells was examined, and the results demontrate that eIF2α was phosphorylated in NCI-H460 cell lines.

### AGB Induces Mitochondrial Fission

Another cluster highlighted in the ProTargetMiner analysis was related to mitochondrial translation and respiration function. Time-lapse microscopy was used to monitor the changes of mitochondrial morphology during a 30 hour, 4 μM AGB treatment using U2OS cells transfected with mitochondrial-RFP reporter gene (data not shown). AGB indeed triggers dynamic changes of mitochondria. As shown in the movie recorded using time-lapse microscopy (data not shown), about 5 hours after the start of the imaging, mitochondria changed to a thread-like, very elongated morphology. Then the threads slowly broke up. At about 10 h, the mitochondria became small dots and these dots got brighter. At the end of the movie (about 30 h treatment), some cells underwent mitochondrial outer membrane permeabilization (the abrupt loss of fluorescence in mitochondria) and died. These results show that cells underwent a mitochondrial fission after the treatment of AGB.

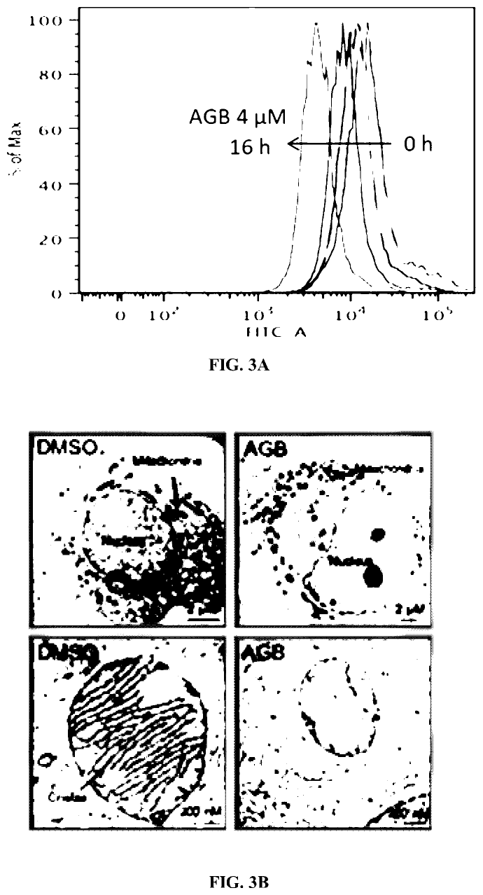

Mitochondria are highly dynamic organelles that frequently fuse and fragment, with the ability to change size, shape, and position. The dynamic nature of mitochondria is important for mitochondrial inheritance and for the maintenance of mitochondrial functions. When the balance between fission and fusion is disrupted, as result of a relative increase or decrease in either process, the overall mitochondrial morphology is changed markedly. Using confocal microscopy analysis by MitoTracker Green staining, the mitochondria of NCI-H460 cells in the untreated control groups exhibited normal elongated-tubular structures. In contrast, after exposure to 4 μM AGB for various time periods (0, 2, 5, 8, 10, and 16 h, respectively), the morphology of mitochondria gradually changed into significant punctuated structures (staining data not shown), and then the fluorescence signal disappeared after 16 h (FIG. 3A).

Several proteins that control mitochondrial fusion and fission have been identified, including mitofusins and OPA1 for fusion, and Mff and DRP1 for fission. The effects of AGB on the expression of mitochondrial fusion proteins (Mfn1 and Mfn2) and fission proteins (DRP1 and Mff) were studied. Western Blot analysis showed that AGB slightly decreased the expression of Mfn1 and Mfn2 in a dose-dependent manner, demonstrating that the fusion process was affected (data not shown). The Western Blot images also showed a significant decrease in phosphor-DRP1 (Ser637) while AGB did not alter the expression of DRP1 and Mff (data not shown). DRP1 phosphorylation at residue S637 by protein kinase A inhibits mitochondrial fission whereas dephosphorylation at S637 increases mitochondrial recruitment of DRP1 and promotes mitochondrial fission. The localization of DRP1 was examined by the immunofluorescence staining. The immunostaining of Drp1 in the mito-RFP-transfected cells showed that AGB significantly promoted the translocation of DRP1 from cytoplasm into mitochondria (data not shown), demonstrating that AGB induced a DRP1-mediated mitochondrial fission.

OPA1 is another protein localized at the inner mitochondrial membrane and is involved in different function like regulation of mitochondrial morphology dynamics, maintenance of respiratory chain, and control of ATP synthesis. The decrease of L-OPA1 causes inhibition of mitochondrial fusion, whereas the accumulation of S-OPA1 facilitates mitochondrial fission. From the results of ProTargetMiner, OPA1 was found to be the most specifically down-regulated proteins by OPLS-DA analysis. These results were verified using Western Blot, demonstrating that AGB treatment promoted OPA1 cleavage from its long form (L-OPA1) into the short form (S-OPA1) both in a dose and time dependent manner (data not shown). OPA1 plays a role in maintaining the mitochondrial cristae structure. OPA1 fragmentation leads to a disorganization of the structure of the mitochondrial inner membrane. The ultrastructure of mitochondria was observed under transmission electron microscopy (TEM) with the treatment of AGB or vehicle for 16 h. Classical long tubular cylinders were observed in the control group, while circular vesicles of reduced diameter predominated in AGB treatment group, confirming a mitochondrial fission observed above. Moreover, the cristae structure of mitochondria from treatment group demonstrated completely unstructured and adopted unusual shapes composed of vesicle-like structures with abnormally increased space between the membranes (FIG. 3B). Collectively, these observations demonstrate that AGB induced mitochondrial fission.

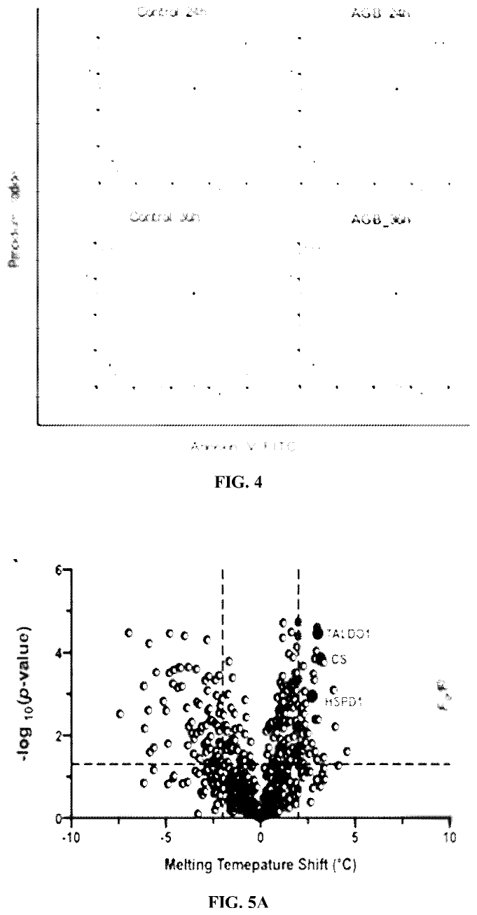

The mitochondrial fission and mitochondrial membrane potential depolarization are involved in the induction of cell apoptosis and also play a central role to the apoptotic pathway. Therefore, several apoptosis assays were performed. First, Hoechst staining showed chromatin condensation and fragmentation, which is one of the characteristics of apoptosis (images not shown). Flow cytometry analysis by annexin V and PI staining also revealed the increase of the early and late apoptotic cell populations after AGB treatment (FIG. 4). Induction of apoptosis by AGB in NCI-H460 cells was further supported through monitoring PARP, caspase-9, and caspase-3 cleavage as well as through changes in the levels of a panel of proteins commonly involved in apoptotic signaling via Western blotting experiments (data not shown). A release of cytochrome c and apoptosis inducing factor AIF from mitochondria to cytosol were also observed (data not shown), demonstrating that mitochondria was involved in apoptosis pathway.

### Target Identification of AGB Using Label-Free Approaches

Thermal proteome profiling (TPP) and proteome integral solubility alteration (PISA) methods were applied to assess the molecular targets of AGB in living cells without a structural modification and in an unbiased way. TPP and PISA are two strategies using mass spectrometry (MS)-based proteomics, which allow observation of changes in thermal stability of the entire cellular proteome and identification of molecular targets once a stabilization is shown. After 2 h-treatment of AGB, both groups of cells were heated to different temperature from 37° C. to 67° C. to induce thermal denaturation. Then the cells were lysed and insoluble proteins were removed by ultracentrifugation. The remaining soluble proteins were quantified by LC-MS/MS. In total, 42 and 74 putative AGB binding proteins were identified from NCI-H460 cells by TPP (ΔTₘ>2° C., p<0.05) and PISA (fold change >1.5, p<0.05), respectively. Three (3) mutual proteins (HSPD1, CS, and TALDO1) were identified using both methods.

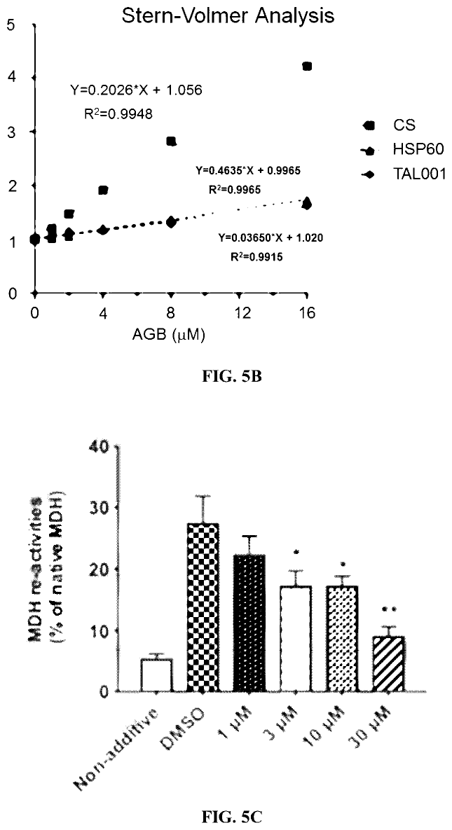

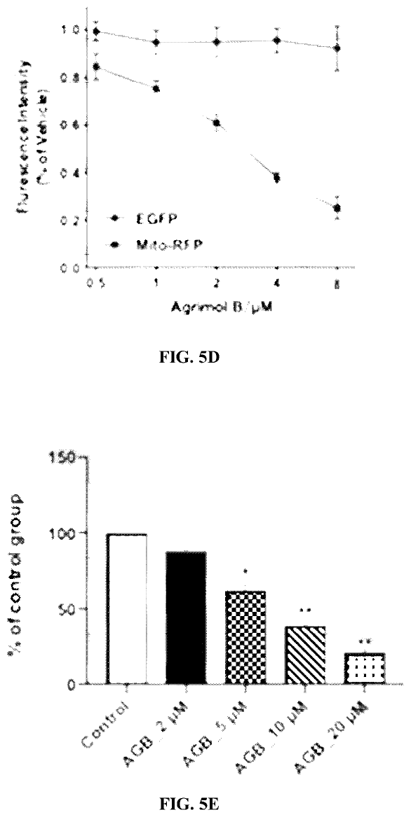

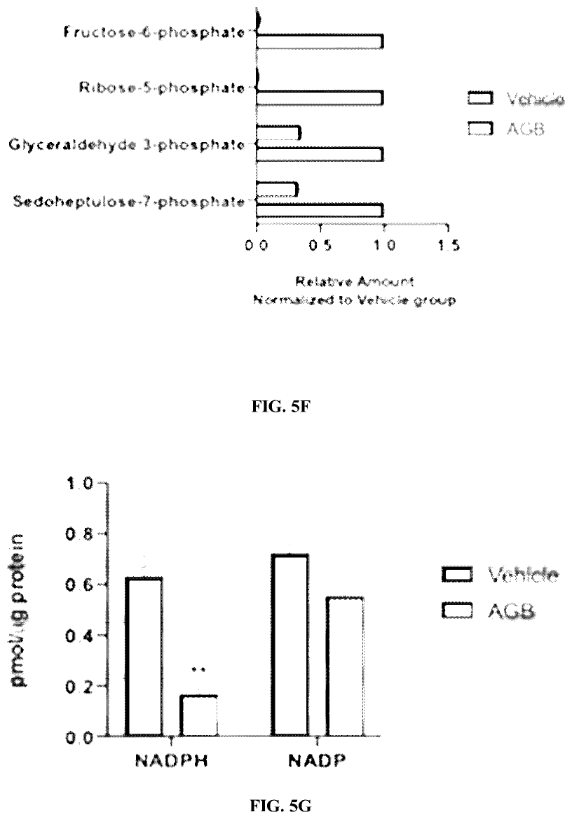

From the GO enrichment analysis for 200 specifically regulated proteins by AGB treatment, the proteins that were mostly enriched in six GO terms with the best z-scores and most significant p-values are involved in cellular metabolic process, mitochondrial translational elongation, NADH dehydrogenase activity, mitochondrial respiratory chain complex assembly, carbohydrate derivative biosynthetic process, endoplasmic reticulum to Golgi vesicle-mediated transport, indicative of the general functional perturbation in NCI-H460 under AGB treatment. Amongst these identified mutual proteins, TALDO1 is a key enzyme of the non-oxidative pentose phosphate pathway (PPP), which is responsible for regulation of cell metabolism. CS is the enzyme regulating the first step of the citric acid cycle in order to maintain ATP supplement in cell. HSP60 together with HSP10 are mainly involved in the folding process of the imported proteins. These three proteins may be the key targets of AGB (FIG. 5A). CS, TALDO1 and HSP60 were further studied to determine the relevant biological pathways affected by AGB.

Immunoblot experiments were carried out for validating of the three identified potential targets. For HSP60, AGB treatment resulted in a significant increase in the protein melting temperature (Tₘ) by 3.2° C. compared to DMSO control, as revealed in the plots of CETSA melt curve obtained from quantitation of the western blot signals (data not shown). In the isothermal dose-response fingerprint (ITDRF_{CETSA}) experiment, treatment of cells with increasing concentrations of AGB led to dose dependent recovery of soluble HSP60 in cells subjected to heating at protein denaturation temperature at 60° C. For CS and TALDO1, similar protein stabilization effects upon treatment of cells with AGB were observed in both CETSA and ITDRF_{CETSA} experiments (data not shown). These results are consistent with the results from TPP, demonstrating the cellular engagements of AGB to CS, TALDO1 and HSP60 in NCI-H460 cells.

To further examine whether AGB interacts directly with the above three identified proteins, the intrinsic tryptophan fluorescence properties of these proteins were examined in the presence of AGB at different concentrations. Upon binding with AGB, proteins experience a change of the tryptophan environment, which can be visualized by fluorescence spectroscopy. As shown in FIG. 5B the tryptophan fluorescence of three proteins were all quenched by AGB in a concentration-dependent manner. Base on the change in fluorescence induced by AGB, Stern-Volmer analysis determined that the K_{d} values of AGB binding to HSP60, CS and TALDO1 are 27.02 μM, 4.9 μM, and 22.73 μM, respectively. According to the IC₅₀ value of AGB (2.3 μM for 48 h) and the effective concentration for biological experiments (4 μM in usual), the K_{d} constant of CS is close to but slightly higher for HSP60 and TALDO1 (FIG. 5B). These results revealed that the contribution of these identified target protein may be different.

The function of HSP60 is mainly for its chaperone activity. HSP60 cooperates with co-chaperonin HSP10 to perform the misfolded proteins refolding and reactivate. Mitochondrial malate dehydrogenase (MDH), one of HSP60 substrates, was examined for the inhibition of chaperone function by AGB. By exclusion of the direct impacts of AGB on MDH function, a pre-incubation of Hsp60 with different concentration of AGB, the refolding activities showed a concentration-dependent inhibition (FIG. 5C). In addition, mitochondrial targeted red fluorescence protein (Mito-RFP) is a Hsp60 client, only exhibiting fluorescence when it is properly folded in mitochondria. The fluorescent imaging data shows that in cells transfected with Mito-RFP, treatment with AGB resulted in a significant reduction of mitochondrial fluorescence compared to vehicle treatment (FIG. 5D). In contrast, cells transfected with an EGFP-expressing construct with no organelle-targeting property did not show changes in fluorescence under AGB treatment.

CS is localized within the mitochondrial matrix and is the key enzyme in the first step of the tricarboxylic acid (TCA) cycle that catalyzes the reaction between acetyl coenzyme A (acetyl CoA) and oxaloacetic acid to form citric acid. After the treatment with different concentrations of AGB, a significant decrease in the enzyme activity was observed at 5 μM of AGB and the inhibition effects were displayed in a dose-dependent manner (FIG. 5E). CS is responsible for the production of citrate in the TCA cycle that mediates mitochondrial aerobic respiration to affect the production of ATP production. Targeting at CS may be feasible to restore the abnormal expression of TCA cycle. The results show that AGB may play a role as an inhibitor of CS for treating the lung cancer.

TALDO1 is a key enzyme of the nonoxidative PPP providing ribose-5-phosphate for nucleic acid synthesis and NADPH (the reduced form of Nicotinamide adenine dinucleotide phosphate) for lipid biosynthesis. Targeted metabolomics analysis was performed for measuring of the intermediate metabolites involved in the PPP pathway. As a result, four important intermediate metabolites involved in TALDO1-catalyzed pathway, including fructose-6-phosphate, ribose-4-phosphate, glyceraldehyde-3-phosphate, and sedoheptulose-7-phosphate, significantly decreased after the treatment of AGB (FIG. 5F). Moreover, the intracellular content of NADPH was measured to show a significant decrease, showing that the activity of TALDO1 was inhibited by AGB (FIG. 5G). PPP activation has been widely found in different types of cancer because of the uncontrolled proliferation of cancer cells. Enhanced PPP would facilitate nucleic acid synthesis and hence produce NAPDH for fatty acid and cholesterol biosynthesis. Moreover, enhanced production of NADPH is crucial to increase the intracellular redox power of cancer cells to escape from oxidative stress. Thus, AGB not only reduced the intermediate metabolites of PPP for the material supply of nucleic acid synthesis but also decreased the generation of NADPH through targeting with TALDO1.

The above results showed that three proteins (HSP60, CS, and TALDO1) meet all requirements of thermal stabilization. The direct effects of AGB on function of these three proteins were also further validated.

In summary, the total synthesis of AGB and its derivatives was improved and the underlying mechanisms of AGB along with its molecular targets were also elucidated, demonstrating that AGB is a promising anticancer agent.

### REFERENCES

Akbar, et al., Journal of bioenergetics and biomembranes 48 (2016) 241-247.
Alavi and Fuhrmann, Molecular neurodegeneration 8 (2013) 1-12.
Alp, et al., Biochemical Journal 154 (1976) 689-700.
Al-Shamsi, et al., European journal of pediatrics 174 (2015) 661-668.
Bajaj, et al., Journal of Cell Biology 219 (2020).
Bradbury, et al., Clinical lung cancer 18 (2017) 259-273. e8.
Cappello, et al., Cancer biology & therapy 7 (2008) 801-809.
Cereghetti, et al., P.N.A.S. 105 (2008) 15803-15808.
Chan, Cell 125 (2006) 1241-1252.
Chang and Blackstone, Journal of Biological Chemistry 282 (2007) 21583-21587.
Chen, et al., Nature metabolism 1 (2019) 404-415.
Chen, et al., PloS one 9 (2014) e115708.
Choi, et al., J Korean Oriental Med 30 (2009) 129-41.
Cnop, et al., Molecular metabolism 6 (2017) 1024-1039.
Frank, et al., BBA-Molecular Cell Research 1823 (2012) 2297-2310.
Frezza, et al., Cell 126 (2006) 177-189.
Gaetani, et al., Journal of proteome research 18 (2019) 4027-4037.
Gao, Journal of cellular and molecular medicine 12 (2008) 67-96.
Gordaliza, Clinical and Translational Oncology 9 (2007) 767-776.
Gordaliza, Clinical and Translational Oncology 9 (2007) 767-776.
Griparic, et al., Journal of Cell Biology 178 (2007) 757-764.
Hartl, et al., Annual review of biophysics and biomolecular structure 21 (1992) 293-322.
Hnit, et al., Biomedicine & Pharmacotherapy 141 (2021) 111795.
Hong, et al., Frontiers in pharmacology 9 (2018) 43.
Jiang, et al., Protein & cell 5 (2014) 592-602.
Klausner, et al., The Journal of cell biology 116 (1992) 1071-1080.
Kopp, et al., Nature structural & molecular biology 26 (2019) 1053-1062.
Koshiura, et al., The Japanese Journal of Pharmacology 38 (1985) 9-16.
Larochelle, et al., Molecular and cellular biology 26 (2006) 6690-6701.
Lee and Hendershot, Cancer biology & therapy 5 (2006) 721-722.
Lee, et al., Molecular biology of the cell 15 (2004) 5001-5011.
Li, et al., Acta Chinica Sinica 36 (1978) 43-48.
Lin, et al., Scientific reports 2 (2012) 1-12.
Ly, et al., Apoptosis 8 (2003) 115-128.
Matsuno-Yagi and Hatefi, Biochemistry 28 (1989) 4367-4374.
Patra and Hay, Trends in biochemical sciences 39 (2014) 347-354.
Pei, et al., Acta Pharmaceutica Sinica 24 (1989) 431-437.
Pharmacopoeia of the People's Republic of China, Chemical Industry Press (2020).
Reers, et al., Methods in enzymology 260 (1995) 406-417.
Rozpedek, et al., Current molecular medicine 16 (2016) 533-544.
Saei, et al., Nat Commun 10 (2019) 5715.
Savitski, et al., Science 346 (2014).
Schlichtholz, et al., Pancreas 30 (2005) 99-104.
Senft and Ze'ev, Current opinion in cell biology 39 (2016) 43-52.
Sheridan and Martin, Mitochondrion 10 (2010) 640-648.
Siegel, et al., CA: a cancer journal for clinicians 71 (2021) 7-33.
Song, et al., Signal transduction and targeted therapy 5 (2020) 1-19.
Srinivasan, et al., BBA-Bioenergetics 1858 (2017) 602-614.
Szklarczyk, et al., Nucleic acids research 43 (2015) D447-D452.
Verschueren, et al., Nature 568 (2019) 571-575.
Wiegand and Remington, Annu Rev Biophys Biophys Chem 15 (1986) 97-117.
Xie, et al., Signal transduction and targeted therapy 5 (2020) 1-30.
Xu, et al., Cell 182 (2020) 245-261. e17.
Yang and Luo, Biomolecules 5 (2015) 2538-2553.
Yoshida, et al., Experimental cell research 192 (1991) 389-395.
Youle and Karbowski, Nature reviews Molecular cell biology 6 (2005) 657-663.
Youle and Van Der Bliek, Science 337 (2012) 1062-1065.
Zhu, et al., Molecules 22 (2017).

1.-2. (canceled)

3. A compound having the structure of 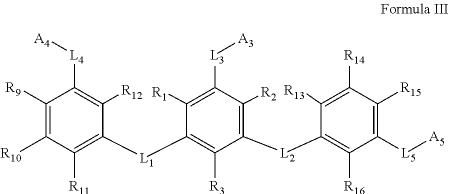
wherein:
(a) L₁ and L₂ are independently a single bond or a substituted or unsubstituted alkylene;
(b) R₁—R₃ are hydroxyl;
(c) -L₃-A₃ is a substituted or unsubstituted alkyl, or 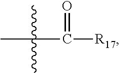
R₁₇ is a substituted or unsubstituted alkyl, a substituted or unsubstituted aryl, or 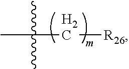
m is an integer from 0 to 5 and R₂₆ is a carboxylic acid, an ester, an amino, or an amide;
(d) L₄ and L₅ are independently a single bond, a substituted or unsubstituted alkylene, or a carbonyl;
(e) A₄ and A₅ are independently a hydrogen, a substituted or unsubstituted alkyl, a substituted or unsubstituted aryl, a carbonyl, or an alkoxy;
(f) R₉—R₁₆ are independently a hydrogen, a hydroxyl, a substituted or unsubstituted alkyl, a carbonyl, or an alkoxy; and
(g) the substituents, when present, are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl,
with the proviso that:
(i) when -L₃-A₃, -L₄-A₄, and -L₅-A₅ are 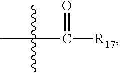
and R₁₇ is an unsubstituted C₁-C₄ alkyl, then -L₃-A₃ is not —C(O)-(n-propyl) and not —C(O)-(isobutyl); and
(ii) when -L₃-A₃, -L₅-A₅, and R₁₀ are 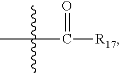
and R₁₇ is an unsubstituted C₁-C₃ alkyl, then -L₃-A₃ is not —C(O)-(n-propyl) and not —C(O)-(isopropyl).

4. (canceled)

5. The compound of claim 3, wherein:
R₉ and R₁₁—R₁₆ are independently a hydrogen, a hydroxyl, an unsubstituted alkyl, —OR₄, —SR₅, or —NR₆R₇, R₄—R₇ are independently a hydrogen or a substituted or unsubstituted alkyl; and/or
R₁₀ is an unsubstituted alkyl or a carbonyl.

6. (canceled)

7. The compound of claim 3, wherein R₉ and R₁₁—R₁₆ are independently a hydrogen, a hydroxyl, an unsubstituted C₁-C₆ alkyl, or —OR₄, R₄ is an unsubstituted C₁-C₆ alkyl; and/or
R₁₀ is an unsubstituted C₁-C₆ alkyl or 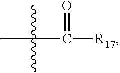
R₁₇ is an unsubstituted C₁-C₆ alkyl.

8.-9. (canceled)

10. The compound of claim 3, wherein R₁₇ is an unsubstituted C₁-C₆ alkyl, a C₆ cycloalkyl, an unsubstituted phenyl, or 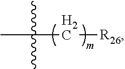
m is an integer from 0 to 5 and R₂₆ is an ester.

11.-12. (canceled)

13. The compound of claim 3, wherein -L₄-A₄ is an unsubstituted C₁-C₆ alkyl or 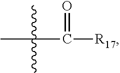
R₁₇ is an unsubstituted C₁-C₆ alkyl; and -L₅-A₅ is 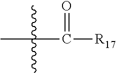
or —OR₄, R₄ is an unsubstituted C₁-C₆ alkyl, R₁₇ is an unsubstituted C₁-C₆ alkyl.

14. A compound having the structure of 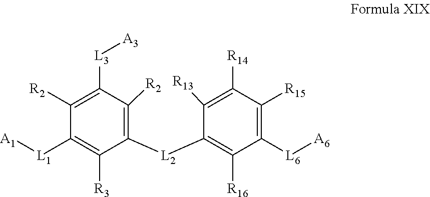
wherein:
(a) L₂ is a single bond or an unsubstituted alkylene;
(b) L₁ is —C(O)—, and A₁ is a substituted or unsubstituted alkyl, a substituted or unsubstituted aryl, or an alkoxy;
(c) R₁—R₃ are a hydroxyl;
(d) R₁—R₁₆ are independently a hydrogen, a hydroxyl, a substituted or unsubstituted alkyl, a carbonyl, —OR₄, —SR₅, or —NR₆R₇, R₄—R₇ are independently a hydrogen or a substituted or unsubstituted alkyl;
(e) -L₃-A₃ is 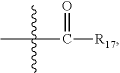
R₁₇ is a substituted or unsubstituted alkyl, a substituted or unsubstituted aryl;
(f) -L₅-A₅ is a hydrogen, a substituted or unsubstituted alkyl, or 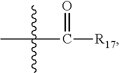
R₁₇ is a substituted or unsubstituted alkyl; and
(g) the substituents, when present, are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl,
with the proviso that:
(i) when -L₁-A₁ and -L₃-A₃ are the same 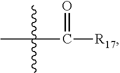
then R₁₇ is not methyl.

15.-17. (canceled)

18. The compound of claim 14, wherein -L₃-A₃ is 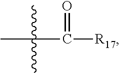
R₁₇ is an unsubstituted phenyl or an unsubstituted C₁-C₆ alkyl.

19. The compound of claim 14, wherein -L₁-A₁ is 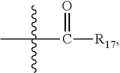
R₁₇ is an unsubstituted phenyl or —OR₄, R₄ is an unsubstituted C₁-C₆ alkyl.

20. (canceled)

21. The compound of claim 3, wherein the compound is

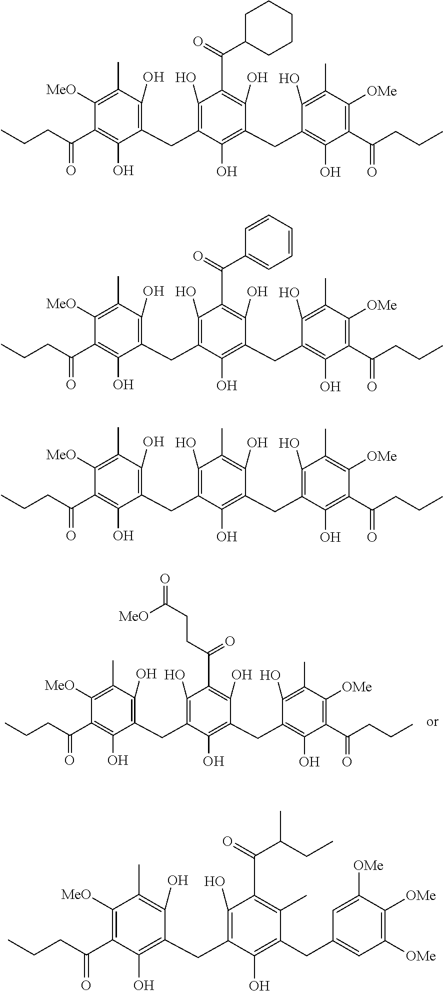

22. A pharmaceutical formulation comprising
one or more compounds of claim 3; and
a pharmaceutically acceptable carrier and/or excipient.

23. The pharmaceutical formulation of claim 22, wherein the one or more compounds are in an effective amount to treat a cancer, reduce a cancer, or treat or ameliorate one or more symptoms associated with a cancer in a subject.

24. The pharmaceutical formulation of claim 23, wherein the effective amount of the one more compounds is effective to:
(a) induce mitochondria dysfunction and/or to inhibit ER to Golgi trafficking in cancer cells in the subject;
(b) induce apoptosis of cancer cells in the subject; and/or
(c) inhibit the growth of a tumor by at least 40%, at least 50%, or at least 55% in the subject compared to the same tumor in a control subject.

25.-29. (canceled)

30. A method for synthesizing a compound of Formula I, 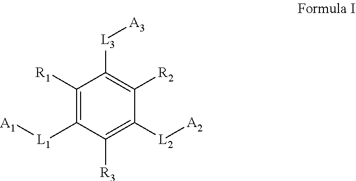
the method comprising:
(i) heating a reaction mixture at a suitable temperature for a period of time sufficient to form a product comprising the compound, wherein the reaction mixture comprises a solvent, a first reactant having the structure of Formula V, 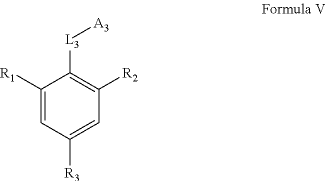
a second reactant having the structure of Formula VI, and 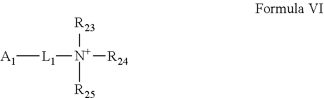
a third reactant having the structure of Formula VII, 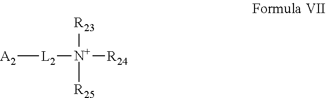
wherein
(a) L₁ and L₂ are independently a bond (single, double, or triple), a substituted or unsubstituted alkyl, a substituted or unsubstituted alkenyl, a carbonyl, or an alkoxy;
(b) L₃ is a bond (single, double, or triple), a substituted or unsubstituted alkyl, or a carbonyl;
(c) A₁ and A₂ are independently a hydrogen, a substituted or unsubstituted alkyl, a substituted or unsubstituted alkenyl, a substituted or unsubstituted aryl, a carbonyl, or an alkoxy, and at least one of A₁ and A₂ is a substituted or unsubstituted aryl;
(d) A₃ is a hydrogen, a substituted or unsubstituted alkyl, a substituted or unsubstituted aryl, a carbonyl, or an alkoxy;
(e) R₁—R₃ are independently a hydrogen, a hydroxyl, a substituted or unsubstituted alkyl, a carbonyl, an alkoxy, a thiol, or an amino;
(f) R₂₃—R₂₅ are independently a hydrogen or a substituted or unsubstituted alkyl; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl, and
wherein the second reactant is the same as or different from the third reactant.

31.-40. (canceled)

41. The method of claim 30, wherein the compound has an overall yield of at least 7%, at least 10%, at least 15%, at least 20%, at least 25%, or at least 30%.

42. (canceled)

43. A method for treating a cancer, reducing a cancer, or treating or ameliorating one or more symptoms associated with a cancer in a subject comprising:
(i) administering to the subject the pharmaceutical formulation of claim 22, wherein step (i) occurs one or more times.

44. The method of claim 43, wherein the method comprises only a single administration of the pharmaceutical formulation, wherein the pharmaceutical formulation comprises an effective amount of the compounds to inhibit the growth of a tumor by at least 40%, at least 50%, or at least 55% in the subject compared to the same tumor in a control subject, or
wherein the method comprises more than one step of administering to the subject the pharmaceutical formulation, wherein following all of the administration steps an effective amount of the compounds to inhibit the growth of a tumor by at least 40%, at least 50%, or at least 55% in the subject compared to the same tumor in a control subject is administered to the subject.

45. The method of claim 44, wherein the effective amount of the compounds is in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 3 mg/kg.

46.-47. (canceled)

48. The method of claim 43, wherein the cancer is non-small-cell lung cancer.

49.-56. (canceled)

57. The compound of claim 14, wherein the compound is

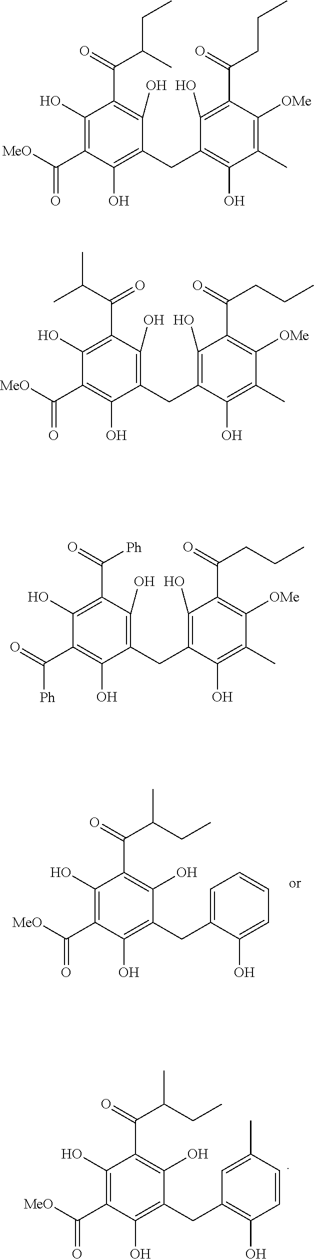 [SMILES: CCCC(=O)c1c(O)c(Cc2c(O)c(C(=O)OC)c(O)c(C(=O)C(C)CC)c2O)c(O)c(C)c1OC ; CCCC(=O)c1c(O)c(Cc2c(O)c(C(=O)OC)c(O)c(C(=O)C(C)C)c2O)c(O)c(C)c1OC ; CCCC(=O)c1c(O)c(Cc2c(O)c(C(=O)c3ccccc3)c(O)c(C(=O)c3ccccc3)c2O)c(O)c(C)c1OC ; CCC(C)C(=O)c1c(O)c(Cc2ccccc2O)c(O)c(C(=O)OC)c1O ; CCC(C)C(=O)c1c(O)c(Cc2cc(C)ccc2O)c(O)c(C(=O)OC)c1O]

58. A pharmaceutical formulation comprising
one or more compounds of claim 14; and
a pharmaceutically acceptable carrier and/or excipient.

59. A method for treating a cancer, reducing a cancer, or treating or ameliorating one or more symptoms associated with a cancer in a subject comprising:
(i) administering to the subject the pharmaceutical formulation of claim 58, wherein step (i) occurs one or more times.

60. The method of claim 59, wherein the cancer is non-small-cell lung cancer.
